(19) United States
(12) Patent Application Publication
(10) Pub. No.: US 2022/0251658 A1
(43) Pub. Date: Aug. 11, 2022

(54) METHOD FOR ESTABLISHING AN INDIVIDUAL PHYSICAL ACTIVITY PROGRAM FOR A SUBJECT FOR REDUCING AN INDIVIDUAL RISK OF THE SUBJECT FOR DEVELOPING A CARDIOVASCULAR DISEASE

(71) Applicant: Westfaelische Wilhelms-Universitaet Muenster, Munich (DE)

(72) Inventor: Boris Schmitz, Nottuln (DE)

(21) Appl. No.: 17/622,149

(22) PCT Filed: Jun. 26, 2020
(86) PCT No.: PCT/EP2020/067980

§ 371 Date: Dec. 22, 2021

(30) Foreign Application Priority Data
Jun. 26, 2019 (LU) LU101280

(51) Int. Cl. C12Q 1/6883; G16H 50/30; G16H 10/40

(52) U.S. Cl. CPC C12Q 1/6883; C12Q 1/6851; G16H 10/40; G16H 50/30

(57) ABSTRACT

The present invention relates to a method for establishing an individual physical activity program for a subject for reducing an individual risk of the subject for developing a cardiovascular disease, comprising the following steps: (i) determining the concentration of at least one circulating miRNA in at least one fluid sample obtained from the subject, at least before and after the subject has conducted physical activity; wherein the at least one circulating miRNA is selected from certain miRNAs; (ii) comparing the in step (i) determined concentration(s), wherein the result of this comparison is indicative of whether said subject has an individual risk for developing a cardiovascular disease under certain conditions; and (iii) establishing the individual physical activity program for the subject based on the result of step (ii). The present invention further relates to various uses of miRNAs in any of the methods according to the present invention.

### FIELD OF THE INVENTION

The present invention relates to a method for establishing an individual physical activity program for a subject for reducing an individual risk of the subject for developing a cardiovascular disease, comprising the following steps: (i) Determining the concentration of at least one circulating miRNA in at least one fluid sample obtained from the subject at least before and after the subject has conducted physical activity, wherein the at least one circulating miRNA is selected from the group consisting of hsa-miRNA-1, hsa-miRNA-24, hsa-miRNA-96, hsa-miRNA-143, hsa-miRNA-98, hsa-miRNA-125a, hsa-miRNA-132, and any combination, sub-combination, portion or fragment thereof; (ii) comparing the in step (i) determined concentration(s), wherein the result of this comparison is indicative of whether said subject has an individual risk for developing a cardiovascular disease, if the result of this comparison shows an increase or decrease of the concentration of the at least one circulating miRNA after the subject has conducted physical activity compared to the concentration of the at least one circulating miRNA before the subject has conducted physical activity; and (iii) establishing the individual physical activity program for the subject based on the result of step (ii). Further, the present invention also relates to the use of certain miRNAs in any of the methods according to the present invention.

### BACKGROUND OF THE INVENTION

microRNAs (miRNAs) have been identified as pivotal modulators of the systemic response to physical exercise and subsequent training adaptations [1-4]. Since the general knowledge on miRNAs and their specific targets and functions has greatly increased (see [5] for comprehensive review), miRNAs may also hold the potential to serve as functional biomarkers indicating physiological processes involved in the response to specific training regimes [1, 2, 4].

miRNAs are short (˜21-23 nucleotide-long) non-coding RNAs involved in translational repression [6, 7] regulating a wide range of different physiological processes including development and aging as well as disease [8-10]. Moreover, it has been estimated that up to 60% of all human protein-coding genes are conserved miRNA targets [11]. The myocardium and vascular endothelium are an abundant source for miRNAs that are selectively secreted into the blood stream where they can be detected as circulating miRNAs (c-miRNAs) [12]. These c-miRNAs are preserved by association with RNA-binding proteins or small membranous vesicles and commonly involved in inter-cell communication with active regulation of target cell gene expression [13, 14].

c-miRNA production and secretion is responsive to different stimuli induced by physical exercise including shear stress and hypoxia [15-17]. To this respect, it has been suggested that induction of hemodynamic stimuli including transmural pressure and (episodic) shear stress [18-20] may be key mechanisms responsible for the beneficial impact of physical exercise on vascular function [21, 22].

It has also been noted that the vascular endothelium is an important ‘mechano-sensor’ transducing hemodynamic signals, which may result in flow-induced conversion of endothelial cells into an elongated arterial phenotype as well as in functional and structural changes of the overall arterial wall [20, 23]. Of note, vascular maladaptations including endothelial cell stiffening, disturbed endothelial barrier function and reduced nitric oxide (NO) production [24, 25] as well as the development of atherosclerotic lesions and plaque formation is mainly found in regions with disturbed flow, which increases the secretion of pro-inflammatory molecules and most likely alters miRNA expression [26, 27]. By contrast, these deleterious changes are mostly absent from regions with constant laminar flow [26].

While in vitro and ex vivo shear stress experiments have linked an increase in mean shear stress (i.e. constant laminar shear stress) to local anti-atherosclerotic changes, it has been noted that beneficial effects of exercise on vascular function also occur in arteries that are not subjected to a direct increase in shear stress [18, 23].

To this end, selectively released miRNAs preserved by association with small membranous vesicles or RNA-binding proteins may be involved [13, 14]. These distal effects might include, for example, miRNA-dependent regulation of endothelial proliferation as shown for miR-126 [28], vascular smooth muscle plasticity as reported for miR-145/-143 [29] and many more [15].

However, even if the process and molecular mechanisms involved in miRNA expression regulation and secretion, especially in response to physical exercise, is still incompletely understood [30, 31]. It has been suggested that miRNA-releasing cells may possess a sorting mechanism, guiding specific miRNAs to enter exosomes resulting in the concentration of selected miRNAs [32].

To this end, in vitro shear stress experiments have been shown to alter not only the quantity of exosomes, but also the protein and/or (mi)RNA content of exosomes derived from endothelial cells [33, 34].

The inventors of the present invention used different training protocols including high-intensity interval training (HIIT) protocols to characterize conditions, which lead to the expression of c-miRNAs, such as miRNA-1, -24, -143, -98, -125a, -132 and -96.

Compared to endurance training, HIIT is marked by brief bursts of near-maximal to supra-maximal work rates, followed by short periods of rest or active recovery, accompanied by an overall reduction in training duration. Since the optimal HIIT conditions in terms of intensity and work/rest ratio are still under debate [35-37], miRNAs may also be used to indicate most effective HIIT variants. Vice versa, HIIT may be used to identify miRNAs associated with adaptations to physical training. This is also of interest since HIIT is an efficient tool to improve health-related fitness in the general population and for the prevention of lifestyle-induced chronic diseases.

While recent progress in array and sequencing technologies has entered the field of exercise physiology to identify novel exercise-dependent miRNAs, already available data sets might be used for the discovery of new exercise-inducible miRNAs.

The inventors of the present invention identified miRNAs to be inducible by high intensity exercise, which may be involved in vasculoprotective effects of HIIT.

Thus, the inventors of the present invention have developed an effective method for establishing an individual activity program for a subject for reducing an individual risk of the subject for developing a cardiovascular disease for addressing the above mentioned needs.

### SUMMARY OF THE INVENTION

The present invention relates to a method for establishing an individual physical activity program for a subject for reducing an individual risk of the subject for developing a cardiovascular disease, comprising the following steps: (i) Determining the concentration of at least one circulating miRNA in at least one fluid sample obtained from the subject at least before and after the subject has conducted physical activity, wherein the at least one circulating miRNA is selected from the group consisting of hsa-miRNA-1, hsa-miRNA-24, hsa-miRNA-96, hsa-miRNA-143, hsa-miRNA-98, hsa-miRNA-125a, hsa-miRNA-132, and any combination, sub-combination, portion or fragment thereof; (ii) comparing the in step (i) determined concentration(s), wherein the result of this comparison is indicative of whether said subject has an individual risk for developing a cardiovascular disease, if the result of this comparison shows an increase or decrease of the concentration of the at least one circulating miRNA after the subject has conducted physical activity compared to the concentration of the at least one circulating miRNA before the subject has conducted physical activity; and (iii) establishing the individual physical activity program for the subject based on the result of step (ii).

In one embodiment of the method of the present invention, step (i) comprises determining the concentration of 2, 3, 4, 5, 6, or all 7 of the miRNAs selected from the group consisting of hsa-miRNA-1, hsa-miRNA-24, hsa-miRNA-96, hsa-miRNA-143, hsa-miRNA-98, hsa-miRNA-125a, hsa-miRNA-132, and any combination, sub-combination, portion or fragment thereof.

In another embodiment of the method of the present invention, step (i) comprises determining the concentration of any of the miRNAs selected from the group consisting of hsa-miRNA-1, hsa-miRNA-143, hsa-miRNA-24, hsa-miRNA-96, and any combination, sub-combination, portion or fragment thereof.

In a further preferred embodiment of the method of the present invention, step (i) comprises determining the concentration of any of the miRNAs selected from the group consisting of hsa-miRNA-24, hsa-miRNA-96, and any combination, sub-combination, portion or fragment thereof.

In an embodiment of the method of the present invention, the cardiovascular disease is selected from the group consisting of arteriosclerosis; atherosclerosis; ischemia; endothelial dysfunctions; in particular those dysfunctions affecting blood vessel elasticity; hypertension; peripheral vascular disease; thrombosis; coronary heart disease; heart arrhythmia; heart failure; cardiomyopathy; myocardial infarction; cerebral infarction, renal infarction and restenosis. In another embodiment, the cardiovascular disease may also include those diseases involving chronic inflammatory processes of the vessel wall or elevated pulse wave velocity. In a preferred embodiment of the method of the present invention, the cardiovascular disease is selected from the group consisting of arteriosclerosis; atherosclerosis; ischemia; endothelial dysfunctions; coronary heart disease; myocardial infarction; cerebral infarction, renal infarction and restenosis. In a more preferred embodiment of the method of the present invention, the cardiovascular disease is selected from the group consisting of arteriosclerosis; atherosclerosis; ischemia; coronary heart disease; myocardial infarction; cerebral infarction and renal infarction. In the most preferred embodiment of the method of the present invention, the cardiovascular disease is selected from the group consisting of atherosclerosis, coronary heart disease; myocardial infarction and cerebral infarction.

In one embodiment of the method of the present intervention, the sample is obtained from the subject after the subject has experienced a cardiovascular disease in the past.

In a preferred embodiment of the method of the present invention, the concentration of the at least one circulating miRNA is determined with a polymerase chain reaction- (PCR) based screening such as a real-time quantitative PCR (RT-qPCR) or an immunoassay technique such as a Northern Blot analysis. Preferably, the at least one circulating miRNA is determined by use of a monoclonal antibody for the detection of DNA/RNA dimers.

In one embodiment of the method of the present invention, the at least one fluid sample is further obtained from the subject, while the subject conducts physical activity.

In a further embodiment of the method of the present invention, the sample is obtained from the subject before the subject has conducted physical activity. For example, the sample is obtained at least 4 weeks before the subject conducts physical activity.

In one embodiment of the present invention, the sample is obtained from the subject before the subject has conducted physical activity. For example, the sample is obtained at least 24 hours before the subject conducts physical activity.

In a further embodiment of the method of the present invention, the concentration of the at least one circulating miRNA is determined in a regular time schedule, preferably wherein the regular time schedule comprises 2 days to 52 weeks or one week to 10 years.

In a preferred embodiment of the method of the present invention, the at least one fluid sample is a blood sample, a sample of blood components, a salivary sample, a urine sample, a sweat sample or a lymph sample.

In one embodiment of the method of the present invention, establishing the individual physical activity program for the subject for reducing the individual risk of the subject for developing a cardiovascular disease comprises that the subject receives an assessment about his or her physical fitness, preferably wherein the assessment is given to the subject by a percent value or by defining a status of fitness as being unchanged, decreased or increased.

In a preferred embodiment of the method of the present invention, the individual physical activity program is established by altering the duration, intensity, number of repetitions or number of sessions of a physical activity or the overall combination of different physical activities.

The present invention also relates to the use of at least one circulating miRNA in any of the methods according to the present invention, wherein preferably the at least one miRNA is selected from the group consisting of hsa-miRNA-1, hsa-miRNA-24, hsa-miRNA-96, hsa-miRNA-143, hsa-miRNA-98, hsa-miRNA-125a, hsa-miRNA-132, and any combination, sub-combination, portion or fragment thereof.

In another preferred embodiment of the use of the present invention, the at least one miRNA is selected from the group consisting of hsa-miRNA-1, hsa-miRNA-143, hsa-miRNA-24, hsa-miRNA-96, and any combination, sub-combination, portion or fragment thereof.

In a further preferred embodiment of the use of the present invention, the at least one miRNA is selected from the group consisting of hsa-miRNA-24, hsa-miRNA-96, and any combination, sub-combination, portion or fragment thereof.

### BRIEF DESCRIPTION OF THE FIGURES

FIG. 1 shows the mean miRNA-1 blood concentration before and after 4 minutes of high-intensity interval training. “Rest” means that blood was sampled before the start of the acute exercise session, while “Post.-Ex.” means that blood was sampled immediately after the acute exercise session.

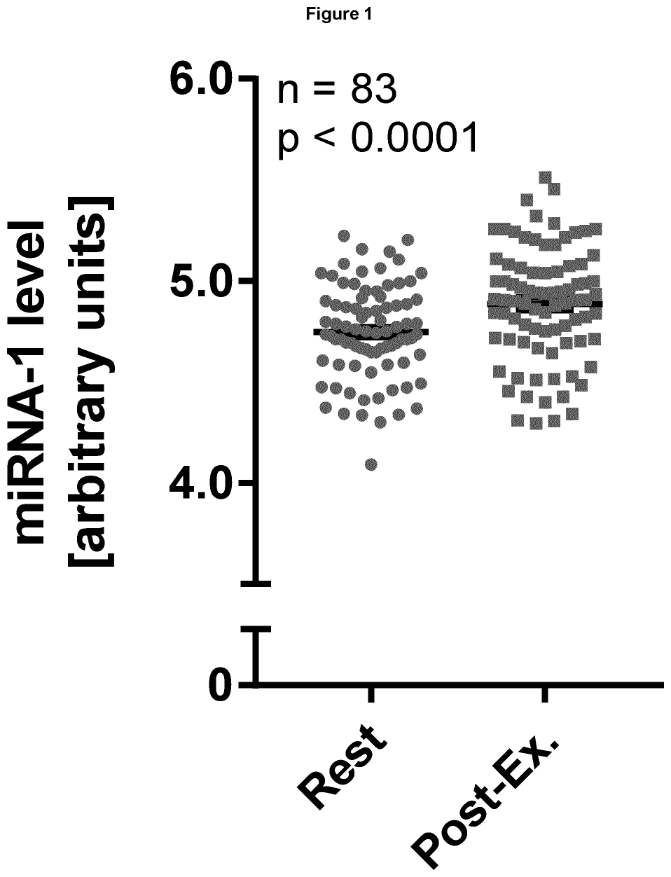

FIG. 2 shows intra-individual changes in miRNA-1 blood concentration in response to 4 minutes of high-intensity interval training. “Rest” means blood was sampled before the start of the acute exercise session, while “Post.-Ex.” means that blood was sampled immediately after the acute exercise session.

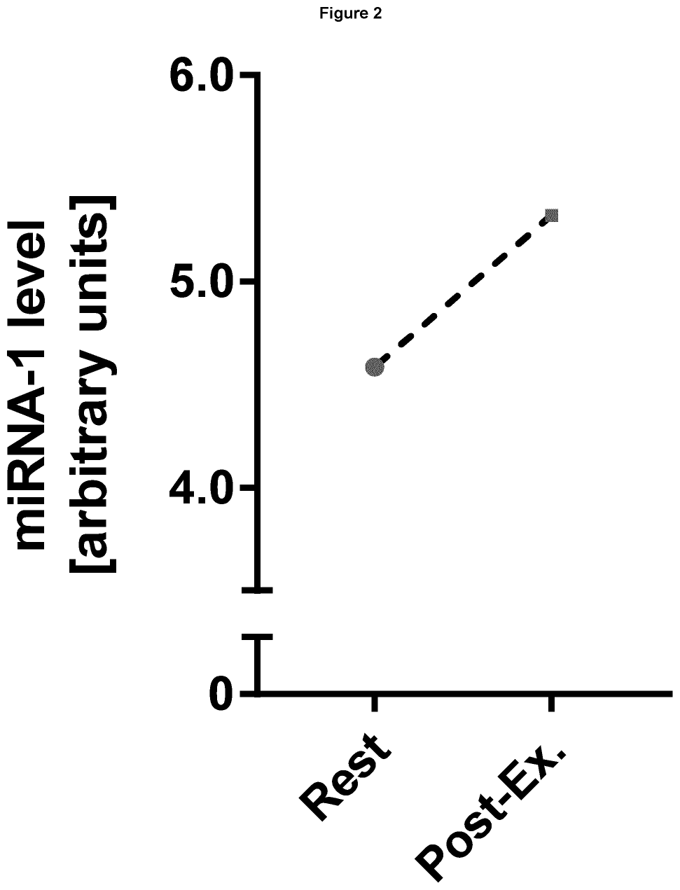

FIG. 3 shows intra-individual changes in miRNA-1 blood concentration during 18 minutes of incremental running exercise. The miRNA-1 blood concentration in a subject before a 4-week training period is shown. A constant drop in miRNA-1 blood concentration was observed with increasing running speed. Blood was sampled at the start of the exercise, during the exercise (after 3 minutes of running at the indicated running speed) and after 3 minutes of recovery (R3). The vertical dotted line indicates the individual lactate threshold of the individual subject, marking the aerobic part of the exercise (left from line; individual low intensity) and the anaerobic part of the exercise (right from line, individual moderate to high intensity).

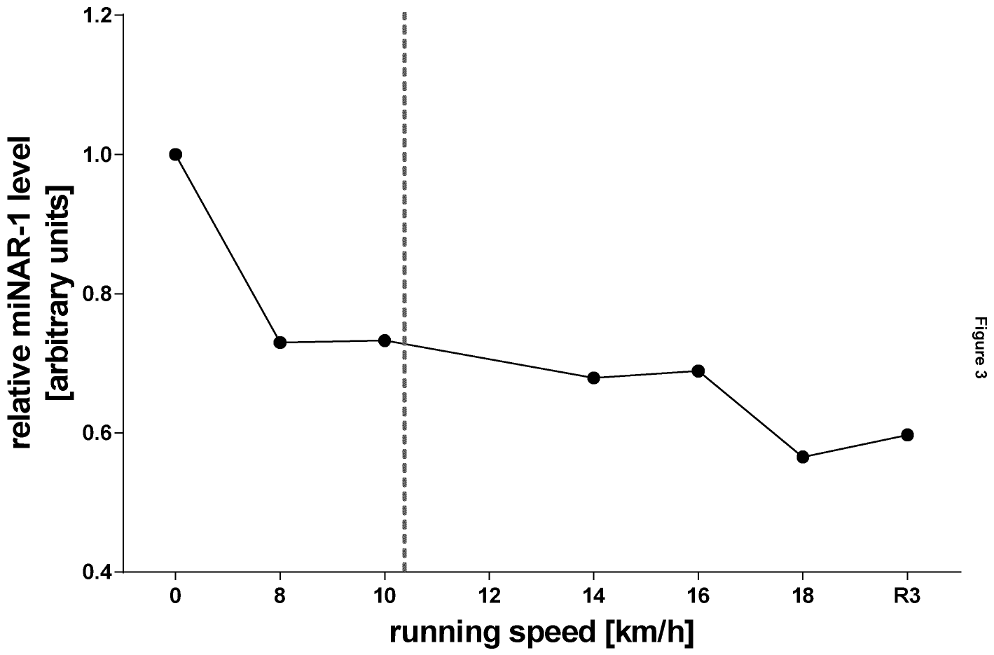

FIG. 4 shows intra-individual changes in miRNA-1 blood concentration during 18 minutes of incremental running exercise. The miRNA-1 blood concentration in a subject (same individual subject as in FIG. 3) after a 4-week training period (2 sessions per week, 4 minutes of high-intensity interval training) is shown. The miRNA-1 blood concentration increased with increasing running speed. Blood was sampled at the start of the exercise, during the exercise (after 3 minutes of running at the indicated running speed) and after 3 minutes of recovery (R3). The vertical dotted line indicates the individual lactate threshold of the individual, marking the aerobic part of the exercise (left from line; individual low intensity) and the anaerobic part of the exercise (right from line, individual moderate to high intensity).

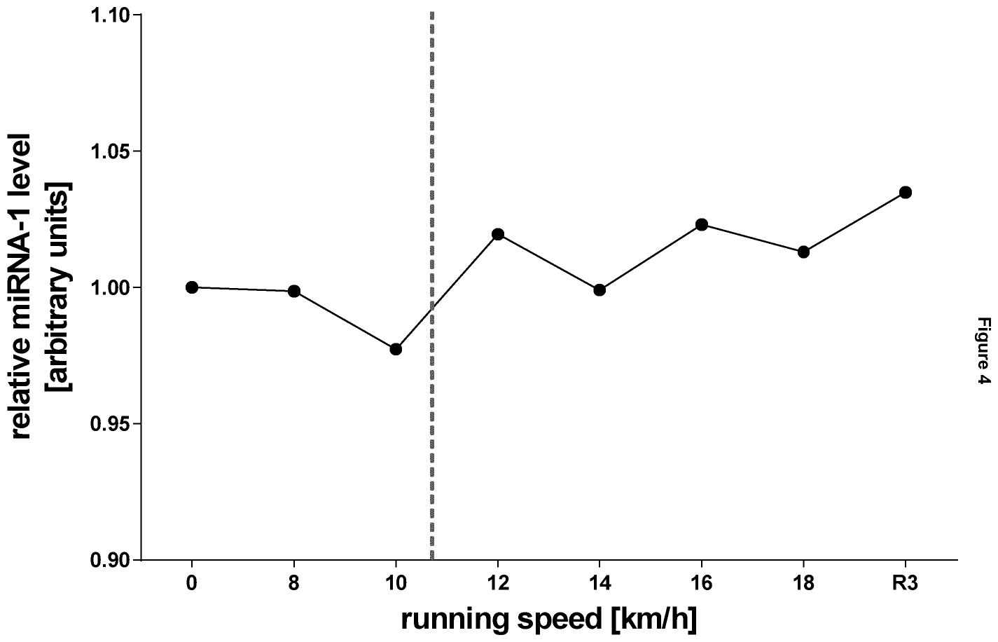

FIG. 5 shows investigation of the physiological mechanism of miRNA-1 elevation during exercise. The miRNA-1 concentration was detected to be elevated in the incubation medium of endothelial cells kept under elevated shear stress (30 dyn/cm² vs. control, 60 minutes). During exercise, haemodynamic forces of the blood stream on the vessel wall increased with increasing exercise intensity. Hymodynamic forces on the vessel endothelium (the inner cell layer at the luminal side) include shear stress induced by the lateral stream of the blood. This condition can be simulated using an in vitro model of endothelial cells kept under different shear rates. In this model, endothelial cells, which can secrete miRNAs into the blood stream upon stimulation, were incubated in a rheometer to mimic shear stress for 60 minutes. The miRNA concentration was determined in the cellular medium.

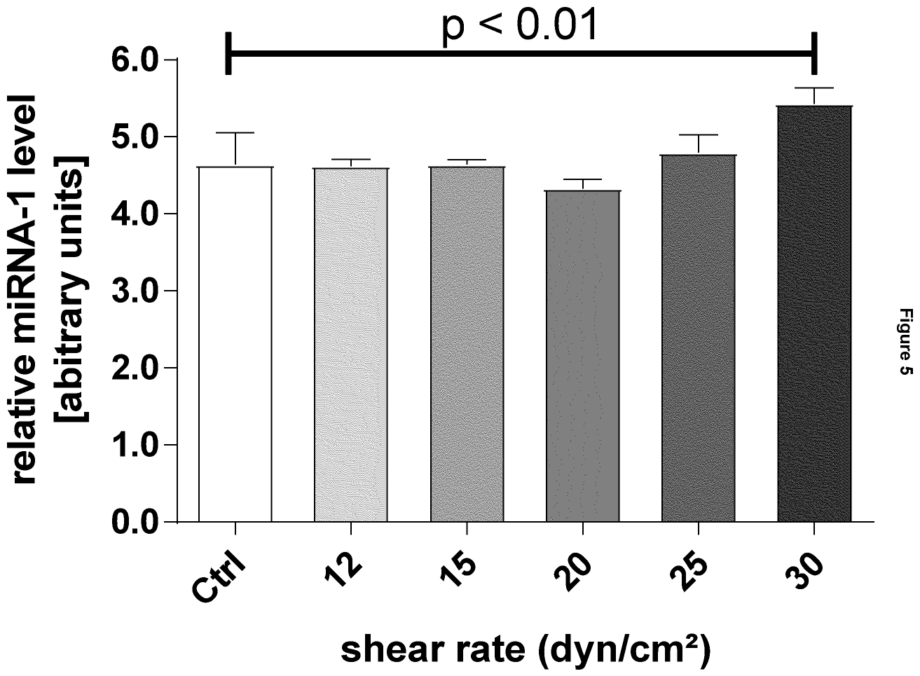

FIG. 6 shows correlation of a combined score of miRNA-24, miRNA-96 and miRNA-143 blood concentration levels with exercise intensity (analysis includes data of 47 individuals for each parameter). Higher miRNA blood levels were observed in individuals with higher blood lactate concentration (a marker of exercise intensity). Blood miRNA and lactate concentration were determined after a session of high-intensity running exercise.

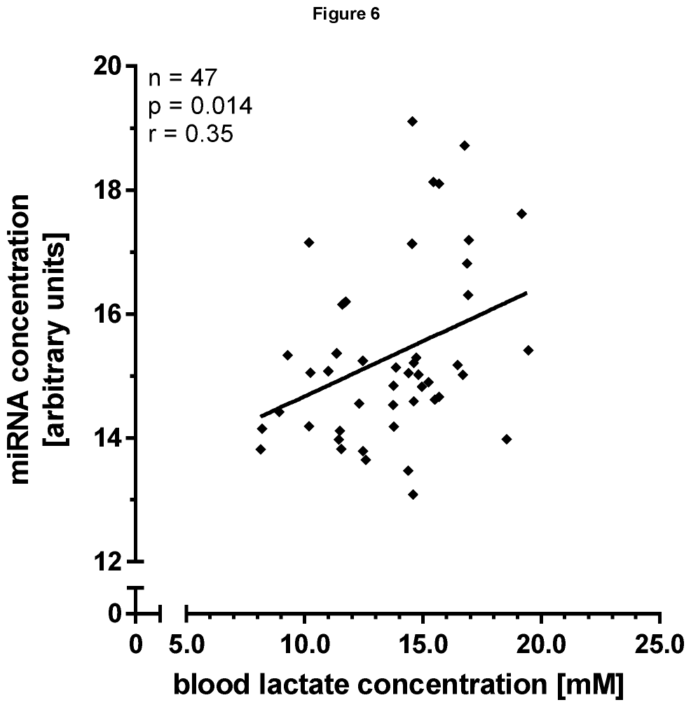

FIG. 7 shows mean miRNA-125a blood concentration before and after 4 minutes of high-intensity interval training. “Rest” means that blood was sampled before the start of the acute exercise session, while “Post.-Ex.” means that blood was sampled immediately after the acute exercise session. “Baseline” means that acute elevation of miRNA-125a blood levels is detected in individuals before regular exercise training. “Post-Training” means that after 4 weeks of regular exercise training (2 sessions per week, 4 minutes of high-intensity interval training) an acute elevation of miRNA-125a blood concentration levels was also observed. The mean miRNA-125a resting level was not significantly elevated after regular exercise training. ****, p<0.0001; **, p<0.01; ns, not significant.

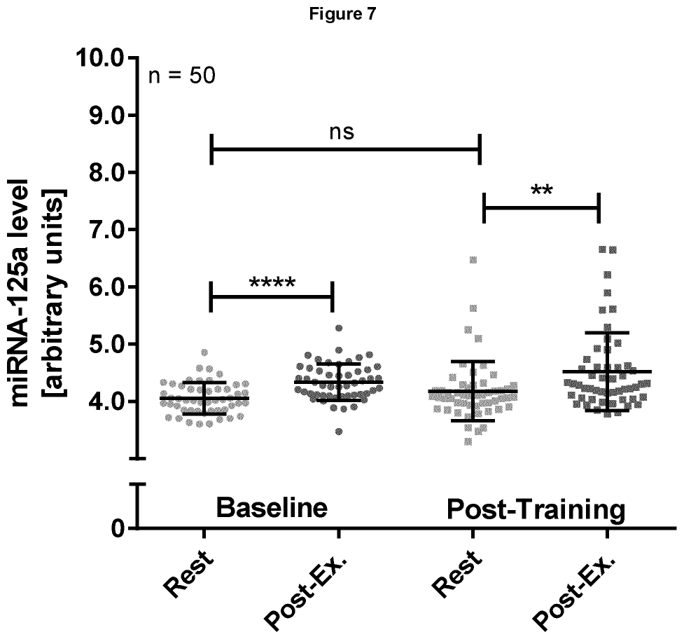

FIG. 8 shows an individual miRNA-125a training profile showing intra-individual changes in miRNA-125a blood concentration of a training responder. “Rest” means that blood was sampled before the start of the acute exercise session, while “Post.-Ex.” means that blood was sampled immediately after the acute exercise session. “Baseline” means that acute elevation of miRNA-125a blood concentration levels was detected before regular exercise training. “Post-Training” shows an acute elevation after 4 weeks of regular exercise training (2 sessions per week, 4 minutes of high-intensity interval training).

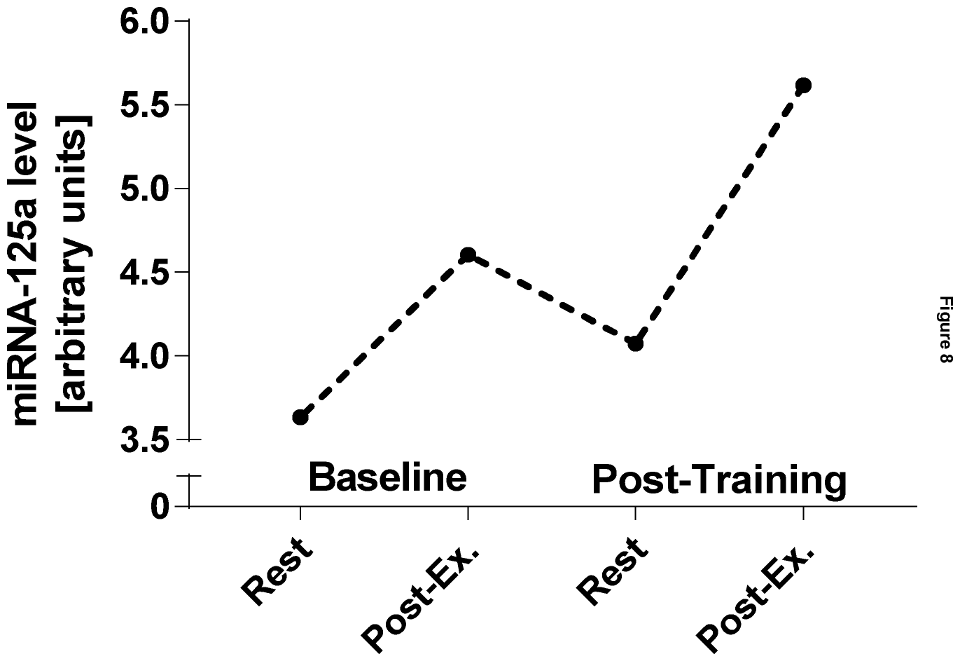

FIG. 9 shows investigation on the physiological mechanism of miRNA-125a elevation during exercise. It was observed by the inventors of the present invention that miRNA-125a concentration is elevated in the incubation medium of endothelial cells kept under elevated shear stress (30 dyn/cm² vs. control, 60 minutes). During exercise, haemodynamic forces of the blood stream on the vessel wall increased with increasing exercise intensity. Hymodynamic forces on the vessel endothelium (the inner cell layer at the luminal side) include shear stress induced by the lateral stream of the blood. This condition can be simulated using an in vitro model of endothelial cells kept under different shear rates. In this model, endothelial cells, which can secrete miRNAs into the blood stream upon stimulation, were incubated in a rheometer to mimic shear stress for 60 minutes. The miRNA concentration was then determined in the cellular medium.

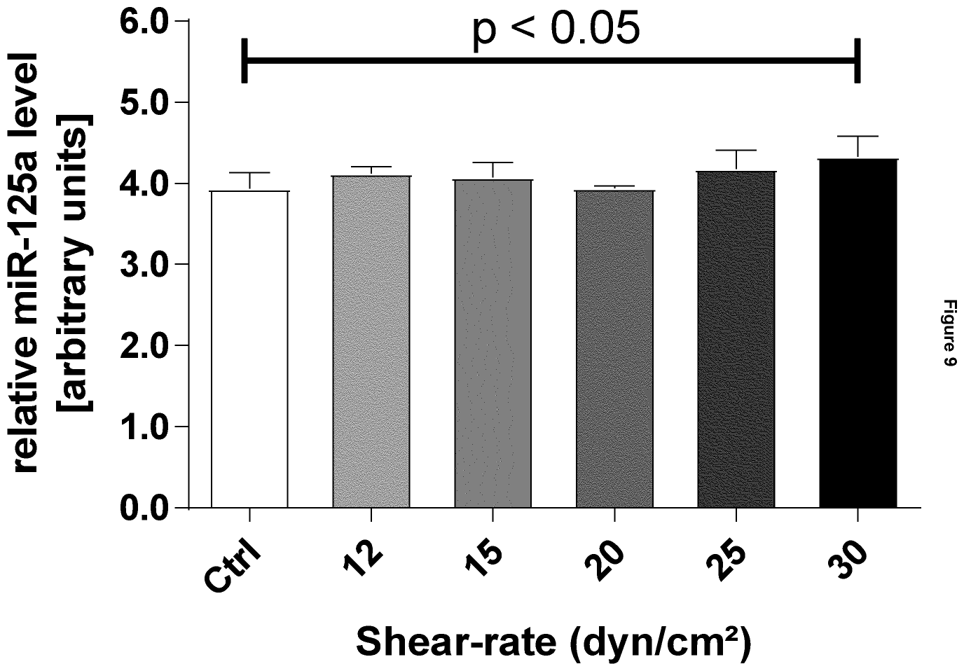

FIG. 10 shows mean miRNA-98 blood concentration before and after 4 minutes of high-intensity interval training. “Rest” means that blood was sampled before the start of the acute exercise session, while “Post.-Ex.” means that blood was sampled immediately after the acute exercise session. “Baseline” means that acute elevation of miRNA-98 blood levels was detected in subjects before regular exercise training. “Post-Training” means that after 4 weeks of regular exercise training (2 sessions per week, 4 minutes of high-intensity interval training), an acute elevation of miRNA-98 blood concentration levels was also observed. The miRNA-98 resting level was not significantly elevated after regular exercise training. ****, p<0.0001; ***, p<0.001; ns, not significant.

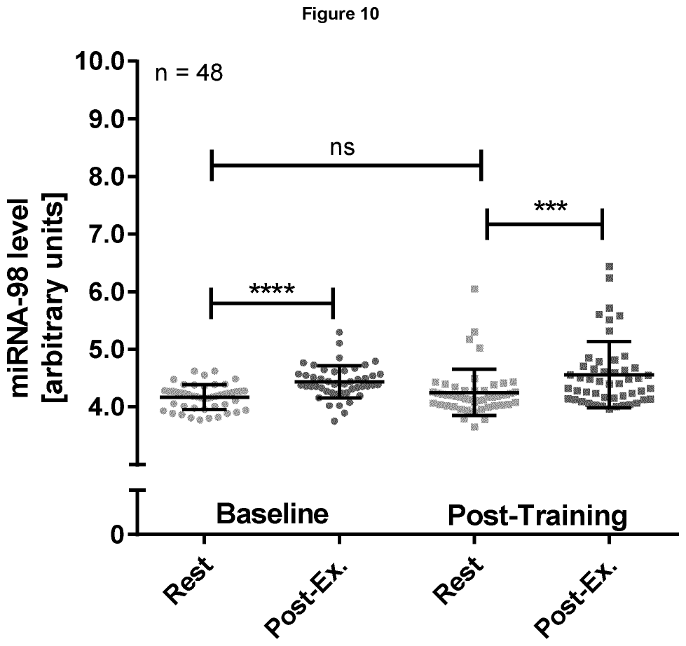

FIG. 11 shows an individual miRNA-98 training profile showing intra-individual changes in miRNA-98 blood concentration of a training responder. “Rest” means that blood was sampled before the start of the acute exercise session, while “Post.-Ex.” means that blood was sampled immediately after the acute exercise session. “Baseline” means that acute elevation of miRNA-98 blood levels was detected before regular exercise training. “Post-Training” means that acute elevation after 4 weeks of regular exercise training (2 sessions per week, 4 minutes of high-intensity interval training) was observed.

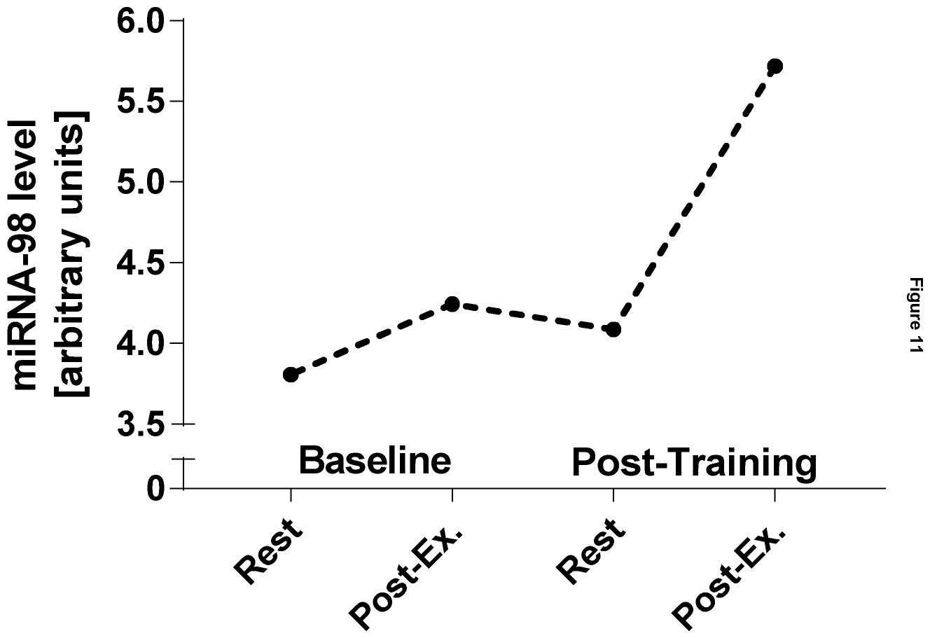

FIG. 12 shows investigation on the physiological mechanism of miRNA-98 elevation during exercise. The miRNA-98 concentration was elevated in the incubation medium of endothelial cells kept under elevated shear stress (30 dyn/cm² vs. control, 60 minutes). During exercise, haemodynamic forces of the blood stream on the vessel wall increased with increasing exercise intensity. Hymodynamic forces on the vessel endothelium (the inner cell layer at the luminal side) include shear stress induced by the lateral stream of the blood. This condition can be simulated using an in vitro model of endothelial cells kept under different shear rates. In this model, endothelial cells, which can secrete miRNAs into the blood stream upon stimulation, were incubated in a rheometer to mimic shear stress for 60 minutes. The miRNA concentration was determined in the cellular medium.

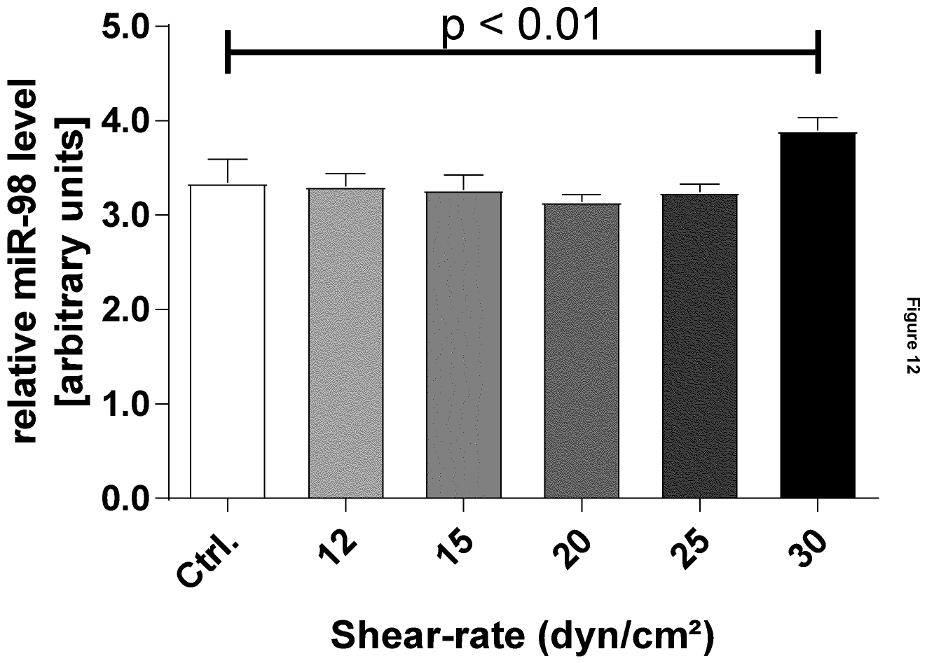

FIG. 13 shows mean miRNA-132 blood concentration before and after moderate-intensity training. “Rest” means that blood was sampled before the start of the acute exercise session, while “Post.-Ex.” means that blood was sampled immediately after the acute exercise session. “Baseline” means that no acute change of miRNA-132 blood levels was detected in subjects before regular exercise training. “Post-Training” means that after 4 weeks of regular exercise training (3 sessions per week, 25 minutes of moderate-intensity training at ˜75% of maximal heart rate), an acute reduction of miRNA-132 blood levels was observed. The mean miRNA-132 resting level was elevated after regular exercise training. ****, p<0.0001; *, p<0.05; ns, not significant.

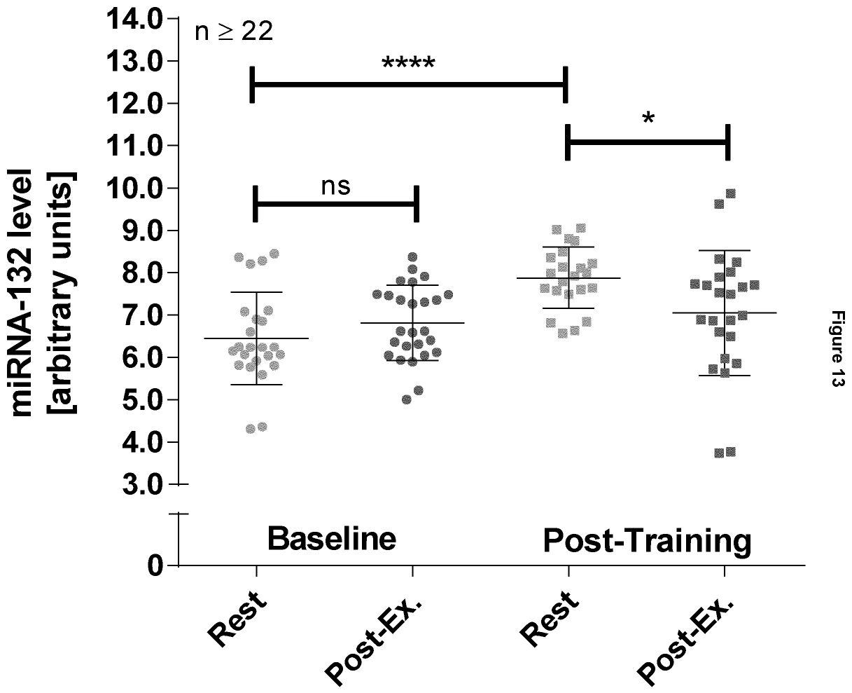

FIG. 14 shows an individual miRNA-132 training profile showing intra-individual changes in miRNA-132 blood concentration of a training responder. “Rest” means that blood was sampled before the start of the acute exercise session, while “Post.-Ex.” means that blood was sampled immediately after the acute exercise session. “Baseline” shows no acute elevation of miRNA-132 blood concentration levels detected before regular exercise training. Post-training shows acute reduction after 4 weeks of regular exercise training (3 sessions per week, 25 minutes of moderate-intensity training at ˜75% of maximal heart rate).

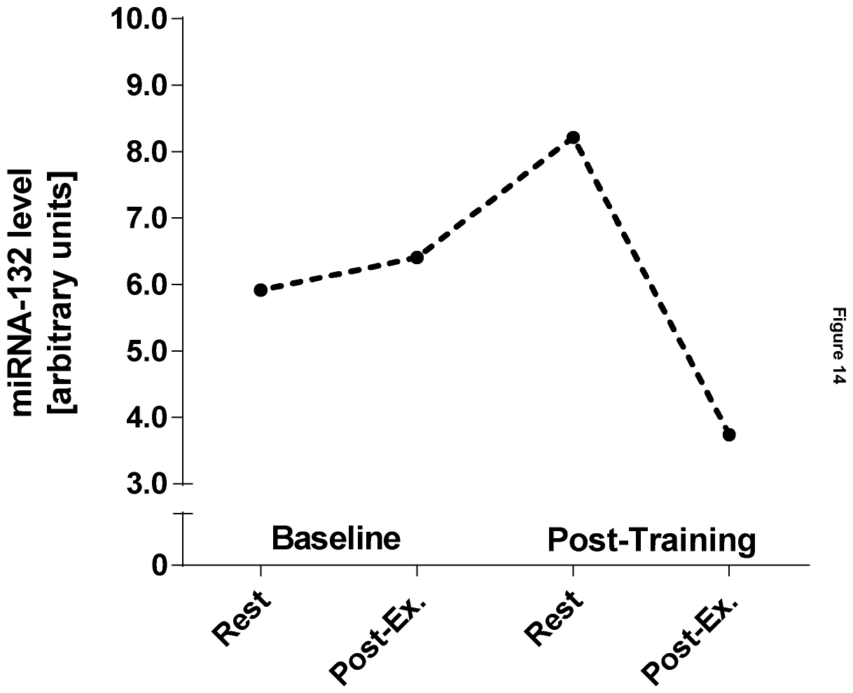

FIG. 15 shows the mean miRNA-132 blood concentration before and after high-intensity interval training. “Rest” means that blood was sampled before the start of the acute exercise session. “During” means that blood was sampled after 4 min of training. “Post.-Ex.” means that blood was sampled immediately after the acute exercise session. Baseline shows no acute change of miRNA-132 blood concentration levels detected in subjects before regular exercise training. Post-training shows, after 4 weeks of regular exercise training (4-7 minutes of high-intensity interval training), an acute reduction of miRNA-132 blood concentration levels. The mean miRNA-132 concentration resting level was not significantly affected. ***, p<0.001; *, p<0.05; ns, not significant.

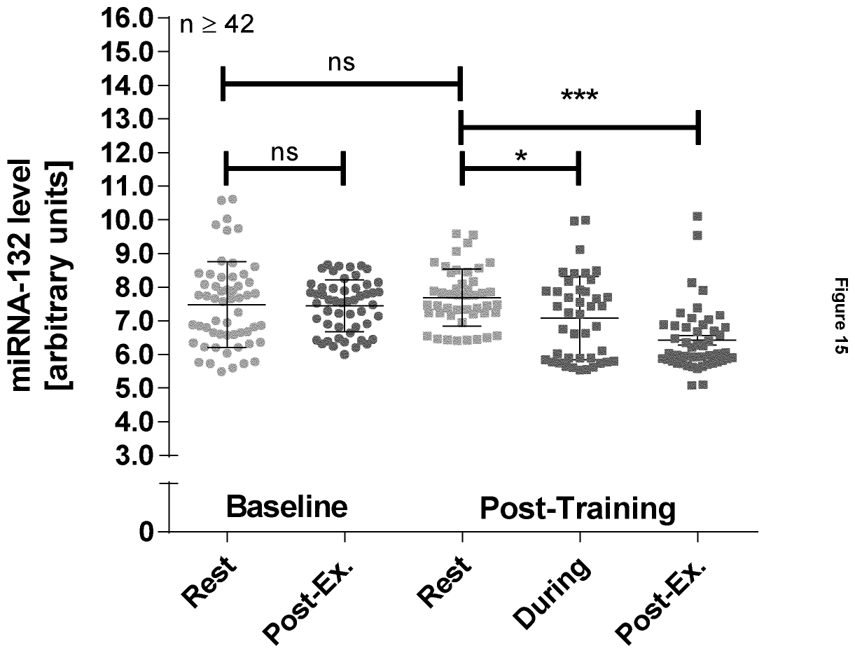

FIG. 16 shows an individual miRNA-132 training profile showing intra-individual changes in miRNA-132 blood concentration of a training responder. “Rest” means that blood was sampled before the start of the acute exercise session. “During” means that blood was sampled after 4 min of training. “Post.-Ex.” means that blood was sampled immediately after the acute exercise session. “Baseline” shows no acute elevation of miRNA-132 blood levels detected before regular exercise training. “Post-Training” shows acute reduction after 4 weeks of regular exercise training (4-7 minutes of high-intensity interval training).

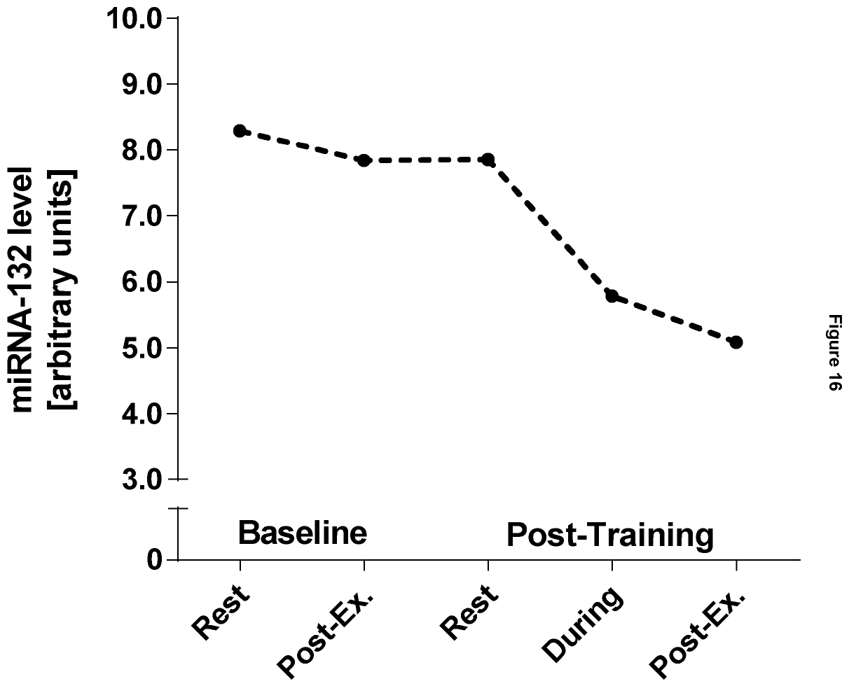

FIG. 17 shows that miRNA-96 blood concentration correlates with microvascular measures. Elevated miRNA-96 blood concentration levels were observed in subjects with better microvascular function. Microvascular function was determined by analysis of perfused boundary region of vessels. Lowered perfused boundary region indicated improved microvascular function. miRNA blood concentration was determined before and after 4 weeks of regular exercise training (2 sessions per week, 4 minutes of high-intensity interval training).

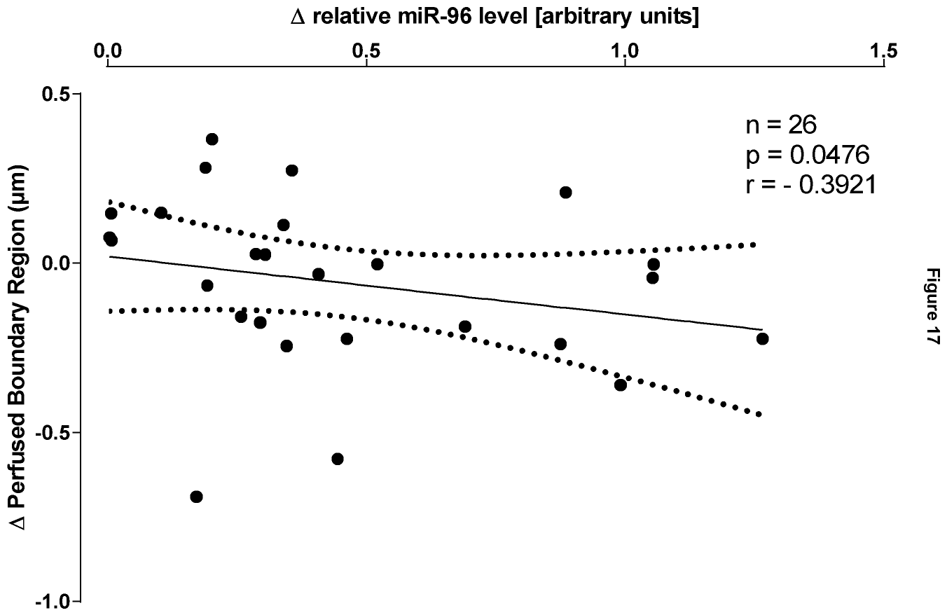

FIG. 18 shows that miRNA-96 blood concentration correlates with microvascular measures. This is shown by receiver operating characteristic curve of microvascular adaptation (decreased perfused boundary region after training intervention) by acute miR-96 increase. “AUC” means the area under the curve with confidence interval.

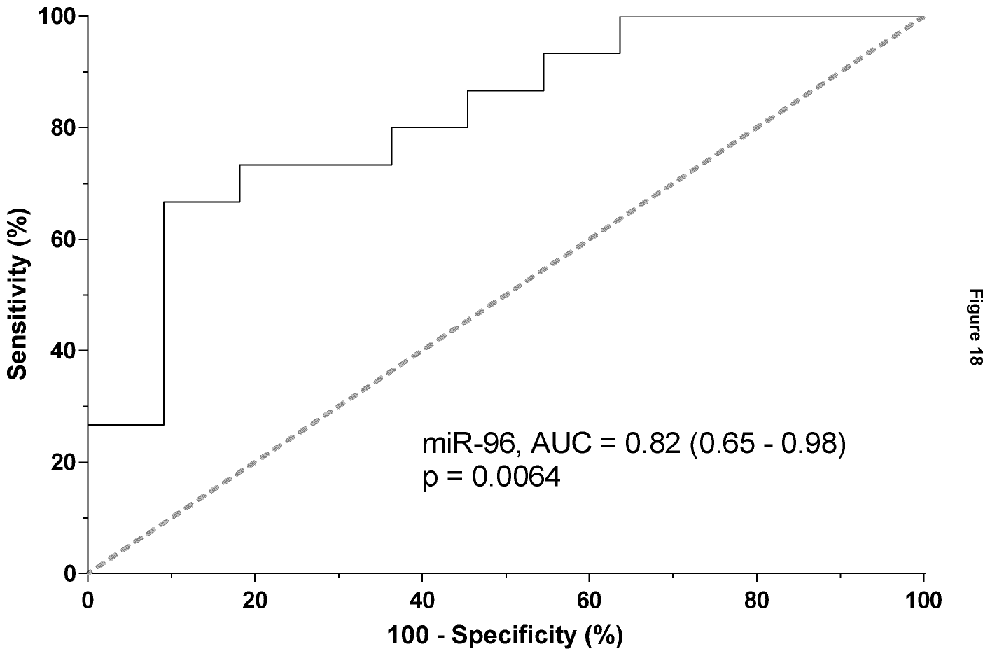

FIG. 19 shows a radar chart (Kiviat diagram) for characterization of acute and sustained exercise training response by combined analysis of miRNA-98 and miRNA-125a. Combined analysis of miRNA-98 and miRNA-125a and their respective changes is indicative for whether a training response is present. The difference between conditions is indicated by non-identical shapes on the upper right (untrained) and lower left (trained) side of the chart. “Rest” means that blood was sampled before the start of an acute exercise session, while “Post.-Ex.” means that blood was sampled immediately after an acute exercise session. “Untrained” means that blood was sampled before regular exercise training. “Trained” means that blood was sampled after 4 weeks of regular exercise training. miRNA levels of more than 50 individuals were analyzed for each time point for chart generation. Data has been normalized to the untrained rest condition for each miRNA and fold changes are given.

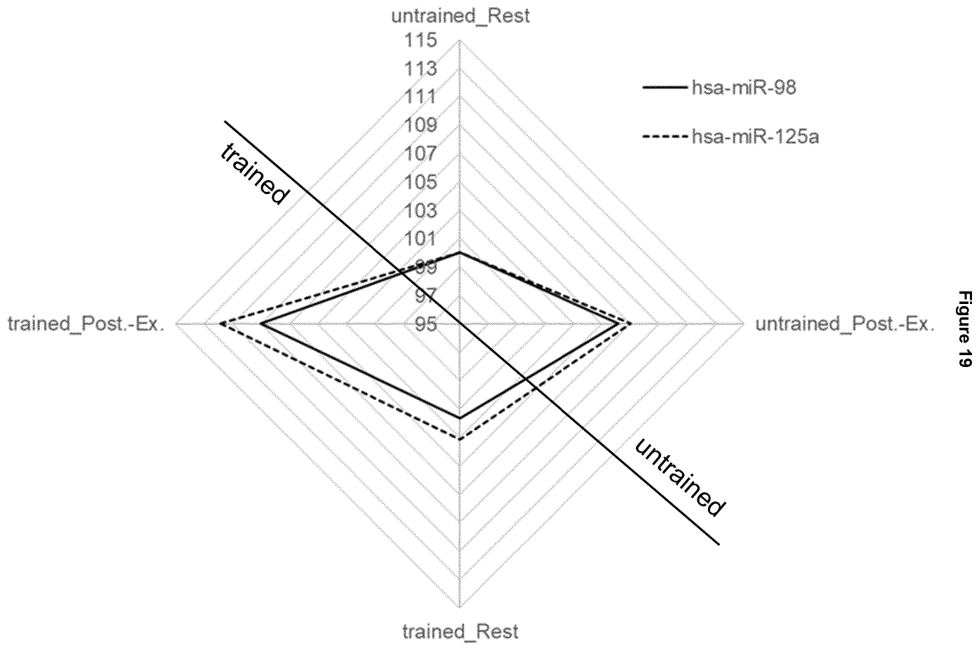

FIG. 20 shows a radar chart (Kiviat diagram) for characterization of acute and sustained exercise training response by combined analysis of miRNA-24 and miRNA-143. Combined analysis of miRNA-24 and miRNA-143 and their respective changes is indicative for whether a training response is present. The difference between conditions is indicated by non-identical shapes on the upper right (untrained) and lower left (trained) side of the chart. “Rest” means that blood was sampled before the start of an acute exercise session, while “Post.-Ex.” means that blood was sampled immediately after an acute exercise session. “Untrained” means that blood was sampled before regular exercise training. “Trained” means that blood was sampled after 4 weeks of regular exercise training. miRNA levels of more than 35 individuals were analyzed for each time point for chart generation. Data has been normalized to the untrained rest condition for each miRNA and fold changes are given.

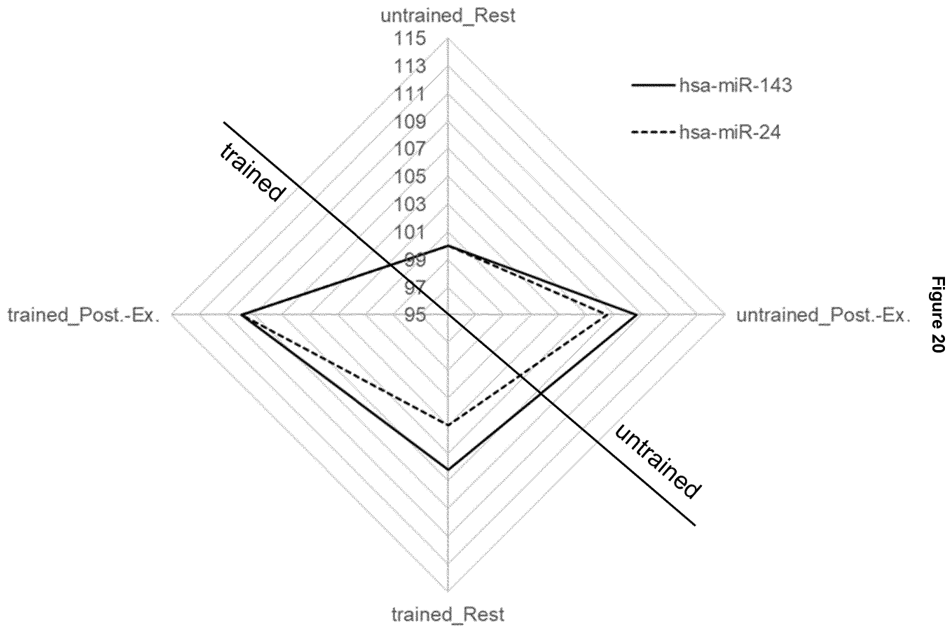

FIG. 21 shows a radar chart (Kiviat diagram) for characterization of acute and sustained exercise training response by combined analysis of miRNA-24, miRNA-143 and miRNA-96-5p. Combined analysis of miRNA-24, miRNA-143 and miRNA-96-5p and their respective changes is indicative for whether a training response is present. The difference between conditions is indicated by non-identical shapes on the upper right (untrained) and lower left (trained) side of the chart. “Rest” means that blood was sampled before the start of an acute exercise session, while “Post.-Ex.” means that blood was sampled immediately after an acute exercise session. “Untrained” means that blood was sampled before regular exercise training. “Trained” means that blood was sampled after 4 weeks of regular exercise training. miRNA levels of more than 35 individuals were analyzed for each time point for chart generation. Data has been normalized to the untrained rest condition for each miRNA and fold changes are given.

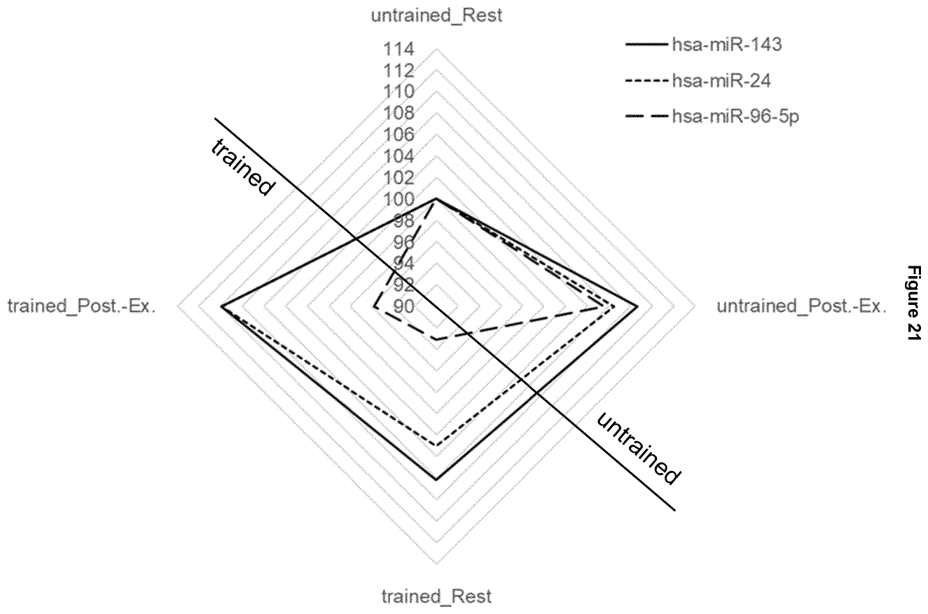

FIG. 22 shows a radar chart (Kiviat diagram) for characterization of acute and sustained exercise training response by combined analysis of miRNA-24, miRNA-143 and miRNA-132. Combined analysis of miRNA-24, miRNA-143 and miRNA-132 and their respective changes is indicative for whether a training response is present. The difference between conditions is indicated by non-identical shapes on the upper right (untrained) and lower left (trained) side of the chart. “Rest” means that blood was sampled before the start of an acute exercise session, while “Post.-Ex.” means that blood was sampled immediately after an acute exercise session. “Untrained” means that blood was sampled before regular exercise training. “Trained” means that blood was sampled after 4 weeks of regular exercise training. miRNA levels of more than 35 individuals were analyzed for each time point for chart generation. Data has been normalized to the untrained rest condition for each miRNA and fold changes are given.

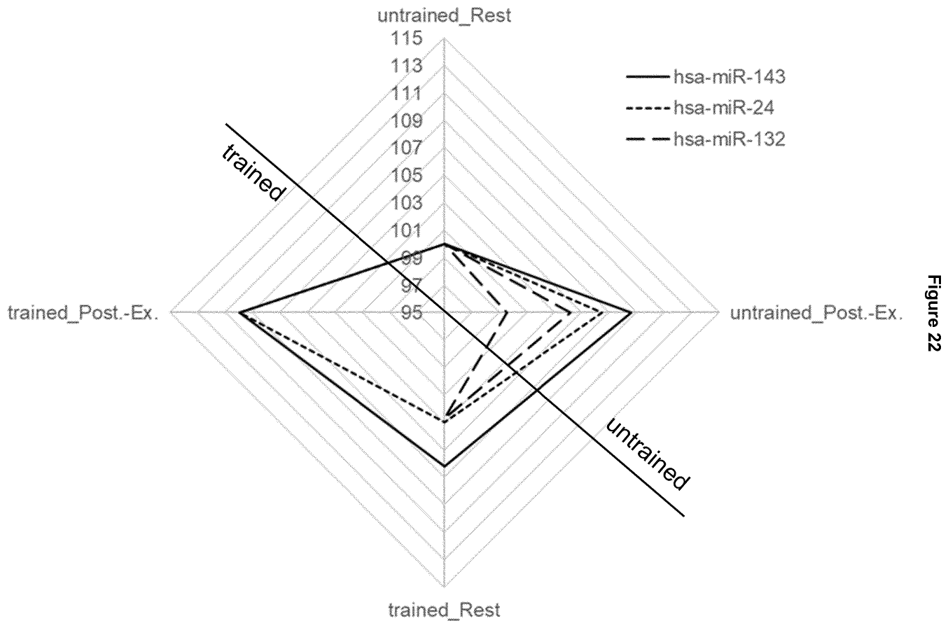

FIG. 23 shows a radar chart (Kiviat diagram) for characterization of acute and sustained exercise training response by combined analysis of miRNA-24, miRNA-96-5p and miRNA-132. Combined analysis of miRNA-24, miRNA-96-5p and miRNA-132 and their respective changes is indicative for whether a training response is present. The difference between conditions is indicated by non-identical shapes on the upper right (untrained) and lower left (trained) side of the chart. “Rest” means that blood was sampled before the start of an acute exercise session, while “Post.-Ex.” means that blood was sampled immediately after an acute exercise session. “Untrained” means that blood was sampled before regular exercise training. “Trained” means that blood was sampled after 4 weeks of regular exercise training. miRNA levels of more than 35 individuals were analyzed for each time point for chart generation. Data has been normalized to the untrained rest condition for each miRNA and fold changes are given.

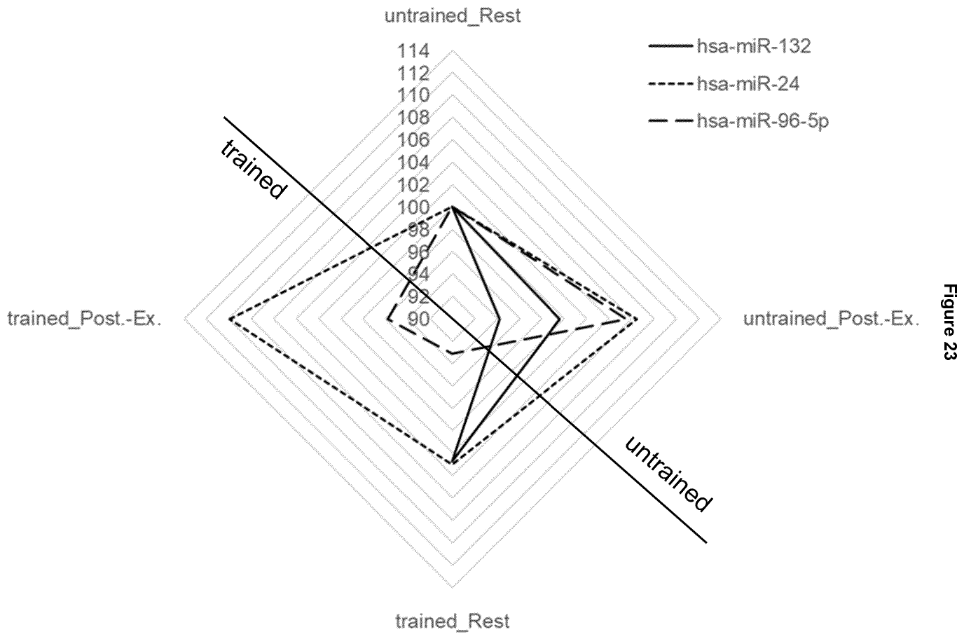

FIG. 24 shows a radar chart (Kiviat diagram) for characterization of acute and sustained exercise training response by combined analysis of miRNA-143, miRNA-96-5p and miRNA-132. Combined analysis of miRNA-143, miRNA-96-5p and miRNA-132 and their respective changes is indicative for whether a training response is present. The difference between conditions is indicated by non-identical shapes on the upper right (untrained) and lower left (trained) side of the chart. “Rest” means that blood was sampled before the start of an acute exercise session, while “Post.-Ex.” means that blood was sampled immediately after an acute exercise session. “Untrained” means that blood was sampled before regular exercise training. “Trained” means that blood was sampled after 4 weeks of regular exercise training. miRNA levels of more than 35 individuals were analyzed for each time point for chart generation. Data has been normalized to the untrained rest condition for each miRNA and fold changes are given.

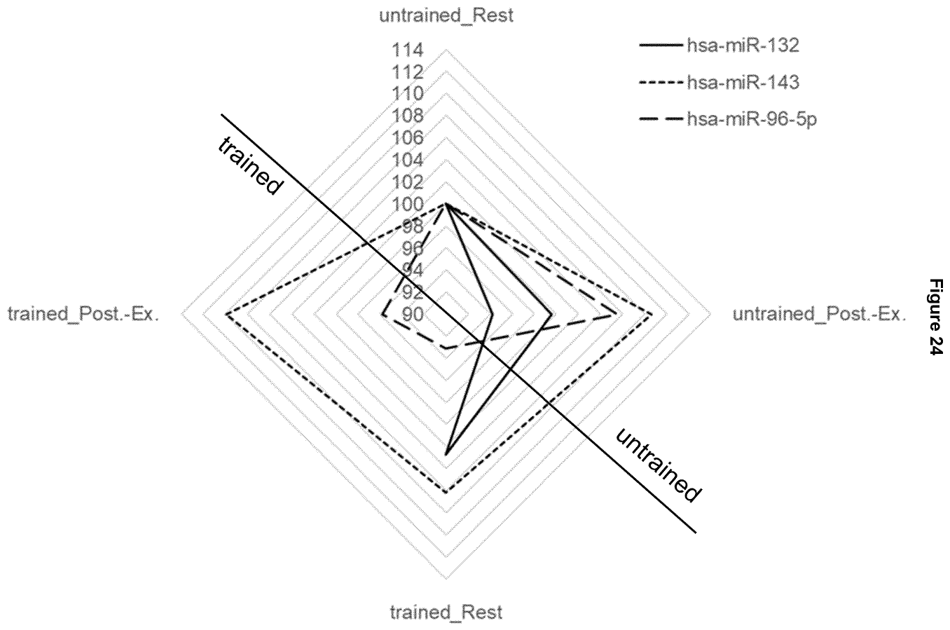

FIG. 25 shows a radar chart (Kiviat diagram) for characterization of acute and sustained exercise training response by combined analysis of miRNA-98 and miRNA-24. Combined analysis of miRNA-98 and miRNA-24 and their respective changes is indicative for whether a training response is present. The difference between conditions is indicated by non-identical shapes on the upper right (untrained) and lower left (trained) side of the chart. “Rest” means that blood was sampled before the start of an acute exercise session, while “Post.-Ex.” means that blood was sampled immediately after an acute exercise session. “Untrained” means that blood was sampled before regular exercise training. “Trained” means that blood was sampled after 4 weeks of regular exercise training. miRNA levels of more than 35 individuals were analyzed for each time point for chart generation. Data has been normalized to the untrained rest condition for each miRNA and fold changes are given.

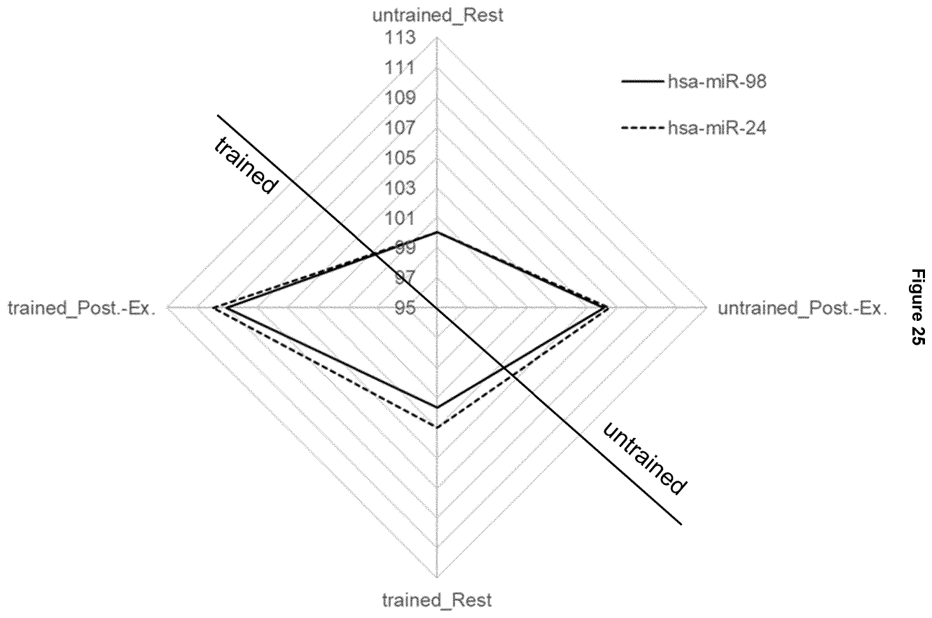

FIG. 26 shows a radar chart (Kiviat diagram) for characterization of acute and sustained exercise training response by combined analysis of miRNA-98 and miRNA-143. Combined analysis of miRNA-98 and miRNA-143 and their respective changes is indicative for whether a training response is present. The difference between conditions is indicated by non-identical shapes on the upper right (untrained) and lower left (trained) side of the chart. “Rest” means that blood was sampled before the start of an acute exercise session, while “Post.-Ex.” means that blood was sampled immediately after an acute exercise session. “Untrained” means that blood was sampled before regular exercise training. “Trained” means that blood was sampled after 4 weeks of regular exercise training. miRNA levels of more than 35 individuals were analyzed for each time point for chart generation. Data has been normalized to the untrained rest condition for each miRNA and fold changes are given.

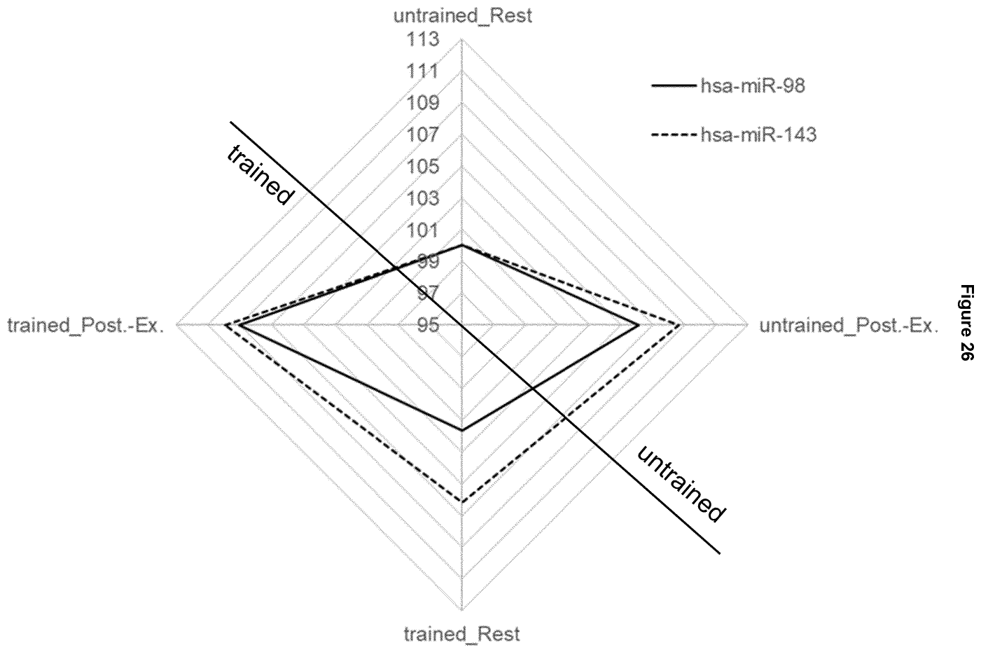

FIG. 27 shows a radar chart (Kiviat diagram) for characterization of acute and sustained exercise training response by combined analysis of miRNA-143, miRNA-98 and miRNA-132. Combined analysis of miRNA-143, miRNA-98 and miRNA-132 and their respective changes is indicative for whether a training response is present. The difference between conditions is indicated by non-identical shapes on the upper right (untrained) and lower left (trained) side of the chart. “Rest” means that blood was sampled before the start of an acute exercise session, while “Post.-Ex.” means that blood was sampled immediately after an acute exercise session. “Untrained” means that blood was sampled before regular exercise training. “Trained” means that blood was sampled after 4 weeks of regular exercise training. miRNA levels of more than 35 individuals were analyzed for each time point for chart generation. Data has been normalized to the untrained rest condition for each miRNA and fold changes are given.

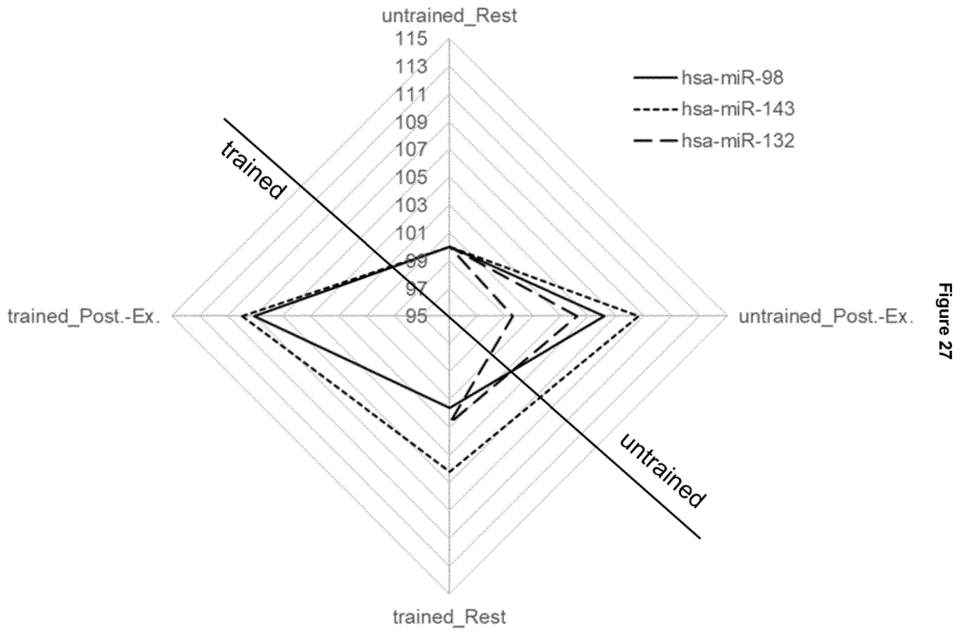

FIG. 28 shows a radar chart (Kiviat diagram) for characterization of acute and sustained exercise training response by combined analysis of miRNA-24, miRNA-98 and miRNA-132. Combined analysis of miRNA-24, miRNA-98 and miRNA-132 and their respective changes is indicative for whether a training response is present. The difference between conditions is indicated by non-identical shapes on the upper right (untrained) and lower left (trained) side of the chart. “Rest” means that blood was sampled before the start of an acute exercise session, while “Post.-Ex.” means that blood was sampled immediately after an acute exercise session. “Untrained” means that blood was sampled before regular exercise training. “Trained” means that blood was sampled after 4 weeks of regular exercise training. miRNA levels of more than 35 individuals were analyzed for each time point for chart generation. Data has been normalized to the untrained rest condition for each miRNA and fold changes are given.

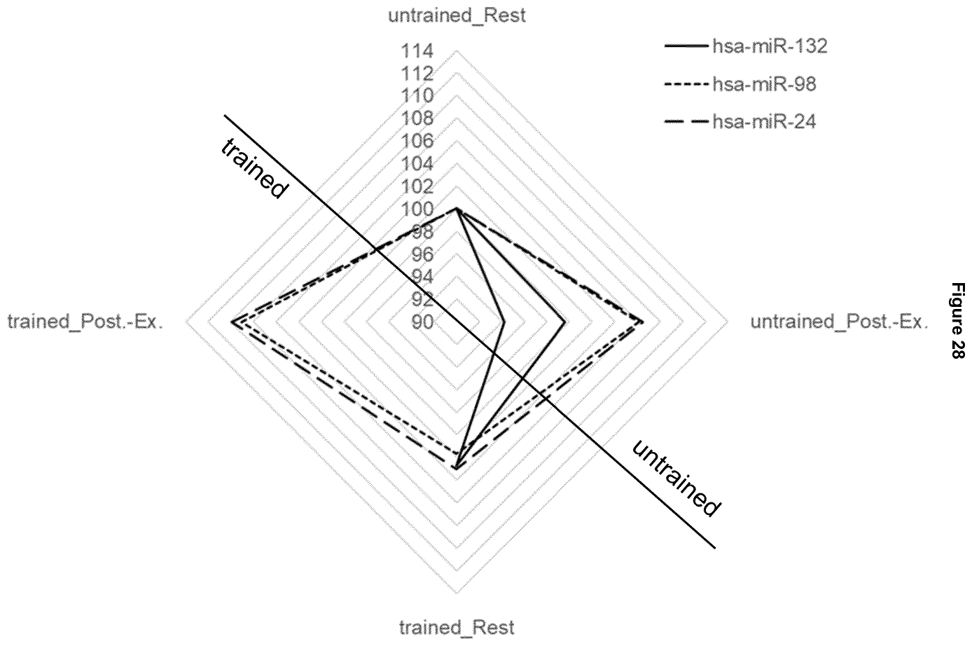

FIG. 29 shows a radar chart (Kiviat diagram) for characterization of acute and sustained exercise training response by combined analysis of miRNA-24 and miRNA-125a. Combined analysis of miRNA-24 and miRNA-125a and their respective changes is indicative for whether a training response is present. The difference between conditions is indicated by non-identical shapes on the upper right (untrained) and lower left (trained) side of the chart. “Rest” means that blood was sampled before the start of an acute exercise session, while “Post.-Ex.” means that blood was sampled immediately after an acute exercise session. “Untrained” means that blood was sampled before regular exercise training. “Trained” means that blood was sampled after 4 weeks of regular exercise training. miRNA levels of more than 35 individuals were analyzed for each time point for chart generation. Data has been normalized to the untrained rest condition for each miRNA and fold changes are given.

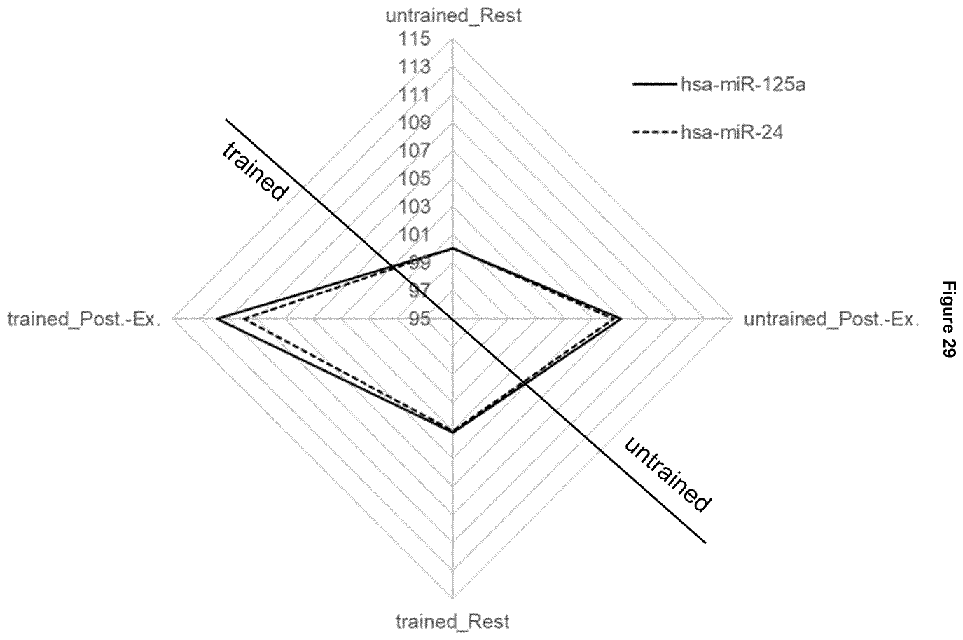

FIG. 30 shows a radar chart (Kiviat diagram) for characterization of acute and sustained exercise training response by combined analysis of miRNA-143, miRNA-125a and miRNA-132. Combined analysis of miRNA-143, miRNA-125a and miRNA-132 and their respective changes is indicative for whether a training response is present. The difference between conditions is indicated by non-identical shapes on the upper right (untrained) and lower left (trained) side of the chart. “Rest” means that blood was sampled before the start of an acute exercise session, while “Post.-Ex.” means that blood was sampled immediately after an acute exercise session. “Untrained” means that blood was sampled before regular exercise training. “Trained” means that blood was sampled after 4 weeks of regular exercise training. miRNA levels of more than 35 individuals were analyzed for each time point for chart generation. Data has been normalized to the untrained rest condition for each miRNA and fold changes are given.

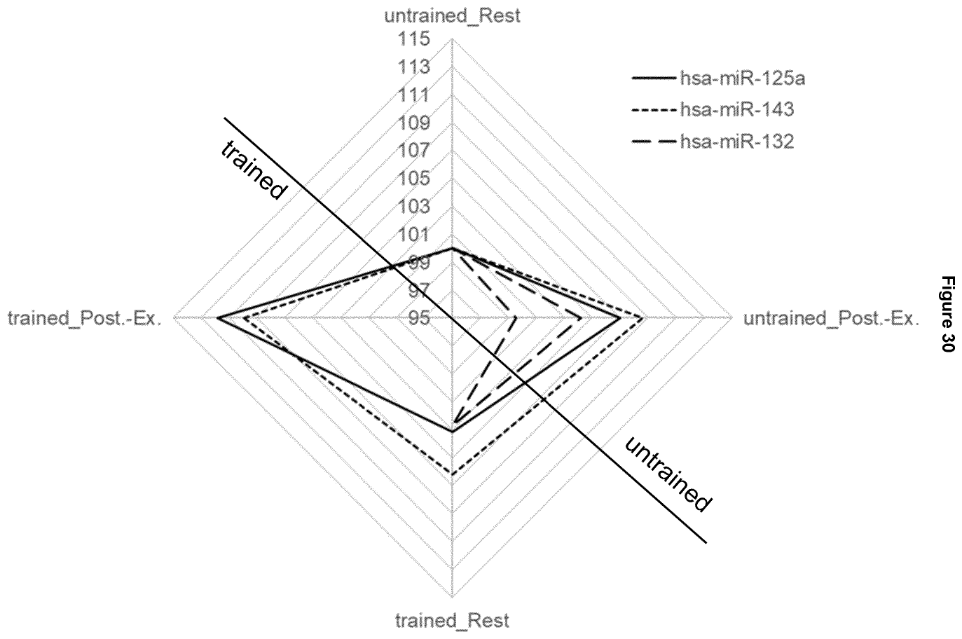

FIG. 31 shows a radar chart (Kiviat diagram) for characterization of acute and sustained exercise training response by combined analysis of miRNA-24, miRNA-125a and miRNA-132. Combined analysis of miRNA-24, miRNA-125a and miRNA-132 and their respective changes is indicative for whether a training response is present. The difference between conditions is indicated by non-identical shapes on the upper right (untrained) and lower left (trained) side of the chart. “Rest” means that blood was sampled before the start of an acute exercise session, while “Post.-Ex.” means that blood was sampled immediately after an acute exercise session. “Untrained” means that blood was sampled before regular exercise training. “Trained” means that blood was sampled after 4 weeks of regular exercise training. miRNA levels of more than 35 individuals were analyzed for each time point for chart generation. Data has been normalized to the untrained rest condition for each miRNA and fold changes are given.

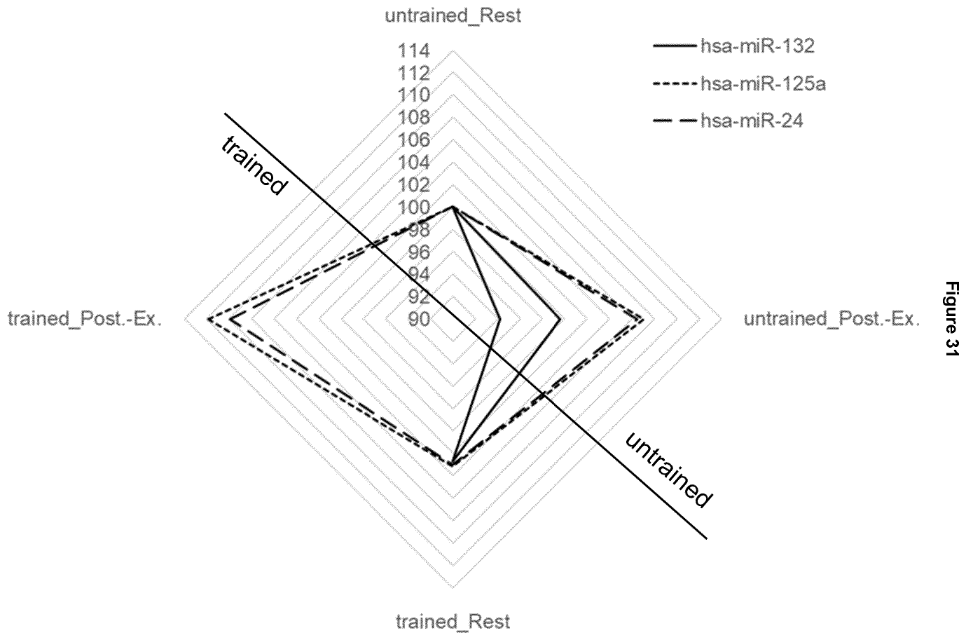

FIG. 32 shows a radar chart (Kiviat diagram) for characterization of acute and sustained exercise training response by combined analysis of miRNA-96-5p and miRNA-98. Combined analysis of miRNA-96-5p and miRNA-98 and their respective changes is indicative for whether a training response is present. The difference between conditions is indicated by non-identical shapes on the upper right (untrained) and lower left (trained) side of the chart. “Rest” means that blood was sampled before the start of an acute exercise session, while “Post.-Ex.” means that blood was sampled immediately after an acute exercise session. “Untrained” means that blood was sampled before regular exercise training. “Trained” means that blood was sampled after 4 weeks of regular exercise training. miRNA levels of more than 35 individuals were analyzed for each time point for chart generation. Data has been normalized to the untrained rest condition for each miRNA and fold changes are given.

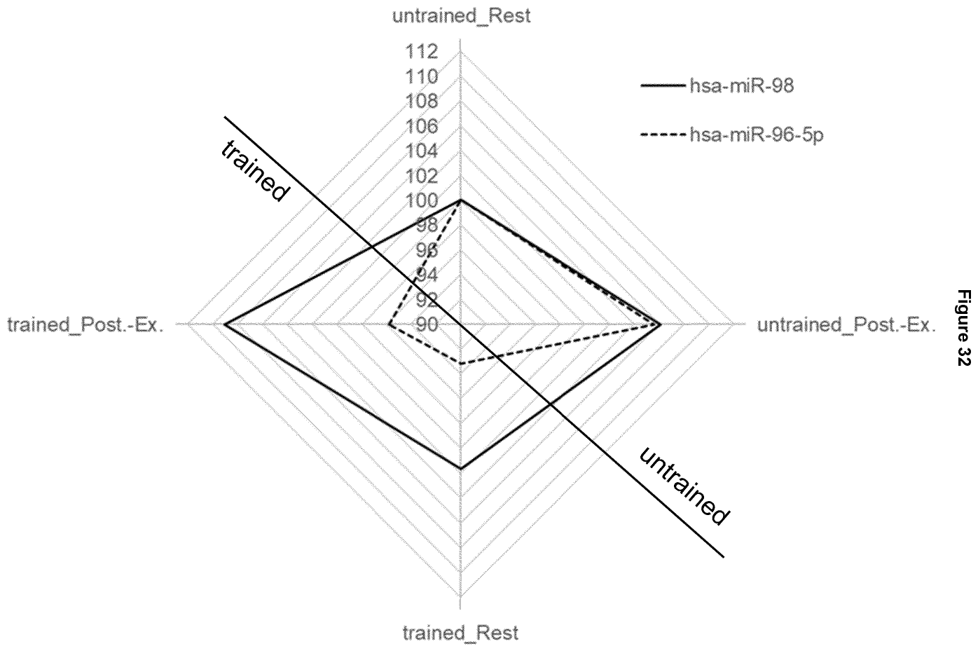

FIG. 33 shows a radar chart (Kiviat diagram) for characterization of acute and sustained exercise training response by combined analysis of miRNA-96-5p, miRNA-125a and miRNA-98. Combined analysis of miRNA-96-5p, miRNA-125a and miRNA-98 and their respective changes is indicative for whether a training response is present. The difference between conditions is indicated by non-identical shapes on the upper right (untrained) and lower left (trained) side of the chart. “Rest” means that blood was sampled before the start of an acute exercise session, while “Post.-Ex.” means that blood was sampled immediately after an acute exercise session. “Untrained” means that blood was sampled before regular exercise training. “Trained” means that blood was sampled after 4 weeks of regular exercise training. miRNA levels of more than 35 individuals were analyzed for each time point for chart generation. Data has been normalized to the untrained rest condition for each miRNA and fold changes are given.

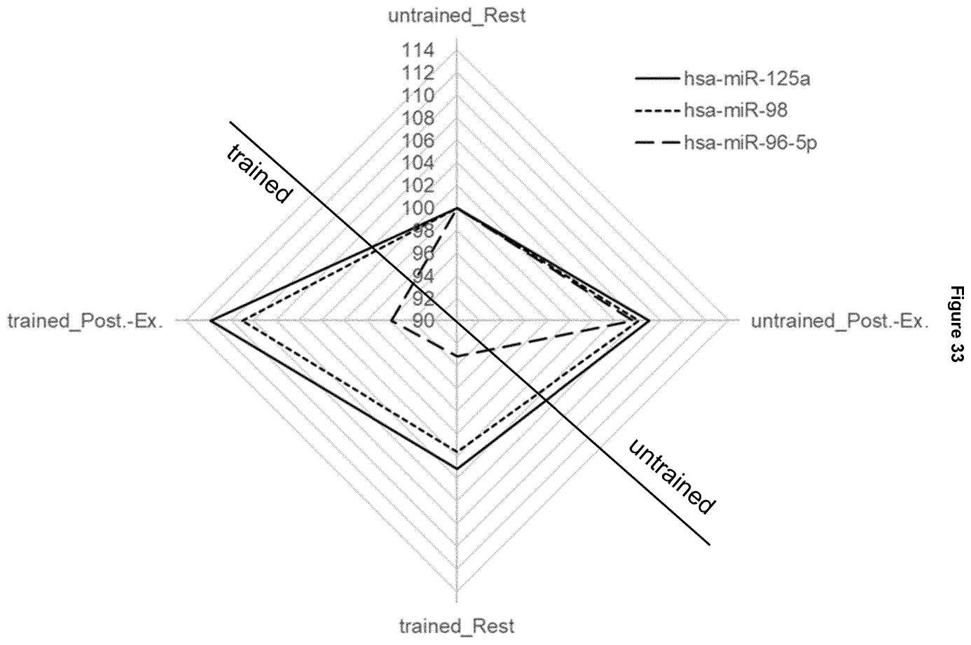

FIG. 34 shows a radar chart (Kiviat diagram) for characterization of acute and sustained exercise training response by combined analysis of miRNA-96-5p, miRNA-24 and miRNA-98. Combined analysis of miRNA-96-5p, miRNA-24 and miRNA-98 and their respective changes is indicative for whether a training response is present. The difference between conditions is indicated by non-identical shapes on the upper right (untrained) and lower left (trained) side of the chart. “Rest” means that blood was sampled before the start of an acute exercise session, while “Post.-Ex.” means that blood was sampled immediately after an acute exercise session. “Untrained” means that blood was sampled before regular exercise training. “Trained” means that blood was sampled after 4 weeks of regular exercise training. miRNA levels of more than 35 individuals were analyzed for each time point for chart generation. Data has been normalized to the untrained rest condition for each miRNA and fold changes are given.

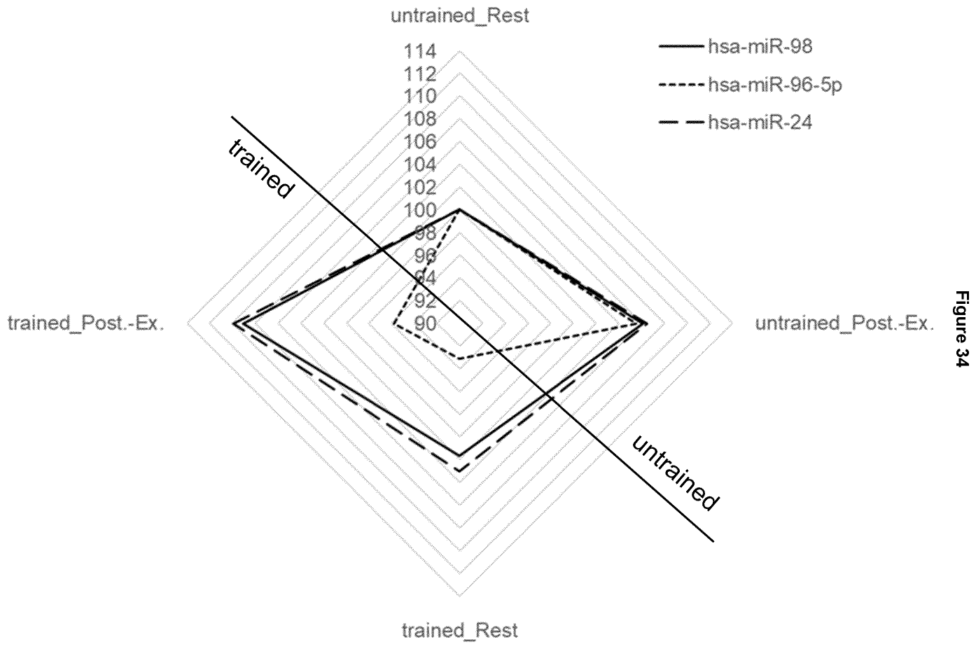

FIG. 35 shows a radar chart (Kiviat diagram) for characterization of acute and sustained exercise training response by combined analysis of miRNA-96-5p, miRNA-143 and miRNA-98. Combined analysis of miRNA-96-5p, miRNA-143 and miRNA-98 and their respective changes is indicative for whether a training response is present. The difference between conditions is indicated by non-identical shapes on the upper right (untrained) and lower left (trained) side of the chart. “Rest” means that blood was sampled before the start of an acute exercise session, while “Post.-Ex.” means that blood was sampled immediately after an acute exercise session. “Untrained” means that blood was sampled before regular exercise training. “Trained” means that blood was sampled after 4 weeks of regular exercise training. miRNA levels of more than 35 individuals were analyzed for each time point for chart generation. Data has been normalized to the untrained rest condition for each miRNA and fold changes are given.

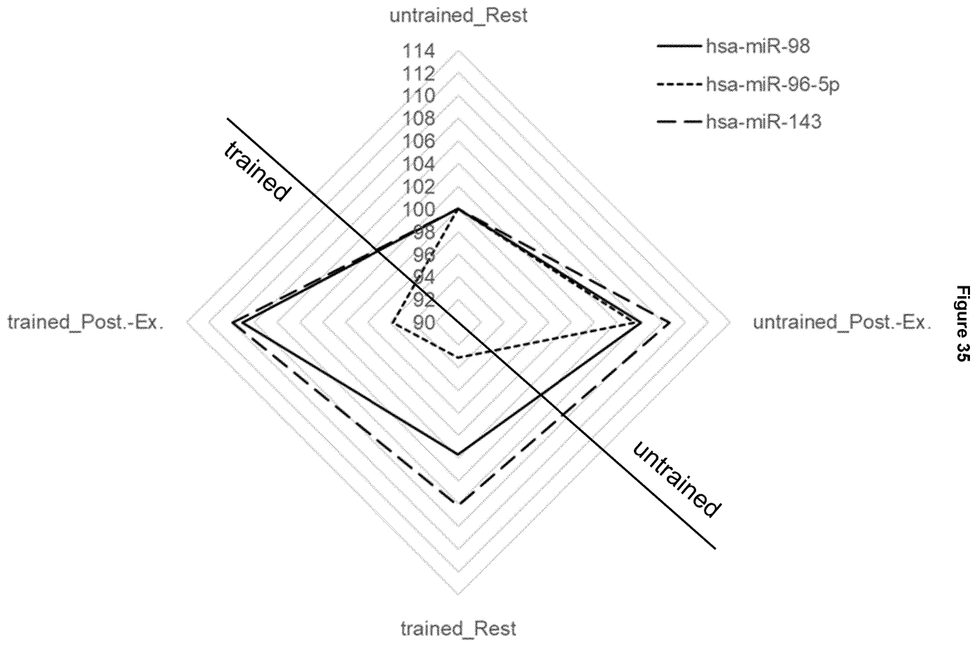

FIG. 36 shows a radar chart (Kiviat diagram) for characterization of acute and sustained exercise training response by combined analysis of miRNA-96-5p and miRNA-143. Combined analysis of miRNA-96-5p and miRNA-143 and their respective changes is indicative for whether a training response is present. The difference between conditions is indicated by non-identical shapes on the upper right (untrained) and lower left (trained) side of the chart. “Rest” means that blood was sampled before the start of an acute exercise session, while “Post.-Ex.” means that blood was sampled immediately after an acute exercise session. “Untrained” means that blood was sampled before regular exercise training. “Trained” means that blood was sampled after 4 weeks of regular exercise training. miRNA levels of more than 35 individuals were analyzed for each time point for chart generation. Data has been normalized to the untrained rest condition for each miRNA and fold changes are given.

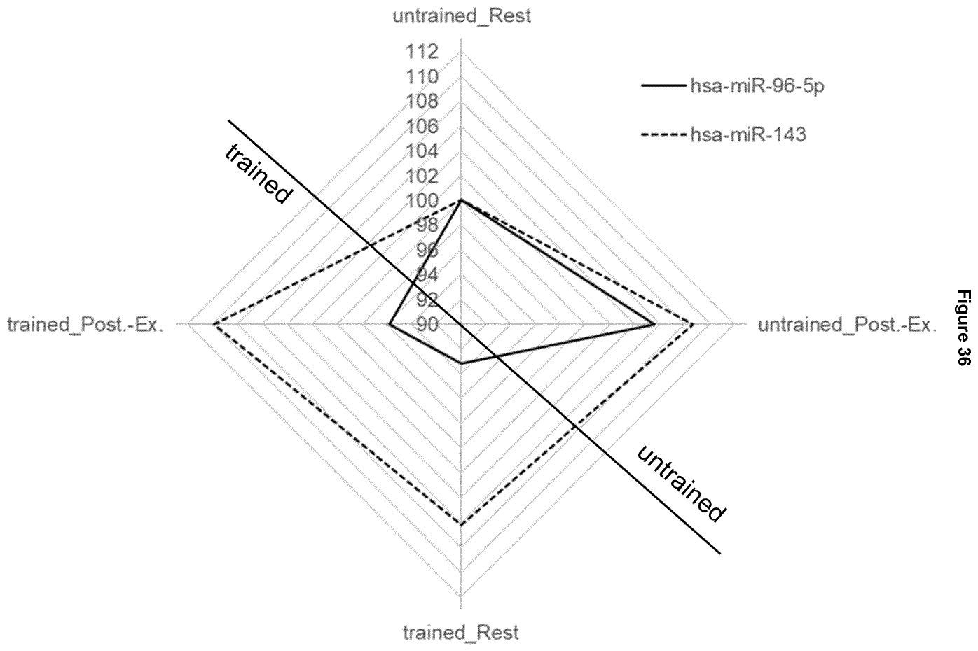

FIG. 37 shows a radar chart (Kiviat diagram) for characterization of acute and sustained exercise training response by combined analysis of miRNA-96-5p, miRNA-143 and miRNA-125a. Combined analysis of miRNA-96-5p, miRNA-143 and miRNA-125a and their respective changes is indicative for whether a training response is present. The difference between conditions is indicated by non-identical shapes on the upper right (untrained) and lower left (trained) side of the chart. “Rest” means that blood was sampled before the start of an acute exercise session, while “Post.-Ex.” means that blood was sampled immediately after an acute exercise session. “Untrained” means that blood was sampled before regular exercise training. “Trained” means that blood was sampled after 4 weeks of regular exercise training. miRNA levels of more than 35 individuals were analyzed for each time point for chart generation. Data has been normalized to the untrained rest condition for each miRNA and fold changes are given.

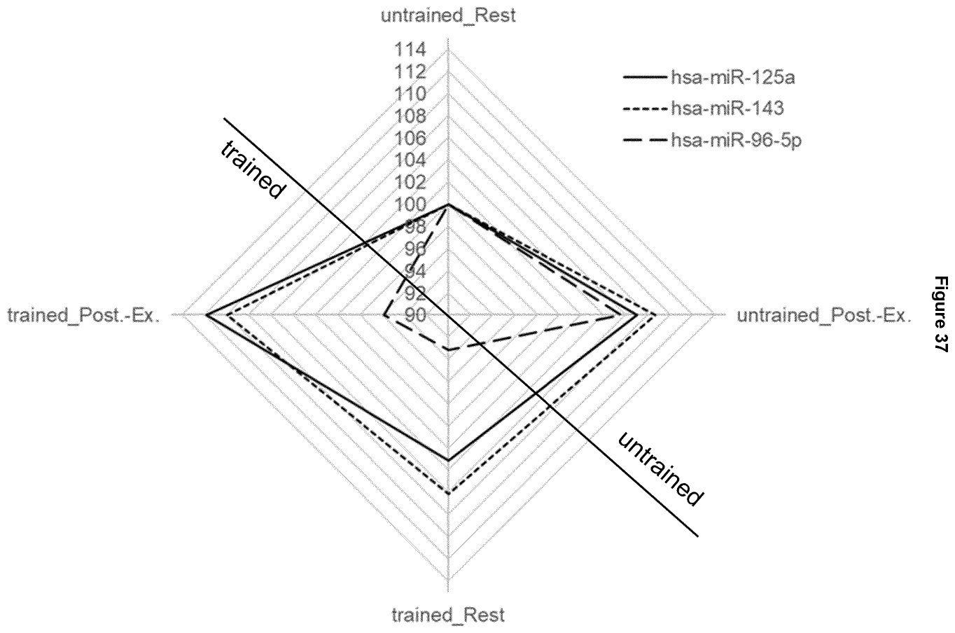

FIG. 38 shows a radar chart (Kiviat diagram) for characterization of acute and sustained exercise training response by combined analysis of miRNA-98, miRNA-143 and miRNA-125a. Combined analysis of miRNA-98, miRNA-143 and miRNA-125a and their respective changes is indicative for whether a training response is present. The difference between conditions is indicated by non-identical shapes on the upper right (untrained) and lower left (trained) side of the chart. “Rest” means that blood was sampled before the start of an acute exercise session, while “Post.-Ex.” means that blood was sampled immediately after an acute exercise session. “Untrained” means that blood was sampled before regular exercise training. “Trained” means that blood was sampled after 4 weeks of regular exercise training. miRNA levels of more than 35 individuals were analyzed for each time point for chart generation. Data has been normalized to the untrained rest condition for each miRNA and fold changes are given.

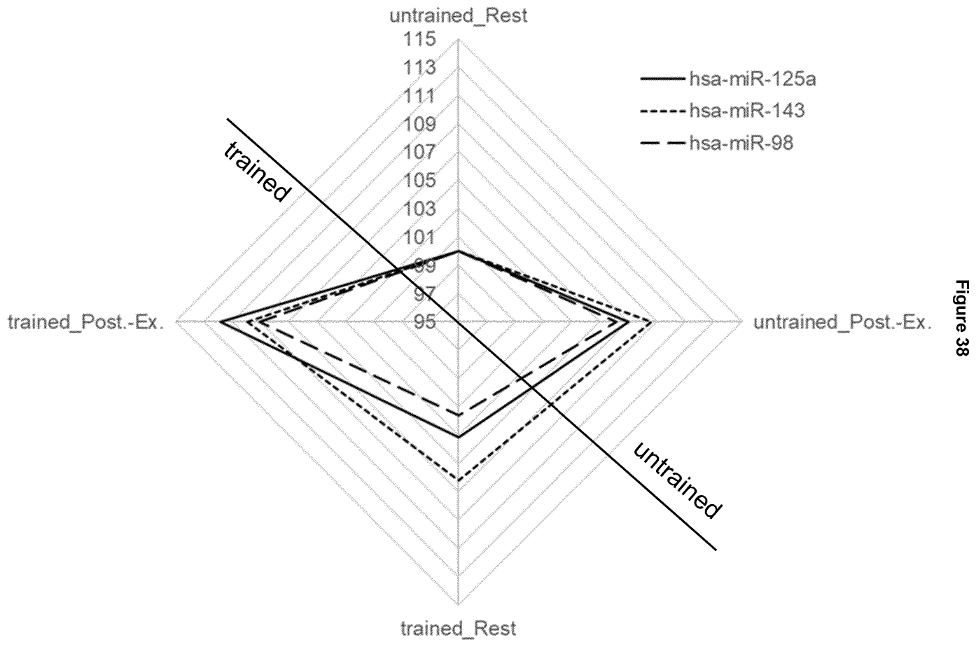

FIG. 39 shows a radar chart (Kiviat diagram) for characterization of acute and sustained exercise training response by combined analysis of miRNA-98, miRNA-24 and miRNA-125a. Combined analysis of miRNA-98, miRNA-24 and miRNA-125a and their respective changes is indicative for whether a training response is present. The difference between conditions is indicated by non-identical shapes on the upper right (untrained) and lower left (trained) side of the chart. “Rest” means that blood was sampled before the start of an acute exercise session, while “Post.-Ex.” means that blood was sampled immediately after an acute exercise session. “Untrained” means that blood was sampled before regular exercise training. “Trained” means that blood was sampled after 4 weeks of regular exercise training. miRNA levels of more than 35 individuals were analyzed for each time point for chart generation. Data has been normalized to the untrained rest condition for each miRNA and fold changes are given.

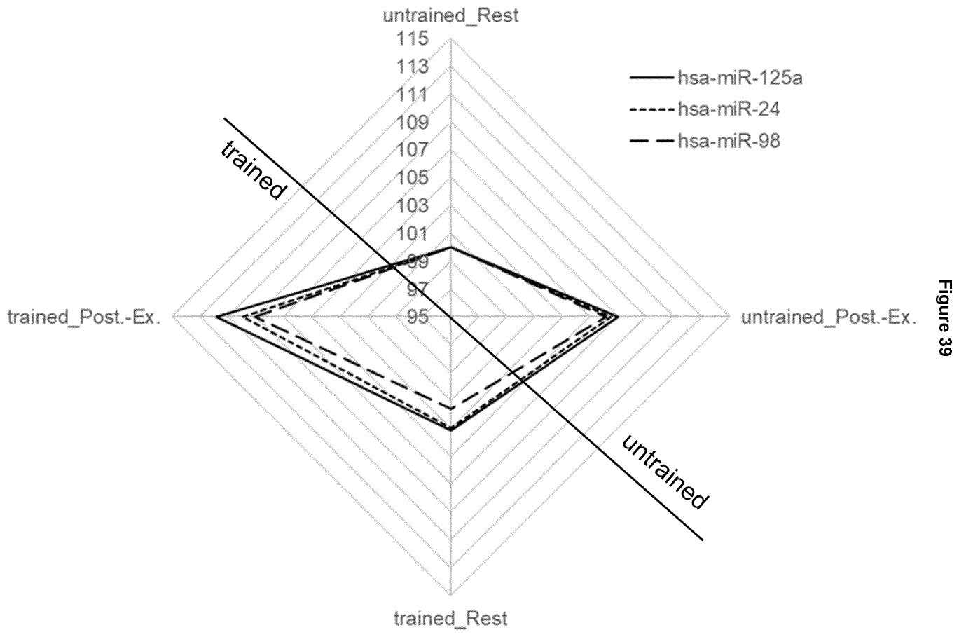

FIG. 40 shows a radar chart (Kiviat diagram) for characterization of acute and sustained exercise training response by combined analysis of miRNA-96-5p, miRNA-24 and miRNA-125a. Combined analysis of miRNA-96-5p, miRNA-24 and miRNA-125a and their respective changes is indicative for whether a training response is present. The difference between conditions is indicated by non-identical shapes on the upper right (untrained) and lower left (trained) side of the chart. “Rest” means that blood was sampled before the start of an acute exercise session, while “Post.-Ex.” means that blood was sampled immediately after an acute exercise session. “Untrained” means that blood was sampled before regular exercise training. “Trained” means that blood was sampled after 4 weeks of regular exercise training. miRNA levels of more than 35 individuals were analyzed for each time point for chart generation. Data has been normalized to the untrained rest condition for each miRNA and fold changes are given.

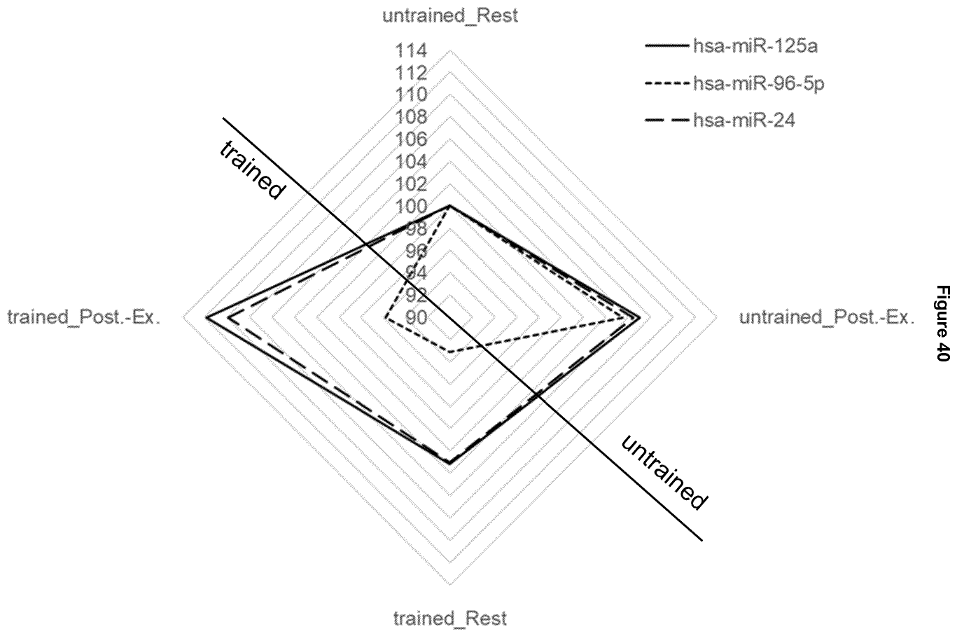

FIG. 41 shows a radar chart (Kiviat diagram) for characterization of acute and sustained exercise training response by combined analysis of miRNA-96-5p and miRNA-125a. Combined analysis of miRNA-96-5p and miRNA-125a and their respective changes is indicative for whether a training response is present. The difference between conditions is indicated by non-identical shapes on the upper right (untrained) and lower left (trained) side of the chart. “Rest” means that blood was sampled before the start of an acute exercise session, while “Post.-Ex.” means that blood was sampled immediately after an acute exercise session. “Untrained” means that blood was sampled before regular exercise training. “trained” means that blood was sampled after 4 weeks of regular exercise training. miRNA levels of more than 50 individuals were analyzed for each time point for chart generation. Data has been normalized to the untrained rest condition for each miRNA and fold changes are given.

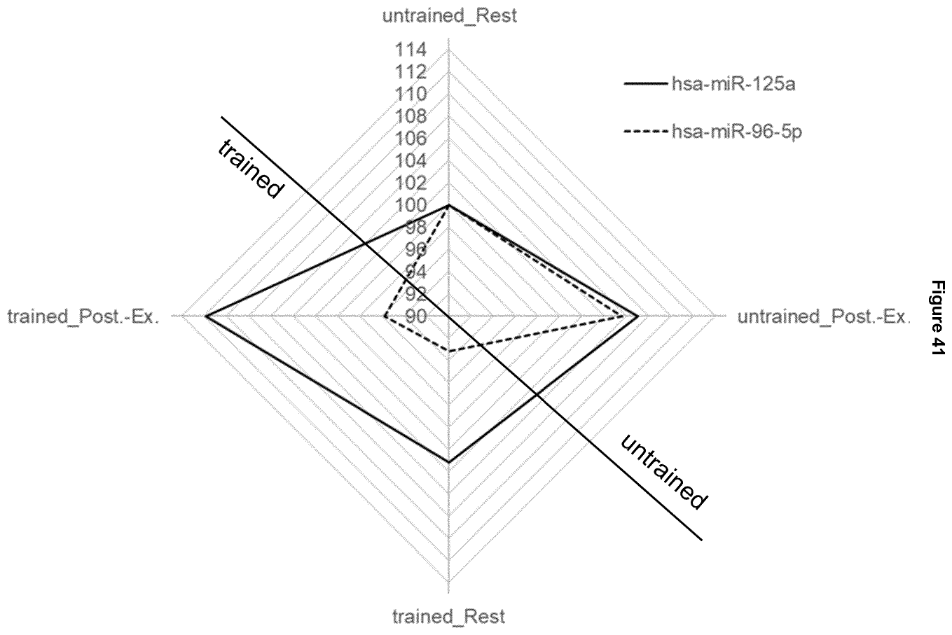

FIG. 42 shows a radar chart (Kiviat diagram) for characterization of acute and sustained exercise training response by combined analysis of miRNA-98, miRNA-132 and miRNA-125a. Combined analysis of miRNA-98, miRNA-132 and miRNA-125a and their respective changes is indicative for whether a training response is present. The difference between conditions is indicated by non-identical shapes on the upper right (untrained) and lower left (trained) side of the chart. “Rest” means that blood was sampled before the start of an acute exercise session, while “Post.-Ex.” means that blood was sampled immediately after an acute exercise session. “Untrained” means that blood was sampled before regular exercise training. “Trained” means that blood was sampled after 4 weeks of regular exercise training. miRNA levels of more than 35 individuals were analyzed for each time point for chart generation. Data has been normalized to the untrained rest condition for each miRNA and fold changes are given.

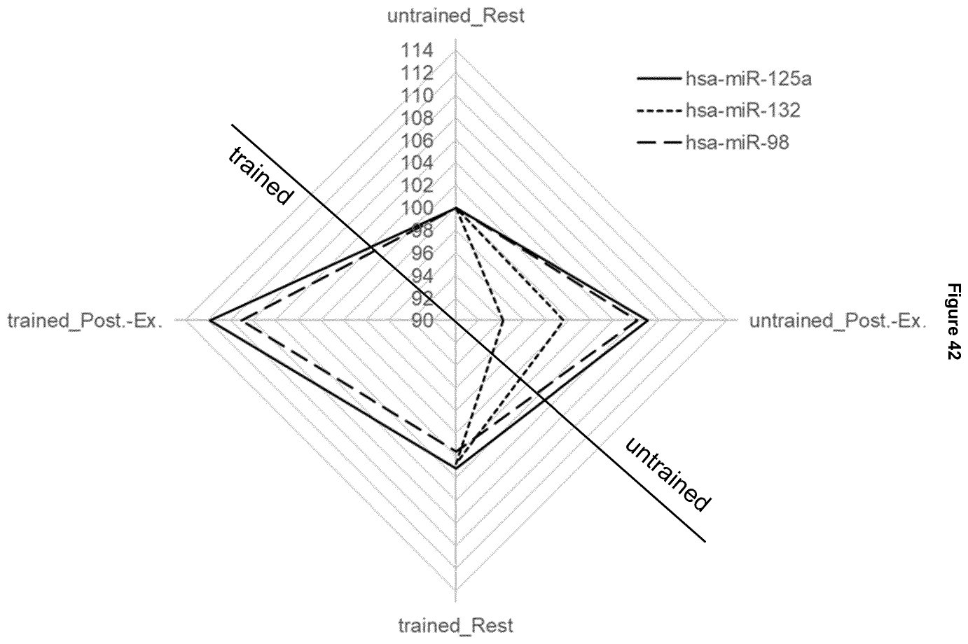

FIG. 43 shows a radar chart (Kiviat diagram) for characterization of acute and sustained exercise training response by combined analysis of miRNA-96-5p, miRNA-132 and miRNA-125a. Combined analysis of miRNA-96-5p, miRNA-132 and miRNA-125a and their respective changes is indicative for whether a training response is present. The difference between conditions is indicated by non-identical shapes on the upper right (untrained) and lower left (trained) side of the chart. “Rest” means that blood was sampled before the start of an acute exercise session, while “Post.-Ex.” means that blood was sampled immediately after an acute exercise session. “Untrained” means that blood was sampled before regular exercise training. “Trained” means that blood was sampled after 4 weeks of regular exercise training. miRNA levels of more than 35 individuals were analyzed for each time point for chart generation. Data has been normalized to the untrained rest condition for each miRNA and fold changes are given.

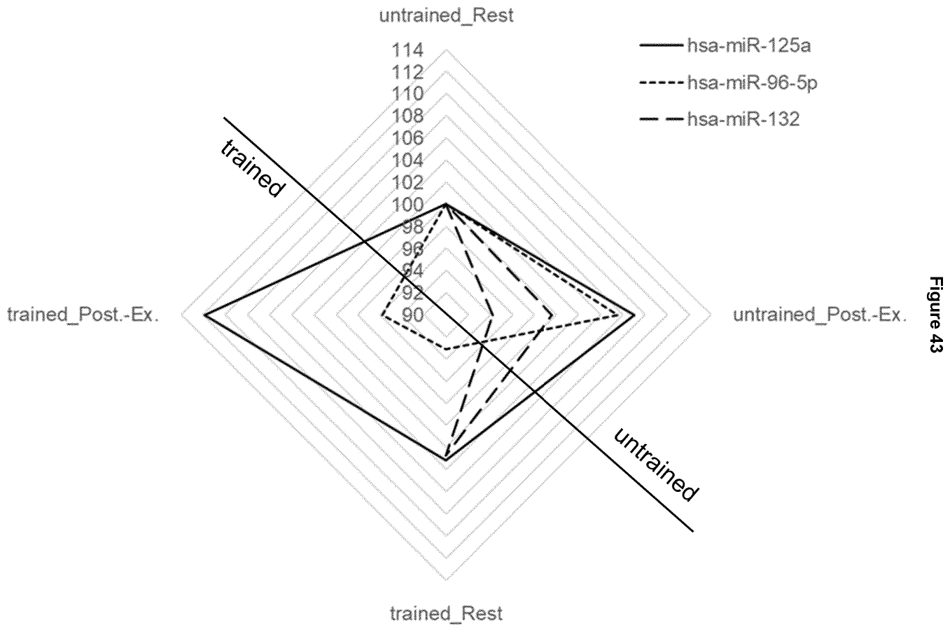

FIG. 44 shows a radar chart (Kiviat diagram) for characterization of acute and sustained exercise training response by combined analysis of miRNA-1, miRNA-132 and miRNA-125a. Combined analysis of miRNA-1, miRNA-132 and miRNA-125a and their respective changes is indicative for whether a training response is present. The difference between conditions is indicated by non-identical shapes on the upper right (untrained) and lower left (trained) side of the chart. “Rest” means that blood was sampled before the start of an acute exercise session, while “Post.-Ex.” means that blood was sampled immediately after an acute exercise session. “Untrained” means that blood was sampled before regular exercise training. “Trained” means that blood was sampled after 4 weeks of regular exercise training. miRNA levels of more than 35 individuals were analyzed for each time point for chart generation. Data has been normalized to the untrained rest condition for each miRNA and fold changes are given.

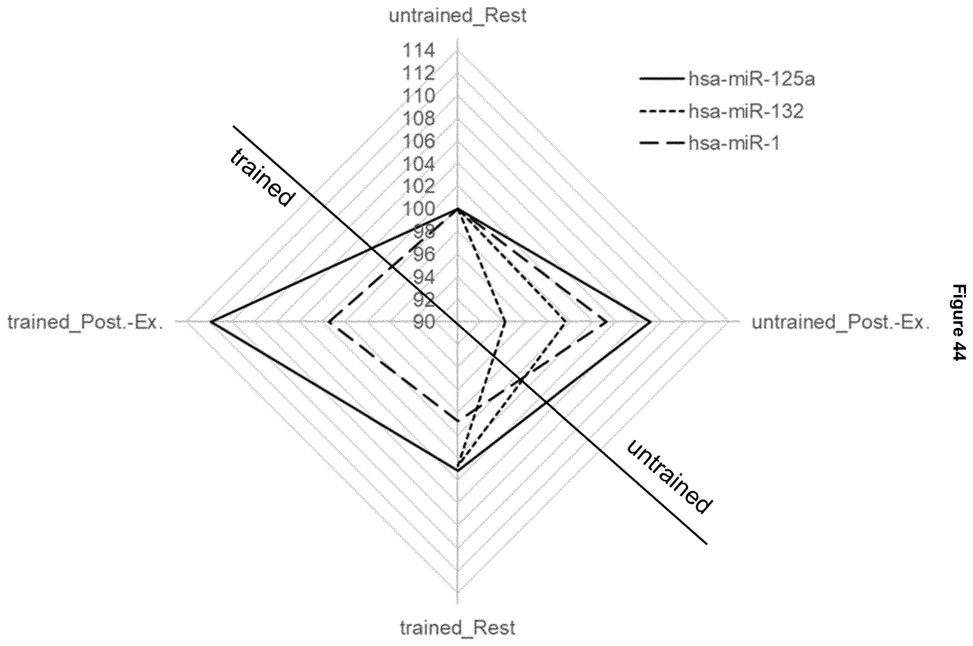

FIG. 45 shows a radar chart (Kiviat diagram) for characterization of acute and sustained exercise training response by combined analysis of miRNA-1, miRNA-96-5p and miRNA-125a. Combined analysis of miRNA-1, miRNA-96-5p and miRNA-125a and their respective changes is indicative for whether a training response is present. The difference between conditions is indicated by non-identical shapes on the upper right (untrained) and lower left (trained) side of the chart. “Rest” means that blood was sampled before the start of an acute exercise session, while “Post.-Ex.” means that blood was sampled immediately after an acute exercise session. “Untrained” means that blood was sampled before regular exercise training. “Trained” means that blood was sampled after 4 weeks of regular exercise training. miRNA levels of more than 35 individuals were analyzed for each time point for chart generation. Data has been normalized to the untrained rest condition for each miRNA and fold changes are given.

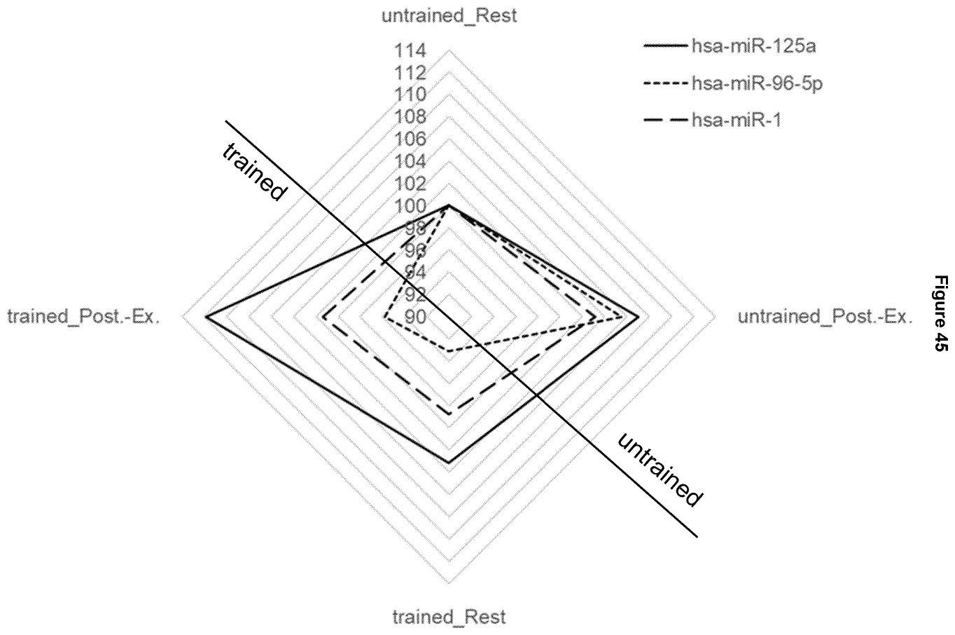

FIG. 46 shows a radar chart (Kiviat diagram) for characterization of acute and sustained exercise training response by combined analysis of miRNA-1, miRNA-98 and miRNA-143. Combined analysis of miRNA-1, miRNA-98 and miRNA-143 and their respective changes is indicative for whether a training response is present. The difference between conditions is indicated by non-identical shapes on the upper right (untrained) and lower left (trained) side of the chart. “Rest” means that blood was sampled before the start of an acute exercise session, while “Post.-Ex.” means that blood was sampled immediately after an acute exercise session. “Untrained” means that blood was sampled before regular exercise training. “Trained” means that blood was sampled after 4 weeks of regular exercise training. miRNA levels of more than 35 individuals were analyzed for each time point for chart generation. Data has been normalized to the untrained rest condition for each miRNA and fold changes are given.

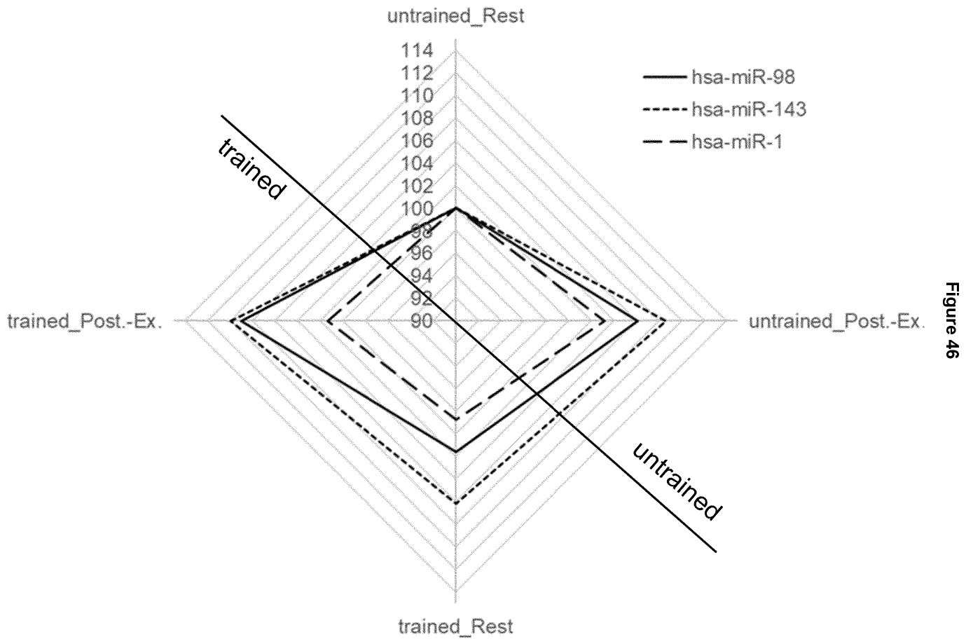

FIG. 47 shows a radar chart (Kiviat diagram) for characterization of acute and sustained exercise training response by combined analysis of miRNA-1, miRNA-24 and miRNA-96-5p. Combined analysis of miRNA-1, miRNA-24 and miRNA-96-5p and their respective changes is indicative for whether a training response is present. The difference between conditions is indicated by non-identical shapes on the upper right (untrained) and lower left (trained) side of the chart. “Rest” means that blood was sampled before the start of an acute exercise session, while “Post.-Ex.” means that blood was sampled immediately after an acute exercise session. “Untrained” means that blood was sampled before regular exercise training. “Trained” means that blood was sampled after 4 weeks of regular exercise training. miRNA levels of more than 35 individuals were analyzed for each time point for chart generation. Data has been normalized to the untrained rest condition for each miRNA and fold changes are given.

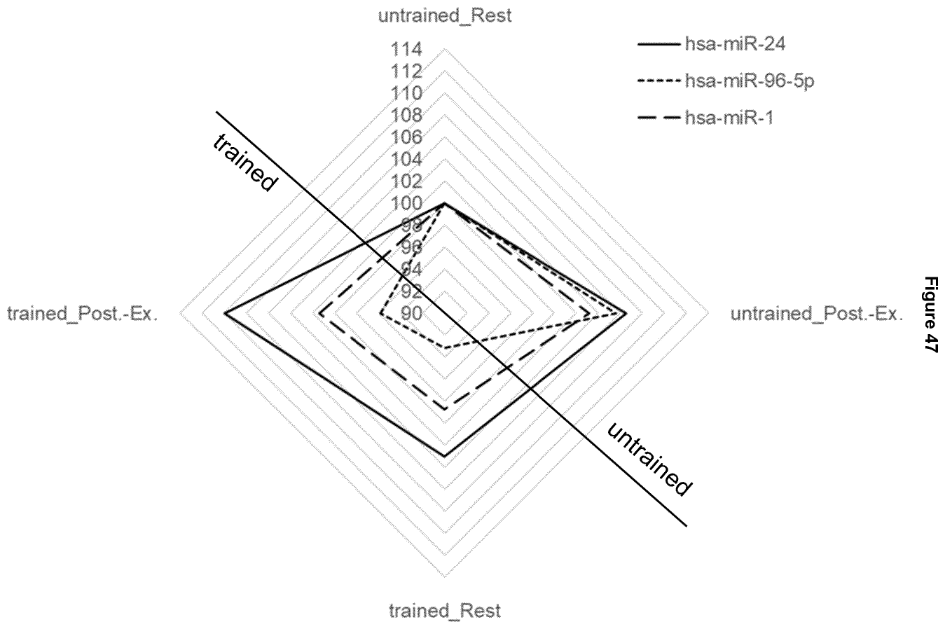

FIG. 48 shows a radar chart (Kiviat diagram) for characterization of acute and sustained exercise training response by combined analysis of miRNA-1, miRNA-125a and miRNA-98p. Combined analysis of miRNA-1, miRNA-125a and miRNA-98 and their respective changes is indicative for whether a training response is present. The difference between conditions is indicated by non-identical shapes on the upper right (untrained) and lower left (trained) side of the chart. “Rest” means that blood was sampled before the start of an acute exercise session, while “Post.-Ex.” means that blood was sampled immediately after an acute exercise session. “Untrained” means that blood was sampled before regular exercise training. “Trained” means that blood was sampled after 4 weeks of regular exercise training. miRNA levels of more than 35 individuals were analyzed for each time point for chart generation. Data has been normalized to the untrained rest condition for each miRNA and fold changes are given.

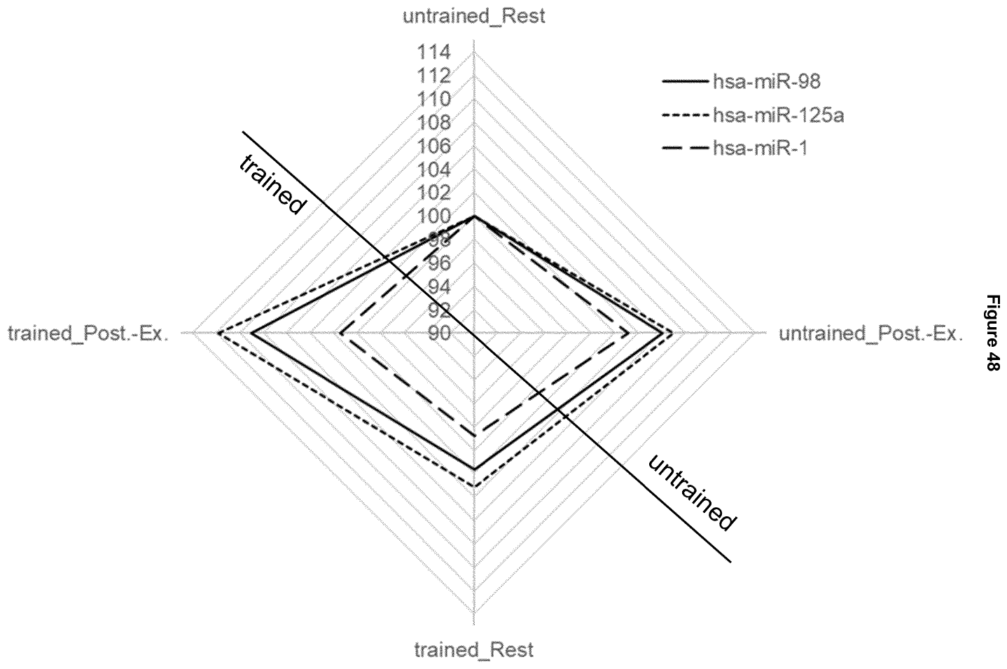

### DETAILED DESCRIPTION OF THE INVENTION

The present invention relates to a method for establishing an individual physical activity program for a subject for reducing an individual risk of the subject for developing a cardiovascular disease, comprising the following steps:
(i) Determining the concentration of at least one circulating miRNA in at least one fluid sample obtained from the subject at least before and after the subject has conducted physical activity, wherein the at least one circulating miRNA is selected from the group consisting of hsa-miRNA-1, hsa-miRNA-24, hsa-miRNA-96, hsa-miRNA-143, hsa-miRNA-98, hsa-miRNA-125a, hsa-miRNA-132, and any combination, sub-combination, portion or fragment thereof;
(ii) comparing the in step (i) determined concentration(s), wherein the result of this comparison is indicative of whether said subject has an individual risk for developing a cardiovascular disease, if the result of this comparison shows an increase or decrease of the concentration of the at least one circulating miRNA after the subject has conducted physical activity compared to the concentration of the at least one circulating miRNA before the subject has conducted physical activity; and
(iii) establishing the individual physical activity program for the subject based on the result of step (ii).

The term “establishing”, as used within the present invention, means to install, to institute, to build or to set up something.

When used within the context of the present invention, the term “physical activity” means any bodily movement produced by skeletal muscles that requires energy expenditure. The energy expenditure can be measured in kilocalories. “Exercise” or “physical exercise”, is a subcategory of physical activity that is planned, structured, repetitive, and purposeful in the sense that the improvement or maintenance of one or more components of physical fitness is the objective. Thus, physical activity includes exercise as well as other activities, which involve bodily movement and are done as part of playing, working, active transportation or recreational activities. In the context of the present invention, it may be that the application of physical activity improves the fitness of a subject, especially, it may be that the physical activity decreases or increases the expression level or the blood concentration level of a circulating miRNA. In the context of the present invention, the physical activity contributes to a decrease or reduction of a risk to develop a cardiovascular disease, wherein the risk may be individual.

In contrast to physical activity, which is related to the movements that people perform, the term “physical fitness”, as used within the present invention, is a set of attributes that people have or achieve. Being “physically fit” can be defined as the ability to carry out daily tasks without undue fatigue. Physical fitness involves different health-related components including cardiorespiratory endurance, muscular endurance, muscular strength, body composition and flexibility.

The term “physical activity program”, as used within the context of the present invention, means a regimen or plan for applying “physical activity” as defined above to be performed by a subject, especially a human subject. A physical activity program in humans is performed to maintain or improve overall physical fitness or to maintain or improve the above mentioned different health-related components of physical fitness individually or in different combinations.

Further, as used within the context of the present invention, the term “establishing a physical activity program” means to install, to institute, to build or to set up a physical activity program as defined above for a subject, most preferably a human subject. Within the present invention, the physical activity program is installed, instituted, built or set up based on the results of a previously conducted blood test for determining the level of one or more circulating miRNA, while the respective physical activity program is then applied to the subject. Further, the term “establishing a physical activity program” also comprises the optional regular physical training according to the created physical activity program.

The term “risk for developing a cardiovascular disease” as used herein refers to the probability, the estimation or the assessment that the subject has or may have for developing a cardiovascular disease. The risk is determined under consideration of the results received from the comparing step (ii). Therefore, the concentration(s) of the respective miRNAs received from step (i) are used. The result of this comparison is indicative whether said subject has a probability of reducing his/her individual risk for developing a cardiovascular disease.

The individual risk to develop a cardiovascular disease may be defined or assessed as being decreased, if the concentration of the hsa-miRNA-1 after the subject has conducted physical activity is increased by at least 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, 80%, 90%, 100%, 200%, 300%, 400%, 500%, 600%, 700%, 800%, 900%, 1000%, 2000%, 3000%, 4000%, 5000%, 6000%, 7000%, 8000%, 9000%, or even 10000%, compared to the concentration of the at least one circulating miRNA before the subject has conducted physical activity.

The individual risk to develop a cardiovascular disease may be defined or assessed as being decreased, if the concentration of hsa-miRNA-24 after the subject has conducted physical activity is increased by at least 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, 80%, 90%, 100%, 200%, 300%, 400%, 500%, 600%, 700%, 800%, 900%, 1000%, 2000%, 3000%, 4000%, 5000%, 6000%, 7000%, 8000%, 9000%, or even 10000%, compared to the concentration of the at least one circulating miRNA before the subject has conducted physical activity.

The individual risk to develop a cardiovascular disease may be defined or assessed as being decreased, if the concentration of hsa-miRNA-96 after the subject has conducted physical activity is increased by at least 1%, 2%, 3%, 4%, 5%, 6%, 7%, 8%, 9%, 10%, 11 12%, 13%, 14%, 15%, 16%, 17%, 18%, 19%, 20%, 30%, 40%, 50%, 60%, 70%, 80%, 90%, 100%, 200%, 300%, 400%, 500%, 600%, 700%, 800%, 900%, 1000%, 2000%, 3000%, 4000%, 5000%, 6000%, 7000%, 8000%, 9000%, or even 10000%, compared to the concentration of the at least one circulating miRNA before the subject has conducted physical activity.

The individual risk to develop a cardiovascular disease may be defined or assessed as being decreased, if the concentration of hsa-miRNA-143 after the subject has conducted physical activity is increased by at least 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, 80%, 90%, 100%, 200%, 300%, 400%, 500%, 600%, 700%, 800%, 900%, 1000%, 2000%, 3000%, 4000%, 5000%, 6000%, 7000%, 8000%, 9000%, or even 10000%, compared to the concentration of the at least one circulating miRNA before the subject has conducted physical activity.

The individual risk to develop a cardiovascular disease may be defined or assessed as being decreased, if the concentration of hsa-miRNA-98 after the subject has conducted physical activity is increased by at least 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, 80%, 90%, 100%, 200%, 300%, 400%, 500%, 600%, 700%, 800%, 900%, 1000%, 2000%, 3000%, 4000%, 5000%, 6000%, 7000%, 8000%, 9000%, or even 10000%, compared to the concentration of the at least one circulating miRNA before the subject has conducted physical activity.

The individual risk to develop a cardiovascular disease may be defined or assessed as being decreased, if the concentration of hsa-miRNA-125a after the subject has conducted physical activity is increased by at least 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, 80%, 90%, 100%, 200%, 300%, 400%, 500%, 600%, 700%, 800%, 900%, 1000%, 2000%, 3000%, 4000%, 5000%, 6000%, 7000%, 8000%, 9000%, or even 10000%, compared to the concentration of the at least one circulating miRNA before the subject has conducted physical activity.

The individual risk to develop a cardiovascular disease may be defined or assessed as being decreased, if the concentration of hsa-miRNA-132 after the subject has conducted physical activity is increased by at least 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, 80%, 90%, 100%, 200%, 300%, 400%, 500%, 600%, 700%, 800%, 900%, 1000%, 2000%, 3000%, 4000%, 5000%, 6000%, 7000%, 8000%, 9000%, or even 10000%, compared to the concentration of the at least one circulating miRNA before the subject has conducted physical activity.

When used within the context of the present invention, the term “miRNA” means a short non-coding RNA molecule of about 22 (18-24) nucleotides that functions in RNA silencing and that post-transcriptionally regulates gene expression and plays important roles in various physiological processes as well as onset and progression of various diseases including cardiovascular disease. A hsa-miRNA is of human origin. Cell-free miRNAs have recently been stably detected in blood and blood components (plasma and serum) as well as other body fluids. Those are called, as used within the context of the present invention, “circulating miRNAs” (short “c-miRNA”).

The term “any combination thereof”, as used within the context of the present invention, means to measure the concentrations of 1, 2, 3, 4, 5, 6 or of all 7 hsa-miRNAs mentioned in the list above consisting of hsa-miRNA-1, hsa-miRNA-24, hsa-miRNA-96, hsa-miRNA-143, hsa-miRNA-98, hsa-miRNA-125a and hsa-miRNA-132.

The term “any sub-combination thereof”, as used within the context of the present invention, means to measure the concentrations of 1, 2, 3, 4, 5, 6 or 7 hsa-miRNAs mentioned in the list above consisting of hsa-miRNA-1, hsa-miRNA-24, hsa-miRNA-96, hsa-miRNA-143, hsa-miRNA-98, hsa-miRNA-125a and hsa-miRNA-132.

The term “any portion thereof”, as used within the context of the present invention, means to measure the concentration(s) of a part/parts or a specific portion/portions of 1, 2, 3, 4, 5, 6 or all 7 hsa-miRNAs mentioned in the list above consisting of hsa-miRNA-1, hsa-miRNA-24, hsa-miRNA-96, hsa-miRNA-143, hsa-miRNA-98, hsa-miRNA-125a and hsa-miRNA-132. For example, the portion may be 1%, 2%, 3%, 4%, 5%, 6%, 7%, 8%, 9%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 99.9% of the respective miRNA or hsa-miRNA.

As used herein, a “portion” or “fragment” of a given microRNA (miRNA) may be any portion of a microRNA and may particularly comprise portions of a microRNA or precursor thereof (e.g., pri- or pre-microRNA) comprising or consisting of at least 10, 11, 12, 13, 14, 15, 16, 17, 18, 19, 20, 21, 22, 23 or 24 consecutive nucleotides of the respective microRNA or precursor thereof. In one embodiment, a portion or fragment of a given microRNA is a portion or fragment, which prevails in the cells after (nuclear and/or cytoplasmic) processing of the microRNA (e.g., pri or pre-microRNAs), e.g., the 5p-arm (also referred to as 5p-strand) or 3p-arm (also referred to as 3p-strand) of the respective miRNA. In one embodiment of the present invention, a portion or fragment of a microRNA is the 5p-arm of a microRNA or its precursor. For example, in accordance with the present invention, a portion or fragment of hsa-miRNA-1 may be inter alia hsa-miRNA-1-5p, and/or a portion or fragment of hsa-miRNA-24 may be inter alia miRNA-24-5p.

The term “portion” may also comprise the forward strand (5′-3′) of the miRNA sequence also termed as miRNA-5p and the reverse strand (3′-5′) of the miRNA sequence also termed as miRNA-3p. In one embodiment of the present invention, the respective miRNA comprises or consists of the 5p-strand and/or the 3p-strand. In one embodiment of the present invention, the respective miRNA comprises or consists of the 5p-strand and the 3p-strand. In one embodiment of the present invention, the respective miRNA comprises of the 5p-strand and the 3p-strand. In one embodiment of the present invention, the respective miRNA consists of the 5p-strand and the 3p-strand. Although both sequences, the one of the 3p-strand and the one of the 5p-strand, may have the same precursor RNA (pre-miRNA) sequence and structure, either the miRNA-5p or the miRNA-3p strand may be functional. The functional strand may be termed guide strand, whereas the non-functional strand may be termed passenger strand. To examine the guide strand, the ability of both strands to bind on a DNA/RNA sequence may be determined. However, miRNA-5p and miRNA-3p may also be present and up-regulated at the same time. Since miRNA-5p and miRNA-3p may have the same pre-miRNA, it may be possible to determine the miRNA concentration by determining the concentration of the pre-miRNA or the primary transcript RNA (pri-RNA).

The term “any fragment thereof”, as used within the context of the present invention, means to measure the concentrations of a specific fragment of 1, 2, 3, 4, 5, 6 or all 7 hsa-miRNAs mentioned in the list above consisting of hsa-miRNA-1, hsa-miRNA-24, hsa-miRNA-96, hsa-miRNA-143, hsa-miRNA-98, hsa-miRNA-125a and hsa-miRNA-132. For example, the fragment may be 1%, 2%, 3%, 4%, 5%, 6%, 7%, 8%, 9%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 99.9% of the respective miRNA or hsa-miRNA. Most preferably, the term “fragment”, means, for example, a specific number of nucleotides resembling a part of the complete respective miRNA or hsa-miRNA sequence.

The term “fluid”, as used within the context of the present invention, refers to any fluid produced by a subject. For example, a fluid may be blood or components thereof, including (but not limited to) sweat, saliva, tear, urine or lymph. The fluid may be used to determine the level of miRNA in the subject.

The term “at least before and after” means, in the context of the present invention, that determining the concentration(s) of the respective miRNA(s) takes place with a sample taken before and after the subject has conducted physical activity (while the step of obtaining the respective sample is not part of the method according to the present invention), also including that this step may be carried out with samples taken several times before or after the subject has conducted physical activity. Additionally, this expression does not exclude that (a) sample(s) for this determining step (i) is/are also received e.g. while the subject conducts physical activity or to any other considerable time point.

The present invention also relates in one embodiment to a method for optimizing an individual physical activity program for a subject for reducing an individual risk of the subject for developing a cardiovascular disease, comprising the following steps: (i) Determining the concentration of at least one circulating miRNA in at least one fluid sample obtained from the subject at least before and after the subject has conducted physical activity, wherein the at least one circulating miRNA is selected from the group consisting of hsa-miRNA-1, hsa-miRNA-24, hsa-miRNA-96, hsa-miRNA-143, hsa-miRNA-98, hsa-miRNA-125a, hsa-miRNA-132, and any combination, sub-combination, portion or fragment thereof; (ii) comparing the in step (i) determined concentration(s), wherein the result of this comparison is indicative of whether said subject has an individual risk for developing a cardiovascular disease, if the result of this comparison shows an increase, maintenance or decrease of the concentration of the at least one circulating miRNA after the subject has conducted physical activity compared to the concentration of the at least one circulating miRNA before the subject has conducted physical activity; and (iii) optimizing the individual physical activity program for the subject based on the result of step (ii). The term “optimizing”, as used within the context of the present invention, means to improve or to ameliorate an already existing status. In the context of the present invention, this means that the comparison step (ii) enables to use the results received in step (i) for improvement of an already existing physical activity program. In one further embodiment, the term “optimizing” may mean to change an existing physical activity program with the intention to increase the effect of the activity program on physical fitness and or the prevention of cardiovascular disease.

The present invention also relates in one embodiment to a method for monitoring an individual physical activity program for a subject for reducing an individual risk of the subject for developing a cardiovascular disease, comprising the following steps: (i) Determining the concentration of at least one circulating miRNA in at least one fluid sample obtained from the subject at least before and after the subject has conducted physical activity, wherein the at least one circulating miRNA is selected from the group consisting of hsa-miRNA-1, hsa-miRNA-24, hsa-miRNA-96, hsa-miRNA-143, hsa-miRNA-98, hsa-miRNA-125a, hsa-miRNA-132, and any combination, sub-combination, portion or fragment thereof; (ii) comparing the in step (i) determined concentration(s), wherein the result of this comparison is indicative of whether said subject has an individual risk for developing a cardiovascular disease, if the result of this comparison shows an increase, maintenance or decrease of the concentration of the at least one circulating miRNA after the subject has conducted physical activity compared to the concentration of the at least one circulating miRNA before the subject has conducted physical activity; and (iii) monitoring the individual physical activity program for the subject based on the result of step (ii). The term “monitoring”, as used within this embodiment of the present invention, means to observe, e.g. a state, a condition or one or several parameters over time. This may also mean in the context of the present invention that the concentration measurements of the respective hsa-miRNA(s) is performed not only before and after the subject has conducted physical activity, but also at additional time points, for example, 7 days, 14 day or 21 days before the subject will conduct physical activity and/or 7 days, 14 days or 21 days after the subject has conducted physical activity.

The present invention also relates in one embodiment to a method for establishing an individual physical activity program for a subject for reducing an individual risk of the subject for developing a cardiovascular disease, comprising the following steps:
(i) Determining the concentration of at least one circulating miRNA in at least one fluid sample obtained from the subject before and after the subject has conducted physical activity, wherein the at least one circulating miRNA is selected from the group consisting of hsa-miRNA-1, hsa-miRNA-24, hsa-miRNA-96, hsa-miRNA-143, hsa-miRNA-98, hsa-miRNA-125a and hsa-miRNA-132;
(ii) comparing the in step (i) determined concentration(s), wherein the result of this comparison is indicative of whether said subject has an individual risk for developing a cardiovascular disease, if the result of this comparison shows an increase of the concentration of the at least one circulating miRNA after the subject has conducted physical activity compared to the concentration of the at least one circulating miRNA before the subject has conducted physical activity; and
(iii) establishing the individual physical activity program for the subject based on the result of step (ii).

The present invention also relates in one embodiment to a method for establishing an individual physical activity program for a subject for reducing an individual risk of the subject for developing a cardiovascular disease, comprising the following steps:
(i) Determining the concentration of at least one circulating miRNA in at least one fluid sample obtained from the subject before and after the subject has conducted physical activity, wherein the at least one circulating miRNA is selected from the group consisting of hsa-miRNA-1, hsa-miRNA-24, hsa-miRNA-96, hsa-miRNA-143, hsa-miRNA-98, hsa-miRNA-125a and hsa-miRNA-132;
(ii) comparing the in step (i) determined concentration(s), wherein the result of this comparison is indicative of whether said subject has an individual risk for developing a cardiovascular disease, if the result of this comparison shows an increase or decrease of the concentration of the at least one circulating miRNA after the subject has conducted physical activity compared to the concentration of the at least one circulating miRNA before the subject has conducted physical activity; and
(iii) establishing the individual physical activity program for the subject based on the result of step (ii).

The present invention also relates in one embodiment to a method for establishing an individual physical activity program for a subject for reducing an individual risk of the subject for developing a cardiovascular disease, comprising the following steps: (i) Determining the concentration of at least one circulating miRNA in at least one fluid sample obtained from the subject before and/or after the subject has conducted physical activity, wherein the at least one circulating miRNA is selected from the group consisting of hsa-miRNA-1, hsa-miRNA-24, hsa-miRNA-96, hsa-miRNA-143, hsa-miRNA-98, hsa-miRNA-125a, hsa-miRNA-132, and any combination, sub-combination, portion or fragment thereof; (ii) comparing the in step (i) determined concentration(s), wherein the result of this comparison is indicative of whether said subject has an individual risk for developing a cardiovascular disease, if the result of this comparison shows an increase or decrease of the concentration of the at least one circulating miRNA after the subject has conducted physical activity compared to the concentration of the at least one circulating miRNA before the subject has conducted physical activity; and (iii) establishing the individual physical activity program for the subject based on the result of step (ii).

The present invention also relates in one embodiment to a method for establishing an individual physical activity program for a subject for reducing an individual risk of the subject for developing a cardiovascular disease, comprising the following steps: (i) Determining the concentration of at least one circulating miRNA in at least one fluid sample obtained from the subject before and/or after the subject has conducted physical activity, wherein the at least one circulating miRNA is selected from the group consisting of hsa-miRNA-1, hsa-miRNA-24, hsa-miRNA-96, hsa-miRNA-143, hsa-miRNA-98, hsa-miRNA-125a, hsa-miRNA-132, and any combination, sub-combination, portion or fragment thereof; (ii) comparing the in step (i) determined concentration(s), wherein the result of this comparison is indicative of whether said subject has an individual risk for developing a cardiovascular disease, if the result of this comparison shows an increase or decrease of the concentration of the at least one circulating miRNA after the subject has conducted physical activity compared to the concentration of the at least one circulating miRNA before the subject has conducted physical activity; and (iii) establishing the individual physical activity program for the subject based on the result of step (ii).

The present invention also relates to a method for establishing an individual physical activity program for a subject for reducing the risk for a cardiovascular disease, comprising the following steps: (i) Determining the concentration of at least one circulating miRNA in at least one fluid sample obtained from the subject at least before and after the subject has conducted physical activity, wherein the at least one circulating miRNA is selected from the group consisting of hsa-miRNA-1, hsa-miRNA-24, hsa-miRNA-96, hsa-miRNA-143, hsa-miRNA-98, hsa-miRNA-125a, hsa-miRNA-132, and any combination, sub-combination, portion or fragment thereof; (ii) comparing the in step (i) determined concentration(s), wherein the result of this comparison is indicative of whether said subject has an individual risk for developing a cardiovascular disease, if the result of this comparison shows an increase of the concentration of the at least one circulating miRNA after the subject has conducted physical activity compared to the concentration of the at least one circulating miRNA before the subject has conducted physical activity; and (iii) establishing the individual physical activity program for the subject based on the result of step (ii).

In a preferred embodiment of the method of the present invention, the subject is a healthy subject. The term “healthy”, as used within the present invention, refers to physical conditions that allow the performance of a physical activity or exercise as defined above. The term “subject”, as used within the present invention, means a human or an animal, wherein the animal may be an ape, a dog, a cat, a cow, a pig, a horse, a camel, a dromedary, a mouse, a rat, a rabbit, a sheep or a goat. In the most preferred embodiment of the method according to the present invention, the subject is a human.

In one embodiment of the method of the present invention, step (i) comprises determining the concentration of 2, 3, 4, 5, 6, or all 7 of the miRNAs selected from the group consisting of hsa-miRNA-1, hsa-miRNA-24, hsa-miRNA-96, hsa-miRNA-143, hsa-miRNA-98, hsa-miRNA-125a, hsa-miRNA-132, and any combination, sub-combination, portion or fragment thereof. In one embodiment of the method of the present invention, step (i) comprises determining the concentration of 2, 3, 4, 5, 6, or all 7 of the miRNAs selected from the group consisting of hsa-miRNA-1, hsa-miRNA-24, hsa-miRNA-96, hsa-miRNA-143, hsa-miRNA-98, hsa-miRNA-125a and hsa-miRNA-132.

In one embodiment of the method of the present invention, step (i) comprises determining the concentration of any of the miRNAs selected from the group consisting of hsa-miRNA-1, hsa-miRNA-143, hsa-miRNA-24, hsa-miRNA-96, and any combination, sub-combination, portion or fragment thereof. In one further preferred embodiment of the method of the present invention, step (i) comprises determining the concentration of any of the miRNAs selected from the group consisting of hsa-miRNA-1, hsa-miRNA-143, hsa-miRNA-24 and hsa-mi RNA-96.

In a preferred embodiment of the method of the present invention, step (i) comprises determining the concentration of any of the miRNAs selected from the group consisting of hsa-miRNA-24, hsa-miRNA-96, and any combination, sub-combination, portion or fragment thereof. In a further preferred embodiment of the method of the present invention, step (i) comprises determining the concentration of any of the miRNAs hsa-miRNA-24 and hsa-mi RNA-96.

In a further preferred embodiment of the method of the present invention, step (i) comprises determining the concentration of any of the miRNAs hsa-miRNA-98 and hsa-miRNA-125a. In a further preferred embodiment of the method of the present invention, step (i) comprises determining the concentration of any of the miRNAs hsa-miRNA-24 and hsa-miRNA-143. In a further preferred embodiment of the method of the present invention, step (i) comprises determining the concentration of any of the miRNAs hsa-miRNA-24, hsa-miRNA-143 and hsa-miRNA-96. In a further preferred embodiment of the method of the present invention, step (i) comprises determining the concentration of any of the miRNAs hsa-miRNA-24, hsa-miRNA-143 and hsa-miRNA-132. In a further preferred embodiment of the method of the present invention, step (i) comprises determining the concentration of any of the miRNAs hsa-miRNA-24, hsa-miRNA-96 and hsa-miRNA-132. In a further preferred embodiment of the method of the present invention, step (i) comprises determining the concentration of any of the miRNAs hsa-miRNA-143, hsa-miRNA-96 and hsa-miRNA-132. In a further preferred embodiment of the method of the present invention, step (i) comprises determining the concentration of any of the miRNAs hsa-miRNA-98 and hsa-miRNA-24. In a further preferred embodiment of the method of the present invention, step (i) comprises determining the concentration of any of the miRNAs hsa-miRNA-98 and hsa-miRNA-143. In a further preferred embodiment of the method of the present invention, step (i) comprises determining the concentration of any of the miRNAs hsa-miRNA-143, hsa-miRNA-98 and hsa-miRNA-132. In a further preferred embodiment of the method of the present invention, step (i) comprises determining the concentration of any of the miRNAs hsa-miRNA-24, hsa-miRNA-98 and hsa-miRNA-132. In a further preferred embodiment of the method of the present invention, step (i) comprises determining the concentration of any of the miRNAs hsa-miRNA-24 and hsa-miRNA-125a. In a further preferred embodiment of the method of the present invention, step (i) comprises determining the concentration of any of the miRNAs hsa-miRNA-143, hsa-miRNA-125a and hsa-miRNA-132. In a further preferred embodiment of the method of the present invention, step (i) comprises determining the concentration of any of the miRNAs hsa-miRNA-24, hsa-miRNA-125a and hsa-miRNA-132. In a further preferred embodiment of the method of the present invention, step (i) comprises determining the concentration of any of the miRNAs hsa-miRNA-96 and hsa-miRNA-98. In a further preferred embodiment of the method of the present invention, step (i) comprises determining the concentration of any of the miRNAs hsa-miRNA-96, hsa-miRNA-125a and hsa-miRNA-98. In a further preferred embodiment of the method of the present invention, step (i) comprises determining the concentration of any of the miRNAs hsa-miRNA-96, hsa-miRNA-24 and hsa-miRNA-98. In a further preferred embodiment of the method of the present invention, step (i) comprises determining the concentration of any of the miRNAs hsa-miRNA-96, hsa-miRNA-143 and hsa-miRNA-98. In a further preferred embodiment of the method of the present invention, step (i) comprises determining the concentration of any of the miRNAs hsa-miRNA-96 and hsa-miRNA-143. In a further preferred embodiment of the method of the present invention, step (i) comprises determining the concentration of any of the miRNAs hsa-miRNA-96, hsa-miRNA-143 and hsa-miRNA-125a. In a further preferred embodiment of the method of the present invention, step (i) comprises determining the concentration of any of the miRNAs hsa-miRNA-98, hsa-miRNA-143 and hsa-miRNA-125a. In a further preferred embodiment of the method of the present invention, step (i) comprises determining the concentration of any of the miRNAs hsa-miRNA-98, hsa-miRNA-24 and hsa-miRNA-125a. In a further preferred embodiment of the method of the present invention, step (i) comprises determining the concentration of any of the miRNAs hsa-miRNA-96, hsa-miRNA-24 and hsa-miRNA-125a. In a further preferred embodiment of the method of the present invention, step (i) comprises determining the concentration of any of the miRNAs hsa-miRNA-96 and hsa-miRNA-125a. In a further preferred embodiment of the method of the present invention, step (i) comprises determining the concentration of any of the miRNAs hsa-miRNA-98, hsa-miRNA-132 and hsa-miRNA-125a. In a further preferred embodiment of the method of the present invention, step (i) comprises determining the concentration of any of the miRNAs hsa-miRNA-96, hsa-miRNA-132 and hsa-miRNA-125a. In a further preferred embodiment of the method of the present invention, step (i) comprises determining the concentration of any of the miRNAs hsa-miRNA-1, hsa-miRNA-132 and hsa-miRNA-125a. In a further preferred embodiment of the method of the present invention, step (i) comprises determining the concentration of any of the miRNAs hsa-miRNA-1, hsa-miRNA-96 and hsa-miRNA-125a. In a further preferred embodiment of the method of the present invention, step (i) comprises determining the concentration of any of the miRNAs hsa-miRNA-1, hsa-miRNA-98 and hsa-miRNA-143. In a further preferred embodiment of the method of the present invention, step (i) comprises determining the concentration of any of the miRNAs hsa-miRNA-1, hsa-miRNA-24 and hsa-miRNA-96. In a further preferred embodiment of the method of the present invention, step (i) comprises determining the concentration of any of the miRNAs hsa-miRNA-1, hsa-miRNA-125a and hsa-miRNA-98.

In one embodiment of the method of the present invention, the at least one circulating miRNA is hsa-miRNA-1 or any portion or fragment thereof. In one preferred embodiment of the method of the present invention, the at least one circulating miRNA is hsa-miRNA-1. In one preferred embodiment of the method of the present invention, hsa-miRNA-1 comprises or consists of the nucleotide sequence according to SEQ ID NO: 1 and/or SEQ ID NO: 2. In one preferred embodiment of the method of the present invention, hsa-miRNA-1 comprises or consists of the nucleotide sequence according to SEQ ID NO: 1. In one preferred embodiment of the method of the present invention, hsa-miRNA-1 comprises or consists of the nucleotide sequence according to SEQ ID NO: 2. In one preferred embodiment of the method of the present invention, hsa-miRNA-1 consists of the nucleotide sequence according to SEQ ID NO: 1 and SEQ ID NO: 2. The sequence of hsa-miRNA-1-3p is given in SEQ ID NO: 1. The sequence of hsa-miRNA-1-5p is given in SEQ ID NO: 2.

In a preferred embodiment of the method of the present invention, the at least one circulating miRNA is hsa-miRNA-24 or any portion or fragment thereof. In one preferred embodiment of the method of the present invention, the at least one circulating miRNA is hsa-miRNA-24. In one preferred embodiment of the method of the present invention, hsa-miRNA-24 comprises or consists of the nucleotide sequence according to SEQ ID NO: 3 and/or SEQ ID NO: 4. In one preferred embodiment of the method of the present invention, hsa-miRNA-24 comprises or consists of the nucleotide sequence according to SEQ ID NO: 3. In one preferred embodiment of the method of the present invention, hsa-miRNA-24 comprises or consists of the nucleotide sequence according to SEQ ID NO: 4. In one preferred embodiment of the method of the present invention, hsa-miRNA-24 consists of the nucleotide sequence according to SEQ ID NO: 3 and SEQ ID NO: 4. The sequence of hsa-miRNA-24-3p is given in SEQ ID NO: 3. The sequence of hsa-miRNA-24-5p is given in SEQ ID NO: 4.

In one preferred embodiment of the method of the present invention, the at least one circulating miRNA is hsa-miRNA-96 or any portion or fragment thereof. In one preferred embodiment of the method of the present invention, the at least one circulating miRNA is hsa-miRNA-96. In one preferred embodiment of the method of the present invention, hsa-miRNA-96 comprises or consists of the nucleotide sequence according to SEQ ID NO: 5 and/or SEQ ID NO: 6. In one preferred embodiment of the method of the present invention, hsa-miRNA-96 comprises or consists of the nucleotide sequence according to SEQ ID NO: 5. In one preferred embodiment of the method of the present invention, hsa-miRNA-96 comprises or consists of the nucleotide sequence according to SEQ ID NO: 6. In one preferred embodiment of the method of the present invention, hsa-miRNA-96 consists of the nucleotide sequence according to SEQ ID NO: 5 and SEQ ID NO: 6. The sequence of hsa-miRNA-96-3p is given in SEQ ID NO: 5. The sequence of hsa-miRNA-96-5p is given in SEQ ID NO: 6.

In a further preferred embodiment of the method of the present invention, the at least one circulating miRNA is hsa-miRNA-143 or any portion or fragment thereof. In one preferred embodiment of the method of the present invention, the at least one circulating miRNA is hsa-miRNA-143. In one preferred embodiment of the method of the present invention, hsa-miRNA-143 comprises or consists of the nucleotide sequence according to SEQ ID NO: 7 and/or SEQ ID NO: 8. In one preferred embodiment of the method of the present invention, hsa-miRNA-143 comprises or consists of the nucleotide sequence according to SEQ ID NO: 7. In one preferred embodiment of the method of the present invention, hsa-miRNA-143 comprises or consists of the nucleotide sequence according to SEQ ID NO: 8. In one preferred embodiment of the method of the present invention, hsa-miRNA-143 consists of the nucleotide sequence according to SEQ ID NO: 7 and SEQ ID NO: 8. The sequence of hsa-miRNA-143-3p is given in SEQ ID NO:7. The sequence of hsa-miRNA-143-5p is given in SEQ ID NO: 8.

In a preferred embodiment of the method of the present invention, the at least one circulating miRNA is hsa-miRNA-98 or any portion or fragment thereof. In one preferred embodiment of the method of the present invention, the at least one circulating miRNA is hsa-miRNA-98. In one preferred embodiment of the method of the present invention, hsa-miRNA-98 comprises or consists of the nucleotide sequence according to SEQ ID NO: 9 and/or SEQ ID NO: 10. In one preferred embodiment of the method of the present invention, hsa-miRNA-98 comprises or consists of the nucleotide sequence according to SEQ ID NO: 9. In one preferred embodiment of the method of the present invention, hsa-miRNA-98 comprises or consists of the nucleotide sequence according to SEQ ID NO: 10. In one preferred embodiment of the method of the present invention, hsa-miRNA-98 consists of the nucleotide sequence according to SEQ ID NO: 9 and SEQ ID NO: 10. The sequence of hsa-miRNA-98-3p is given in SEQ ID NO: 9. The sequence of hsa-miRNA-98-5p is given in SEQ ID NO: 10.

In a further preferred embodiment of the method of the present invention, the at least one circulating miRNA is hsa-miRNA-125a or any portion or fragment thereof. In one preferred embodiment of the method of the present invention, the at least one circulating miRNA is hsa-miRNA-125a. In one preferred embodiment of the method of the present invention, hsa-miRNA-125a comprises or consists of the nucleotide sequence according to SEQ ID NO: 11 and/or SEQ ID NO: 12. In one preferred embodiment of the method of the present invention, hsa-miRNA-125a comprises or consists of the nucleotide sequence according to SEQ ID NO: 11. In one preferred embodiment of the method of the present invention, hsa-miRNA-125a comprises or consists of the nucleotide sequence according to SEQ ID NO: 12. In one preferred embodiment of the method of the present invention, hsa-miRNA-125a consists of the nucleotide sequence according to SEQ ID NO: 11 and SEQ ID NO: 12. The sequence of hsa-miRNA-125a-3p is given in SEQ ID NO: 11. The sequence of hsa-miRNA-125a-5p is given in SEQ ID NO: 12.

In a preferred embodiment of the method of the present invention, the at least one circulating miRNA is hsa-miRNA132 or any portion or fragment thereof. In one preferred embodiment of the method of the present invention, the at least one circulating miRNA is hsa-miRNA-132. In one preferred embodiment of the method of the present invention, hsa-miRNA-132 comprises or consists of the nucleotide sequence according to SEQ ID NO: 13 and/or SEQ ID NO: 14. In one preferred embodiment of the method of the present invention, hsa-miRNA-132 comprises or consists of the nucleotide sequence according to SEQ ID NO: 13. In one preferred embodiment of the method of the present invention, hsa-miRNA-132 comprises or consists of the nucleotide sequence according to SEQ ID NO: 14. In one preferred embodiment of the method of the present invention, hsa-miRNA-132 consists of the nucleotide sequence according to SEQ ID NO: 13 and SEQ ID NO: 14. The sequence of hsa-miRNA-132-3p is given in SEQ ID NO: 13. The sequence of hsa-miRNA-132-5p is given in SEQ ID NO: 14.

In one further embodiment of the method of the present invention, the cardiovascular disease is selected from the group consisting of arteriosclerosis; atherosclerosis; ischemia; endothelial dysfunctions; in particular those dysfunctions affecting blood vessel elasticity; hypertension; peripheral vascular disease; thrombosis; coronary heart disease; heart arrhythmia; heart failure; cardiomyopathy; myocardial infarction; cerebral infarction, renal infarction and restenosis. In another embodiment, the cardiovascular disease may also include those diseases involving chronic inflammatory processes of the vessel wall or elevated pulse wave velocity. In a preferred embodiment of the method of the present invention, the cardiovascular disease is selected from the group consisting of arteriosclerosis; atherosclerosis; ischemia; endothelial dysfunctions; coronary heart disease; myocardial infarction; cerebral infarction, renal infarction and restenosis. In a more preferred embodiment of the method of the present invention, the cardiovascular disease is selected from the group consisting of arteriosclerosis; atherosclerosis; ischemia; coronary heart disease; myocardial infarction; cerebral infarction and renal infarction. In the most preferred embodiment of the method of the present invention, the cardiovascular disease is selected from the group consisting of atherosclerosis, coronary heart disease; myocardial infarction and cerebral infarction.

In an embodiment of the method of the present invention, the concentration of the at least one circulating miRNA is determined with a polymerase chain reaction (PCR)-based screening, such as a real-time quantitative PCR (RT-qPCR), or an immunoassay technique, such as a Northern Blot analysis. Preferably, the at least one circulating miRNA is determined by use of a monoclonal antibody for the detection of DNA/RNA dimers.

In a further embodiment of the method of the present invention, the at least one fluid sample is further obtained from the subject, while the subject conducts physical activity.

In one embodiment of the method of the present invention, the sample is obtained from the subject before the subject has conducted physical activity. For example, the sample is obtained at least 4 weeks before the subject conducts physical activity.

In a further embodiment of the method of the present invention, the sample is obtained from the subject before the subject has conducted physical activity. For example, the sample is obtained at least 24 hours before the subject conducts physical activity.

In one further embodiment of the method of the present invention, the concentration of the at least one circulating miRNA is determined in a regular time schedule, preferably wherein the regular time schedule comprises 2 days to 52 weeks or one week to 10 years.

In a further embodiment of the method of the present invention, the at least one fluid sample is a blood sample, a sample of blood components, a salivary sample, a urine sample, a sweat sample, a tear sample or a lymph sample.

In one embodiment of the method of the present invention, establishing the individual physical activity program for the subject for reducing the individual risk of the subject for developing a cardiovascular disease comprises that the subject receives an assessment about his or her physical fitness as defined above, preferably wherein the assessment is given to the subject by a percent value or by defining a status of fitness as being unchanged, decreased or increased.

In a further embodiment of the method of the present invention, the individual physical activity program is established as high-intensity interval training (HIIT). The term “high-intensity interval training”, as used within the context of the present invention, means a physical activity/exercise at high intensity comprising a period or several subsequent periods of time of a challenging physical activity followed by a period or several subsequent periods of a not challenging physical activity.

In one embodiment of the method of the present invention, the individual physical activity program is established as moderate-intensity training. The term “moderate-intensity training”, as used within the context of the present invention, means a physical activity/exercise at moderate intensity comprising a period of time of a moderate physical activity.

In a further embodiment of the method of the present invention, the individual physical activity program is established as low-intensity training. The term “low-intensity training”, as used within the context of the present invention, means a physical activity/exercise at low intensity comprising a period of time of a not challenging physical activity.

In one embodiment of the method of the present invention, the individual physical activity program is established as isometric training. The term “isometric training”, as used within the context of the present invention, means a physical activity or exercise as defined above, wherein muscle strength is challenged by isometric training applying constant muscle tension.

In a further embodiment of the method of the present invention, the individual physical activity program is established by altering the duration, intensity, number of repetitions or number of sessions of the physical activity. The term “altering”, as used within the present invention, means to adjust, to change or to adapt, especially in the context of the present invention, to adjust, to change or to adapt the physical exercise program under consideration of the determined individual miRNA concentration(s) of a subject. The term “duration”, as used within the present invention, means a time period of 1 s to about 120 min. If HIIT is performed, the training duration may be, for example, about 3 s to about 10 min. In a further example, the HIIT duration may be about 3 s to about 60 min. If a moderate-intensity training or a low-intensity training is performed, the training duration may be, for example, in a range of about 10 min to about 120 min. In one example, the duration time of a moderate-intensity or low-intensity training may be of about 30 min to about 60 min. The training duration of a moderate-intensity or low-intensity training may also be in a range of about 10 min to about 30 min. The term “intensity”, as used within the present invention, refers to the perceived exertion and may be determined by using the Borg rating of perceived exertion scale in the present invention. This numerical scale categorizes the level of exertion during physical activity, wherein the subject performing a training describes the level of perceived exertion. The Borg scale comprises numbers from 6 to 20, wherein number 6 refers to “no exertion at all”, number seven refers to “extremely light”, number 9 refers to “very light”, number 11 refers to “light”, number 13 refers to “somewhat hard”, number 15 refers to “hard”, number 17 refers to “very hard”, number 19 refers to “extremely hard” and number 20 refers to “maximal exertion”. The intermediate numbers are used to express tendencies. In the present invention, the low-intensity training may be rated about 8 to 10. The moderate-intensity training of the present invention may be rated about 10 to 14. The high-intensity training may be rated about 15 to 20. The term “number of repetitions”, as used within the present invention, refers to the repetition of exercise training bouts. In the present invention, the high-interval training may comprise a number of repetitions. For example, the exercises of the high-interval training may be repeated 2 to 60 times. The term “number of sessions”, as used within the present invention, means the number of exercise training sessions in a certain time period. For example, a subject would perform 3 exercise training sessions within one week of time.

The present invention also relates to the use of at least one circulating miRNA in any of the methods according to the present invention. It is preferred in the use of present invention, that the at least one miRNA is selected from the group consisting of hsa-miRNA-1, hsa-miRNA-24, hsa-miRNA-96, hsa-miRNA-143, hsa-miRNA-98, hsa-miRNA-125a, hsa-miRNA-132, and any combination, sub-combination, portion or fragment thereof. It is more preferred for the use of the present invention, that the at least one miRNA is selected from the group consisting of hsa-miRNA-1, hsa-miRNA-24, hsa-miRNA-96, hsa-miRNA-143, hsa-miRNA-98, hsa-miRNA-125a and hsa-miRNA-132.

In one embodiment of the use of the present invention, the at least one miRNA is selected from the group consisting of hsa-miRNA-1, hsa-miRNA-143, hsa-miRNA-24, hsa-miRNA-96, and any combination, sub-combination, portion or fragment thereof. In one preferred embodiment of the use of the present invention, the at least one miRNA is selected from the group consisting of hsa-miRNA-1, hsa-miRNA-143, hsa-miRNA-24 and hsa-miRNA-96.

In one embodiment of the use of the present invention, the at least one miRNA is selected from the group consisting of hsa-miRNA-24, hsa-miRNA-96, and any combination, sub-combination, portion or fragment thereof. In one preferred embodiment of the use of the present invention, the at least one miRNA is hsa-miRNA-24 and hsa-miRNA-96.

In a further preferred embodiment of the use of the present invention, the at least one miRNA are hsa-miRNA-98 and hsa-miRNA-125a. In a further preferred embodiment of the use of the present invention, the at least one miRNA are hsa-miRNA-24 and hsa-miRNA-143. In a further preferred embodiment of the use of the present invention, the at least one miRNA are hsa-miRNA-24, hsa-miRNA-143 and hsa-miRNA-96. In a further preferred embodiment of the use of the present invention, the at least one miRNA are hsa-miRNA-24, hsa-miRNA-143 and hsa-miRNA-132. In a further preferred embodiment of the use of the present invention, the at least one miRNA are hsa-miRNA-24, hsa-miRNA-96 and hsa-miRNA-132. In a further preferred embodiment of the use of the present invention, the at least one miRNA are hsa-miRNA-143, hsa-miRNA-96 and hsa-miRNA-132. In a further preferred embodiment of the use of the present invention, the at least one miRNA are hsa-miRNA-98 and hsa-miRNA-24. In a further preferred embodiment of the use of the present invention, the at least one miRNA are hsa-miRNA-98 and hsa-miRNA-143. In a further preferred embodiment of the use of the present invention, the at least one miRNA are hsa-miRNA-143, hsa-miRNA-98 and hsa-miRNA-132. In a further preferred embodiment of the use of the present invention, the at least one miRNA are hsa-miRNA-24, hsa-miRNA-98 and hsa-miRNA-132. In a further preferred embodiment of the use of the present invention, the at least one miRNA are hsa-miRNA-24 and hsa-miRNA-125a. In a further preferred embodiment of the use of the present invention, the at least one miRNA are hsa-miRNA-143, hsa-miRNA-125a and hsa-miRNA-132. In a further preferred embodiment of the use of the present invention, the at least one miRNA are hsa-miRNA-24, hsa-miRNA-125a and hsa-miRNA-132. In a further preferred embodiment of the use of the present invention, the at least one miRNA are hsa-miRNA-96 and hsa-miRNA-98. In a further preferred embodiment of the use of the present invention, the at least one miRNA are hsa-miRNA-96, hsa-miRNA-125a and hsa-miRNA-98. In a further preferred embodiment of the use of the present invention, the at least one miRNA are hsa-miRNA-96, hsa-miRNA-24 and hsa-miRNA-98. In a further preferred embodiment of the use of the present invention, the at least one miRNA are hsa-miRNA-96, hsa-miRNA-143 and hsa-miRNA-98. In a further preferred embodiment of the use of the present invention, the at least one miRNA are hsa-miRNA-96 and hsa-miRNA-143. In a further preferred embodiment of the use of the present invention, the at least one miRNA are hsa-miRNA-96, hsa-miRNA-143 and hsa-miRNA-125a. In a further preferred embodiment of the use of the present invention, the at least one miRNA are hsa-miRNA-98, hsa-miRNA-143 and hsa-miRNA-125a. In a further preferred embodiment of the use of the present invention, the at least one miRNA are hsa-miRNA-98, hsa-miRNA-24 and hsa-miRNA-125a. In a further preferred embodiment of the use of the present invention, the at least one miRNA are hsa-miRNA-96, hsa-miRNA-24 and hsa-miRNA-125a. In a further preferred embodiment of the use of the present invention, the at least one miRNA are hsa-miRNA-96 and hsa-miRNA-125a. In a further preferred embodiment of the use of the present invention, the at least one miRNA are hsa-miRNA-98, hsa-miRNA-132 and hsa-miRNA-125a. In a further preferred embodiment of the use of the present invention, the at least one miRNA are hsa-miRNA-96, hsa-miRNA-132 and hsa-miRNA-125a. In a further preferred embodiment of the use of the present invention, the at least one miRNA are hsa-miRNA-1, hsa-miRNA-132 and hsa-miRNA-125a. In a further preferred embodiment of the use of the present invention, the at least one miRNA are hsa-miRNA-1, hsa-miRNA-96 and hsa-miRNA-125a. In a further preferred embodiment of the use of the present invention, the at least one miRNA are hsa-miRNA-1, hsa-miRNA-98 and hsa-miRNA-143. In a further preferred embodiment of the use of the present invention, the at least one miRNA are hsa-miRNA-1, hsa-miRNA-24 and hsa-miRNA-96. In a further preferred embodiment of the use of the present invention, the at least one miRNA are hsa-miRNA-1, hsa-miRNA-125a and hsa-miRNA-98.

In a further embodiment of the use of the present invention, the at least one circulating miRNA is hsa-miRNA-1 or a portion or fragment thereof. In a preferred embodiment of the use of the present invention, the at least one circulating miRNA is hsa-miRNA-1. In one preferred embodiment of the use of the present invention, hsa-miRNA-1 comprises or consists of the nucleotide sequence according to SEQ ID NO: 1 and/or SEQ ID NO: 2. In one preferred embodiment of the use of the present invention, hsa-miRNA-1 comprises or consists of the nucleotide sequence according to SEQ ID NO: 1. In one preferred embodiment of the use of the present invention, hsa-miRNA-1 comprises or consists of the nucleotide sequence according to SEQ ID NO: 2. In one preferred embodiment of the use of the present invention, hsa-miRNA-1 consists of the nucleotide sequence according to SEQ ID NO: 1 and SEQ ID NO: 2.

In one embodiment of the use of the present invention, the at least one circulating miRNA is hsa-miRNA-24 or a portion or fragment thereof. In a preferred embodiment of the use of the present invention, the at least one circulating miRNA is hsa-miRNA-24. In one preferred embodiment of the use of the present invention, hsa-miRNA-24 comprises or consists of the nucleotide sequence according to SEQ ID NO: 3 and/or SEQ ID NO: 4. In one preferred embodiment of the use of the present invention, hsa-miRNA-24 comprises or consists of the nucleotide sequence according to SEQ ID NO: 3. In one preferred embodiment of the use of the present invention, hsa-miRNA-24 comprises or consists of the nucleotide sequence according to SEQ ID NO: 4. In one preferred embodiment of the use of the present invention, hsa-miRNA-24 consists of the nucleotide sequence according to SEQ ID NO: 3 and SEQ ID NO: 4.

In one preferred embodiment of the use of the present invention, the at least one circulating miRNA is hsa-miRNA-96 or a portion or fragment thereof. In a preferred embodiment of the use of the present invention, the at least one circulating miRNA is hsa-miRNA-96. In one preferred embodiment of the use of the present invention, hsa-miRNA-96 comprises or consists of the nucleotide sequence according to SEQ ID NO: 5 and/or SEQ ID NO: 6. In one preferred embodiment of the use of the present invention, hsa-miRNA-96 comprises or consists of the nucleotide sequence according to SEQ ID NO: 5. In one preferred embodiment of the use of the present invention, hsa-miRNA-96 comprises or consists of the nucleotide sequence according to SEQ ID NO: 6. In one preferred embodiment of the use of the present invention, hsa-miRNA-96 consists of the nucleotide sequence according to SEQ ID NO: 5 and SEQ ID NO: 6.

In a further embodiment of the use of the present invention, the at least one circulating miRNA is hsa-miRNA-143 or a portion or fragment thereof. In a preferred embodiment of the use of the present invention, the at least one circulating miRNA is hsa-miRNA-143. In one preferred embodiment of the use of the present invention, hsa-miRNA-143 comprises or consists of the nucleotide sequence according to SEQ ID NO: 7 and/or SEQ ID NO: 8. In one preferred embodiment of the use of the present invention, hsa-miRNA-143 comprises or consists of the nucleotide sequence according to SEQ ID NO: 7. In one preferred embodiment of the use of the present invention, hsa-miRNA-143 comprises or consists of the nucleotide sequence according to SEQ ID NO: 8. In one preferred embodiment of the use of the present invention, hsa-miRNA-143 consists of the nucleotide sequence according to SEQ ID NO: 7 and SEQ ID NO: 8.

In one preferred embodiment of the use of the present invention, the at least one circulating miRNA is hsa-miRNA-98 or a portion or fragment thereof. In a preferred embodiment of the use of the present invention, the at least one circulating miRNA is hsa-miRNA-98. In one preferred embodiment of the use of the present invention, hsa-miRNA-98 comprises or consists of the nucleotide sequence according to SEQ ID NO: 9 and/or SEQ ID NO: 10. In one preferred embodiment of the use of the present invention, hsa-miRNA-98 comprises or consists of the nucleotide sequence according to SEQ ID NO: 9. In one preferred embodiment of the use of the present invention, hsa-miRNA-98 comprises or consists of the nucleotide sequence according to SEQ ID NO: 10. In one preferred embodiment of the use of the present invention, hsa-miRNA-98 consists of the nucleotide sequence according to SEQ ID NO: 9 and SEQ ID NO: 10.

In a further embodiment of the use of the present invention, the at least one circulating miRNA is hsa-miRNA-125a or a portion or fragment thereof. In a preferred embodiment of the use of the present invention, the at least one circulating miRNA is hsa-miRNA-125a. In one preferred embodiment of the use of the present invention, hsa-miRNA-125a comprises or consists of the nucleotide sequence according to SEQ ID NO: 11 and/or SEQ ID NO: 12. In one preferred embodiment of the use of the present invention, hsa-miRNA-125a comprises or consists of the nucleotide sequence according to SEQ ID NO: 11. In one preferred embodiment of the use of the present invention, hsa-miRNA-125a comprises or consists of the nucleotide sequence according to SEQ ID NO: 12. In one preferred embodiment of the use of the present invention, hsa-miRNA-125a consists of the nucleotide sequence according to SEQ ID NO: 11 and SEQ ID NO: 12.

In one preferred embodiment of the use of the present invention, the at least one circulating miRNA is hsa-miRNA-132 or a portion or fragment thereof. In a preferred embodiment of the use of the present invention, the at least one circulating miRNA is hsa-miRNA-132. In one preferred embodiment of the use of the present invention, hsa-miRNA-132 comprises or consists of the nucleotide sequence according to SEQ ID NO: 13 and/or SEQ ID NO: 14. In one preferred embodiment of the use of the present invention, hsa-miRNA-132 comprises or consists of the nucleotide sequence according to SEQ ID NO: 13. In one preferred embodiment of the use of the present invention, hsa-miRNA-132 comprises or consists of the nucleotide sequence according to SEQ ID NO: 14. In one preferred embodiment of the use of the present invention, hsa-miRNA-132 consists of the nucleotide sequence according to SEQ ID NO: 13 and SEQ ID NO: 14.

The following sequences are provided herein:

RNA H. sapiens
miRNA-1-3p
SEQ ID NO: 1
5′-UGGAAUGUAAAGAAGUAUGUAU-3′
RNA H. sapiens
miRNA-1-5p
SEQ ID NO: 2
5′-ACAUACUUCUUUAUAUGCCCAU-3′
RNA H. sapiens
miRNA-24-3p
SEQ ID NO: 3
5′-UGGCUCAGUUCAGCAGGAACAG-3′
RNA H. sapiens
miRNA-24-5p
SEQ ID NO: 4
5′-UGCCUACUGAGCUGAUAUCAGU-3′
RNA H. sapiens
miRNA-96-3p
SEQ ID NO: 5
5′-AAUCAUGUGCAGUGCCAAUAUG-3′
RNA H. sapiens
miRNA-96-5p
SEQ ID NO: 6
5′-UUUGGCACUAGCACAUUUUUGCU-3′
RNA H. sapiens
miRNA-143-3p
SEQ ID NO: 7
5′-UGAGAUGAAGCACUGUAGCUC-3′
RNA H. sapiens
miRNA-143-5p
SEQ ID NO: 8
5′-GGUGCAGUGCUGCAUCUCUGGU-3′
RNA H. sapiens
miRNA-98-3p
SEQ ID NO: 9
5′-CUAUACAACUUACUACUUUCCC-3′
RNA H. sapiens
miRNA-98-5p
SEQ ID NO: 10
5′-UGAGGUAGUAAGUUGUAUUGUU-3′
RNA H. sapiens
miRNA-125a-3p
SEQ ID NO: 11
5′-ACAGGUGAGGUUCUUGGGAGCC-3′
RNA H. sapiens
miRNA-125a-5p
SEQ ID NO: 12
5′-UCCCUGAGACCCUUUAACCUGUGA-3′
RNA H. sapiens
miRNA-132-3p
SEQ ID NO: 13
5′-UAACAGUCUACAGCCAUGGUCG-3′
RNA H. sapiens
miRNA-132-5p
SEQ ID NO: 14
5′-ACCGUGGCUUUCGAUUGUUACU-3′
RNA C. elegans
miR-39-3p
SEQ ID NO: 15
5′-UCACCGGGUGUAAAUCAGCUUG-3′

It is noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. The term “at least one” refers, if not particularly defined differently, to one or more such as two, three, four, five, six, seven, eight, nine, ten or more. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “less than” or in turn “more than” does not include the concrete number.

For example, less than 20 means less than the number indicated. Similarly, “more than” or “greater than” means more than or greater than the indicated number, e.g. more than 80% means more than or greater than the indicated number of 80%.

Throughout this specification and the claims which follow, unless the context requires otherwise, the word “comprise”, and variations such as “comprises” and “comprising”, will be understood to imply the inclusion of a stated integer or step or group of integers or steps, but not the exclusion of any other integer or step or group of integer or step. When used herein the term “comprising” can be substituted with the term “containing” or “including” or sometimes when used herein with the term “having”. When used herein “consisting of” excludes any element, step, or ingredient not specified.

The term “including” means “including but not limited to”. “Including” and “including but not limited to” are used interchangeably.

The term “about” means plus or minus 10%, preferably plus or minus 5%, more preferably plus or minus 2%, most preferably plus or minus 1%.

Throughout the description and claims of this specification, the singular encompasses the plural unless the context otherwise requires. In particular, where the indefinite article is used, the specification is to be understood as contemplating plurality as well as singularity, unless the context requires otherwise.

It should be understood that this invention is not limited to the particular methodology, protocols, material, reagents, and substances, etc., described herein and as such can vary. The terminology used herein is for the purpose of describing particular embodiments only, and is not intended to limit the scope of the present invention, which is defined solely by the claims.

All publications cited throughout the text of this specification (including all patents, patent application, scientific publications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

The content of all documents and patent documents cited herein is incorporated by reference in their entirety.

A better understanding of the present invention and of its advantages will be gained from the following examples, offered for illustrative purposes only. The examples are not intended to limit the scope of the present invention in any way.

The invention is further characterized by the following items:

1. A method for establishing an individual physical activity program for a subject for reducing an individual risk of the subject for developing a cardiovascular disease, comprising the following steps:
(i) determining the concentration of at least one circulating miRNA in at least one fluid sample obtained from the subject at least before and after the subject has conducted physical activity,
   wherein the at least one circulating miRNA is selected from the group consisting of hsa-miRNA-1, hsa-miRNA-24, hsa-miRNA-96, hsa-miRNA-143, hsa-miRNA-98, hsa-miRNA-125a, hsa-miRNA-132, and any combination, sub-combination, portion or fragment thereof; and
(ii) comparing the in step (i) determined concentration(s),
   wherein the result of this comparison is indicative of whether said subject has an individual risk for developing a cardiovascular disease,
   if the result of this comparison shows an increase or decrease of the concentration of the at least one circulating miRNA after the subject has conducted physical activity compared to the concentration of the at least one circulating miRNA before the subject has conducted physical activity; and
(iii) establishing the individual physical activity program for the subject based on the result of step (ii).
2. The method of item 1, wherein step (i) comprises determining the concentration of 2, 3, 4, 5, 6, or all 7 of the miRNAs selected from the group consisting of hsa-miRNA-1, hsa-miRNA-24, hsa-miRNA-96, hsa-miRNA-143, hsa-miRNA-98, hsa-miRNA-125a, hsa-miRNA-132, and any combination, sub-combination, portion or fragment thereof.
3. The method of item 1 or item 2, wherein step (i) comprises determining the concentration of any of the miRNAs selected from the group consisting of hsa-miRNA-1, hsa-miRNA-143, hsa-miRNA-24, hsa-miRNA-96, and any combination, sub-combination, portion or fragment thereof.
4. The method of any one of the previous items, wherein step (i) comprises determining the concentration of any of the miRNAs selected from the group consisting of hsa-miRNA-24, hsa-miRNA-96, and any combination, sub-combination, portion or fragment thereof.
5. The method of any one of the previous items, wherein the at least one circulating miRNA is hsa-miRNA-1 or any portion or fragment thereof.
6. The method of item 5, wherein hsa-miRNA-1 comprises or consists of the nucleotide sequence according to SEQ ID NO: 1 and/or SEQ ID NO: 2.
7. The method of any one of the previous items, wherein the at least one circulating miRNA is hsa-miRNA-24 or any portion or fragment thereof.
8. The method of item 7, wherein hsa-miRNA-24 comprises or consists of the nucleotide sequence according to SEQ ID NO: 3 and/or SEQ ID NO: 4.
9. The method of any one of the previous items, wherein the at least one circulating miRNA is hsa-miRNA-96 or any portion or fragment thereof.
10. The method of item 9, wherein hsa-miRNA-96 comprises or consists of the nucleotide sequence according to SEQ ID NO: 5 and/or SEQ ID NO: 6.
11. The method of any one of the previous items, wherein the at least one circulating miRNA is hsa-miRNA-143 or any portion or fragment thereof.
12. The method of item 11, wherein hsa-miRNA-143 comprises or consists of the nucleotide sequence according to SEQ ID NO: 7 and/or SEQ ID NO: 8.
13. The method of any one of the previous items, wherein the at least one circulating miRNA is hsa-miRNA-98 or any portion or fragment thereof.
14. The method of item 13, wherein hsa-miRNA-98 comprises or consists of the nucleotide sequence according to SEQ ID NO: 9 and/or SEQ ID NO: 10.
15. The method of any one of the previous items, wherein the at least one circulating miRNA is hsa-miRNA-125a or any portion or fragment thereof.
16. The method of item 15, wherein hsa-miRNA-125a comprises or consists of the nucleotide sequence according to SEQ ID NO: 11 and/or SEQ ID NO: 12.
17. The method of any one of the previous items, wherein the at least one circulating miRNA is hsa-miRNA-132 or any portion or fragment thereof.
18. The method of item 17, wherein hsa-miRNA-132 comprises or consists of the nucleotide sequence according to SEQ ID NO: 13 and/or SEQ ID NO: 14.
19. The method of any one of the previous items, wherein the cardiovascular disease is selected from the group consisting of arteriosclerosis; atherosclerosis; ischemia; endothelial dysfunctions; in particular those dysfunctions affecting blood vessel elasticity; hypertension; peripheral vascular disease; thrombosis; coronary heart disease; heart arrhythmia; heart failure; cardiomyopathy; myocardial infarction; cerebral infarction, renal infarction and restenosis.
20. The method of any one of the previous items, wherein the concentration of the at least one circulating miRNA is determined with an immunoassay technique, preferably by use of a monoclonal antibody for the detection of DNA/RNA dimers.
21. The method of any one of the previous items, wherein the at least one fluid sample is further obtained from the subject, while the subject conducts physical activity.
22. The method of any one of the previous items, wherein the sample obtained from the subject before the subject has conducted physical activity is obtained at least 4 weeks before the subject conducts physical activity.
23. The method of any one of the previous items, wherein the sample obtained from the subject before the subject has conducted physical activity is obtained at least 24 hours before the subject conducts physical activity.
24. The method of any one of the previous items, wherein the concentration of the at least one circulating miRNA is determined in a regular time schedule, preferably wherein the regular time schedule comprises 2 days to 52 weeks or one week to 10 years.
25. The method of any one of the previous items, wherein the at least one fluid sample is a blood sample, a sample of blood components, a saliva sample, a tear sample, a urine sample, a sweat sample or a lymph sample.
26. The method of any one of the previous items, wherein establishing the individual physical activity program for the subject for reducing the individual risk of the subject for developing a cardiovascular disease comprises that the subject receives an assessment about his or her fitness, preferably wherein the assessment is given to the subject by a percent value or by defining a status of fitness as being unchanged, decreased or increased.
27. The method of any one of the previous items, wherein the individual physical activity program is established as high-intensity interval training.
28. The method of any one of the previous items, wherein the individual physical activity program is established as moderate-intensity training.
29. The method of any one of the previous items, wherein the individual physical activity program is established as low-intensity training.
30. The method of any one of the previous items, wherein the individual physical activity program is established as isometric training.
31. The method of any one of the previous items, wherein the individual physical activity program is established by altering the duration, intensity, number of repetitions or number of sessions of the physical activity.
32. Use of the at least one circulating miRNA in any of the methods according to items 1 to 31.
33. Use according to item 32, wherein the at least one miRNA is selected from the group consisting of hsa-miRNA-1, hsa-miRNA-24, hsa-miRNA-96, hsa-miRNA-143, hsa-miRNA-98, hsa-miRNA-125a, hsa-miRNA-132, and any combination, sub-combination, portion or fragment thereof.
34. Use according to item 32 or 33, wherein the at least one miRNA is selected from the group consisting of hsa-miRNA-1, hsa-miRNA-143, hsa-miRNA-24, hsa-miRNA-96, and any combination, sub-combination, portion or fragment thereof.
35. Use according to any of items 32 to 34, wherein the at least one miRNA is selected from the group consisting of hsa-miRNA-24, hsa-miRNA-96, and any combination, sub-combination, portion or fragment thereof.
36. Use according to any of items 32 to 34, wherein the at least one circulating miRNA is hsa-miRNA-1 or a portion or fragment thereof.
37. The use of item 36, wherein hsa-miRNA-1 comprises or consists of the nucleotide sequence according to SEQ ID NO: 1 and/or SEQ ID NO: 2.
38. Use according to any of items 32 to 34, wherein the at least one circulating miRNA is hsa-miRNA-24 or a portion or fragment thereof.
39. The use of item 38, wherein hsa-miRNA-24 comprises or consists of the nucleotide sequence according to SEQ ID NO: 3 and/or SEQ ID NO: 4.
40. Use according to any of items 32 to 34, wherein the at least one circulating miRNA is hsa-miRNA-96 or a portion or fragment thereof.
41. The use of item 40, wherein hsa-miRNA-96 comprises or consists of the nucleotide sequence according to SEQ ID NO: 5 and/or SEQ ID NO: 6.
42. Use according to any of items 32 to 34, wherein the at least one circulating miRNA is hsa-miRNA-143 or a portion or fragment thereof.
43. The use of item 42, wherein hsa-miRNA-143 comprises or consists of the nucleotide sequence according to SEQ ID NO: 7 and/or SEQ ID NO: 8.
44. Use according to any of items 32 or 33, wherein the at least one circulating miRNA is hsa-miRNA-98 or a portion or fragment thereof.
45. The use of item 44, wherein hsa-miRNA-98 comprises or consists of the nucleotide sequence according to SEQ ID NO: 9 and/or SEQ ID NO: 10.
46. Use according to any of items 32 or 33, wherein the at least one circulating miRNA is hsa-miRNA125a or a portion or fragment thereof.
47. The use of item 46, wherein hsa-miRNA-125a comprises or consists of the nucleotide sequence according to SEQ ID NO: 11 and/or SEQ ID NO: 12.
48. Use according to any of items 32 or 33, wherein the at least one circulating miRNA is hsa-miRNA-132 or a portion or fragment thereof.
49. The use of item 48, wherein hsa-miRNA-132 comprises or consists of the nucleotide sequence according to SEQ ID NO: 13 and/or SEQ ID NO: 14.

### EXAMPLES OF THE INVENTION

The following examples illustrate the invention, but are not to be construed as limiting the scope of the invention.

Methods

Training Protocols: The following trainings protocols have been used in the present invention.

Low-Intensity Exercise is a training protocol where the individual subject performs physical activity/exercise at low intensity. Low-intensity exercise is considered any exercise that induces low physiological strain on the individual subject. It may be performed as walking/running, cycling, swimming or stretching/yoga exercise. It may also involve isometric exercise. Physiological strain may be measured by cardiopulmonary involvement and can be determined by heart rate. For low-intensity exercise, heart rate is typically at 40-50% of maximal heart rate. Physiological strain can also be estimated using a rating of perceived exertion using different scales, such as the 6-20 Borg Scale. On this scale, low intensity would be rated at about 8 to 10. Low-intensity exercise duration may vary between 10 minutes to 120 minutes per session. Low-intensity exercise sessions may be performed on 1 to 7 days a week and may be combined with high-intensity exercise training.

Moderate-Intensity Exercise is a training protocol where the individual subject performs physical activity/exercise at moderate intensity. Moderate-intensity exercise is considered any exercise that induces moderate physiological strain on the individual subject. It may be performed as walking/running, cycling, swimming, rowing etc. Physiological strain may be measured by cardiopulmonary involvement and can be determined by heart rate. For moderate-intensity exercise, heart rate is typically at 50-75% of maximal heart rate. Physiological strain can also be estimated using a rating of perceived exertion using different scales, such as the 6-20 Borg Scale. On this scale, moderate intensity would be rated at about 10 to 14. Moderate-intensity exercise duration may vary between 10 minutes to 120 minutes per session. Moderate-intensity exercise sessions may be performed on 1 to 7 days a week and may be combined with high-intensity exercise training.

High-Intensity Exercise is a training protocol where the individual subject performs physical activity/exercise at high or highest intensity. High-intensity exercise is considered any exercise that induces high or highest physiological strain on the individual subject. It may be performed as running/sprinting, cycling, swimming, rowing etc. Physiological strain may be measured by cardiopulmonary involvement and can be determined by heart rate. For high-intensity exercise, heart rate is typically above 75% of maximal heart rate. Physiological strain can also be estimated using a rating of perceived exertion using different scales, such as the 6-20 Borg Scale. On this scale, high-intensity would be rated at about 15 to 20. High intensity exercise duration may vary between 3 to 10 seconds to 10 minutes per bout. High-intensity exercise may be performed as high-intensity interval training, where exercise bouts at high (or maximal or supramaximal) intensity are interspersed with passive or active (at low or moderate intensity) recovery periods of variable length. Typically, recovery periods would be equal (1:1) or longer (1:2, 1:3, 1:4, etc.) compared to the work periods. High-intensity interval training with shorter work periods (seconds) is also designated as (repeated) sprint training. Work/rest cycle repeats per training sessions may vary from 2-60 repeats dependent on exercise modality, intensity, work duration and rest duration. High-intensity exercise sessions may be performed on 1 to 4 days a week and may be combined with moderate- or low-intensity exercise training.

Identification of miRNAs:

1.1) Central pathological processes and mechanisms known to be involved in the development of cardiovascular disease (CVD) were looked at. Since most CVDs result from the progressive development of atherosclerosis, processes involved in endothelial dysfunction (as starting point of atherosclerosis), vascular inflammation, atherogenesis, arterial calcification and plaque morphology (stable vs. vulnerable plaque) are considered pivotal. Processes are grouped into known underlying cellular mechanisms and signaling pathways including central regulatory molecules.

1.2) To identify functional miRNAs with vasculo- and/or cardio-protective properties, databases on experimental data were then systematically searched using signaling process and regulatory molecule identifiers in combination with the search term ‘microRNA’ (and variations thereof) to identify reports on miRNAs affecting the levels of selected regulatory molecules (transcript and/or protein level) or the overall signaling process. This was done to distinguish miRNAs that may promote the cardiovascular health benefits of regular exercise from miRNAs that are affected by exercise but are involved in the regulation of other processes. The search process was not limited to reports within the field of CVD but was open to other research areas including (but not limited to) molecular biology, general cell biology, cell physiology, development biology, biomedicine (including oncology, nephrology, musculoskeletal medicine, hematology, immunology). Thus, miRNAs from other research fields with reported targets also involved in the development of CVD can be identified and are considered potentially cardio- and/or vasculoprotective as they are anticipated to repress/inhibit or delay pathophysiological processes leading to or associated with CVD. After identification of potential candidate cardio- and/or vasculoprotective miRNAs from the literature, certain quality criteria on available data were applied for restriction. These involve the number of independent reports, data quality, availability of functional analysis, applied model, etc. After these initial selection steps, available whole transcriptome data from analysis of human blood or blood components/fractions (serum, plasma, blood cell-type fractions [mononuclear cells, leucocytes, neutrophils, monocytes, platelets, erythrocytes, natural killer cells, etc.]) were searched for candidate miRNAs to identify circulating miRNAs detectable in the bloodstream. This was performed based on the knowledge that A) mammalian cells secrete miRNAs into the bloodstream, B) intracellular processes are reflected by the specific set (and amount) of miRNAs secreted and C) circulating miRNAs are used for inter-cell communication as they are taken up by target cells and regulate gene expression.

Determination: Candidate miRNAs are then tested in a population of healthy individuals for their abundance in the bloodstream or blood components/fractions (see above) under normal conditions (that is without acute or prior physical activity [>24 h rest]). After positive identification (initial verification of detectable miRNA levels), determination of miRNA concentration changes during acute physical activity is performed. This is based on the knowledge that certain stimuli are induced by physical activity leading to miRNA secretion and subsequently increased miRNA levels in the bloodstream. These include (but may not be limited to) mechanical forces induced by working [skeletal] muscle, mechanical forces induced by the bloodstream mainly on the vascular endothelium, concentration changes of circulating molecules in the bloodstream such as glucose, lactate, as well as blood pH and partial pressure of gases (O₂, CO₂ and NO), concentration of reactive oxygen species and their respective physiological conditions including hypoxia and acidosis. Besides comparison of resting (before) and post-exercise (after) miRNA levels, determination of miRNA levels during exercise (i.e. every 3 min during a prolonged period of exercise) may be performed as miRNA concentration changes have been shown (by the inventor) to be transient (see above, levels affected by secretion and uptake). Moreover, miRNA levels may be determined with respect to their dependence on increasing intensities of physical activity/exercise. To document sustained effects on resting miRNA levels, samples from before and after certain interventional studies are screened.

Cell culture and shear stress experiments: Human umbilical vein endothelial cells (HUVECs) from 3 donors were collected. Cells were grown to confluence at 37° C. with 5% CO₂ on cross-linked gelatin-coated culture plates in endothelial cell growth medium (ECGM; Promocell, Heidelberg, Germany) containing the supplement mix C-39-210 (Promocell) supplemented with 50 mg/ml streptomycin sulphate and 50 U/ml penicillin G (Invitrogen, Darmstadt, Germany). Cells were used for shear stress experiments performed in the cone-and-plate “BioTech Flow” (BTF)-System at 37° C., 5% CO₂. The BTF-System provides constant laminar homogenous flow through circulation of medium provoked by a cone above the culture plate. The inner 10-mm radius of the culture plate was kept free of cells due to non-defined shear rate in the center of the plate. Medium viscosity was increased using 3% polyvinyl-pyrrolidone (MW 360,000; Sigma-Aldrich, Munich, Germany). Subsequently, cells were exposed to different shear rates for up to 1 h in n=3 independent culture plates simultaneously. Shear rates ranged from 0.5 to 30 dyn/cm² according to the range of shear rate reported in the identified array-based analyzes. For in vitro time series analyzes (4 to 60 min, n=3 for each time point), HUVECs from 3 different donors were treated as stated above and were exposed to 30 dyn/cm².

miRNA extraction and quantification: Blood sampling from participants' earlobes was performed. Immediately at the testing site using a 20 μl K2 EDTA capillary (Sarstedt, Nuernbrecht, Germany) and RNA was extracted using 750 μl peqGOLD TriFast (VWR, Darmstadt, Germany) according to the manufacturer's instruction. The applied method allows the detection of acute changes in c-miRNA levels during and directly after exercise and prevents the bias of hemolysis. Each sample was immediately supplemented with 10 nM Caenorhabditis elegans cel-miR-39-3p (SEQ ID No: 15) spike-in control following manufacturer's instruction (Thermo Fisher Scientific, Darmstadt, Germany) for normalization as reported. RNase-free glycogen (70 μg/sample; VWR) was used as carrier to optimize extraction efficiency. Isolated RNA was resuspended in 20 μl of nuclease-free water. RNA from cultured HUVECs was extracted using 2.0 ml peqGOLD TriFast and processed according to the manufacturer's instruction. From each experimental condition, the culture medium was collected completely and RNA from 20 μl conditioned medium was extracted using 750 μl peqGOLD TriFast as described above and re-suspended in 20 μl RNase-free water. Quantification of mature hsa-miRNAs was performed by quantitative real-time polymerase chain reaction (qRT-PCR) using 5′ adaptor ligation and target-independent cDNA generation in a single reaction (TaqMan Advanced MicroRNA technology; Thermo Fisher Scientific, Darmstadt, Germany). In brief, 1.0 μl of RNA solution was used for adaptor ligation and reverse transcription according to manufacturer's instructions. cDNA was diluted 1:10 in ultra-pure water and 1.25 μl were used for final qRT-PCR reactions performed in a 384-well format in duplicates on an ABI7500 fast RT-PCR system (Life Technologies, Carlsbad, USA). Relative quantification was performed using the ΔCt method and miRNA values were expressed as (1/ΔCt)*100 for presentation. Duplicates with a difference greater than 2 Ct were excluded from the analysis.

miRNA profiles: For evaluation of the training status of an individual in terms of practical application, the following steps were performed: Levels of identified vasculo- and cardio-protective miRNAs were determined in groups of healthy trained and untrained individuals. Training status of the individuals was analyzed using a standard fitness test including blood lactate diagnostics and heart rate analysis for estimation of physical exercise capacity. For each identified miRNA, capillary blood miRNA levels from at least 30 trained and untrained individuals were determined at rest and after performing a standardized exercise test. Thus, information on miRNA levels from four different conditions was available: Untrained individuals at rest, untrained individuals after standardized acute exercise, trained individuals at rest and trained individuals after standardized acute exercise. Levels of each individual miRNA were then normalized to the untrained rest condition and indicated as arbitrary units. Respective miRNA levels for all other conditions were expressed as fold changes compared to the untrained rest condition. From this data, standard radar charts (Kiviat diagrams) were generated, which present the difference between trained and untrained individuals based on miRNA levels by comparing miRNA levels before and after an individual has performed exercise. The difference between conditions is indicated by non-identical geometric shapes on the upper right (untrained) and lower left (trained) side of the chart. Analysis of miRNAs and their combinations allows the distinction between trained and untrained individuals based on the generated reference profiles.

### REFERENCES

1. Flowers, E., Won, G. Y. and Fukuoka, Y. (2015). MicroRNAs associated with exercise and diet: a systematic review. Physiol. Genomics. 47, 1-11.
2. Polakovic\check{o}vá, M., Musil, P., Laczo, E., Hamar, D. and Kyselovic, J. (2016). Circulating MicroRNAs as Potential Biomarkers of Exercise Response. Int. J. Mol. Sci. 306, H557-H563.
3. Silva, G. J. J., Bye, A., El Azzouzi, H. and Wisløff, U. (2017). MicroRNAs as Important Regulators of Exercise Adaptation. Prog. Cardiovasc. Dis. 60, 130-151.
4. Sapp, R. M., Shill, D. D., Roth, S. M. and Hagberg, J. M. (2016). Circulating microRNAs in acute and chronic exercise: more than mere biomarkers. J. Appl. Physiol. (1985) 122, 702-717.
5. Bartel, D. P. (2018). Metazoan MicroRNAs. Cell 173, 20-51.
6. Filipowicz, W., Bhattacharyya, S. N. and Sonenberg, N. (2008). Mechanisms of post transcriptional regulation by microRNAs: are the answers in sight? Nat. Rev. Genet. 9, 102-114.
7. Huntzinger, E. and Izaurralde, E. (2011). Gene silencing by microRNAs: contributions of translational repression and mRNA decay. Nat. Rev. Genet. 12, 99-110.
8. Alvarez-Garcia, I. and Miska, E. A. (2005). MicroRNA functions in animal development and human disease. Development 132, 4653-4662.
9. Jung, H. J. and Suh, Y. (2014). Circulating miRNAs in ageing and ageing-related diseases. J. Genet. Genomics 41, 465-472.
10. Sayed, D. and Abdellatif, M. (2011). MicroRNAs in development and disease. Physiol. Rev. 91, 827-887.
11. Friedman, R. C., Farh, K. K., Burge, C. B., Bartel, D. P. (2009). Most mammalian mRNAs are conserved targets of microRNAs. Genome Res. 19, 92-105.
12. de Gonzalo-Calvo, D., Vea, A., Bär, C., Fiedler, J., Couch, L. S., Brotons, C., Llorente-Cortes, V., Thum, T. (2018). Circulating non-coding RNAs in biomarker-guided cardiovascular therapy: a novel tool for personalized medicine? Eur. Heart J.
13. Arroyo, J. D., Chevillet, J. R., Kroh, E. M., Ruf, I. K., Pritchard, C. C., Gibson, D. F., Bennett, C. F., Pogosova-Agadjanyan, E. L., Stirewalt, D. L., Tait, J. F., Tewari, M. (2011). Argonaute2 complexes carry a population of circulating microRNAs independent of vesicles in human plasma. Proc. Natl. Acad. Sci. U.S.A 108, 5003-5008.
14. Deregibus, M. C., Cantaluppi, V., Calogero, R., Lo Iacono, M., Tetta, C., Biancone, L., Bruno, S., Bussolati, B., Camussi, G. (2007). Endothelial progenitor cell derived microvesicles activate an angiogenic program in endothelial cells by a horizontal transfer of mRNA. Blood 110, 2440-2448.
15. Kumar, S., Kim, C. W., Simmons, R. D., Jo, H. (2014). Role of flow-sensitive microRNAs in endothelial dysfunction and atherosclerosis: mechanosensitive athero-miRs. Arterioscler. Thromb. Vasc. Biol. 34, 2206-2216.
16. Nallamshetty, S., Chan, S. Y., Loscalzo, J. (2013). Hypoxia: a master regulator of microRNA biogenesis and activity. Free Radic. Biol. Med. 64, 20-30.
17. Bandara, K. V., Michael, M. Z., Gleadle, J. M. (2017). MicroRNA Biogenesis in Hypoxia. Microrna 6, 80-96.
18. Newcomer, S. C., Thijssen, D. H. and Green, D. J. (2011). Effects of exercise on endothelium and endothelium/smooth muscle cross talk: role of exercise-induced hemodynamics. J. Appl. Physiol. (1985) 111, 311-320.
19. Thijssen, D. H., Dawson, E. A., Black, M. A., Hopman, M. T., Cable, N. T. and Green, D. J. (2009). Brachial artery blood flow responses to different modalities of lower limb exercise. Med. Sci. Sports Exerc. 41, 1072-1079.
20. Green, D. J., Hopman, M. T., Padilla, J., Laughlin, M. H., Thijssen, D. H. (2017). Vascular Adaptation to Exercise in Humans: Role of Hemodynamic Stimuli. Physiol. Rev. 97, 495-528.
21. Tinken, T. M., Thijssen, D. H., Hopkins, N., Dawson, E. A., Cable, N. T. and Green, D. J. (2010). Shear stress mediates endothelial adaptations to exercise training in humans. Hypertension 55, 312-318.
22. Hambrecht, R., Adams, V., Erbs, S., Linke, A., Krankel, N., Shu, Y., Baither, Y., Gielen, S., Thiele, H., Gummert, J. F., Mohr, F. W., Schuler, G. (2003). Regular physical activity improves endothelial function in patients with coronary artery disease by increasing phosphorylation of endothelial nitric oxide synthase. Circulation 107, 3152-3158.
23. Seebach, J., Donnert, G., Kronstein, R., Werth, S., Wojciak-Stothard, B., Falzarano, D., Mrowietz, C., Hell, S. W., Schnittler, H. J. (2007). Regulation of endothelial barrier function during flow-induced conversion to an arterial phenotype. Cardiovasc. Res. 75, 596-607.
24. Mewes, M., Nedele, J., Schelleckes, K., Bondareva, O., Lenders, M., Kusche-Vihrog, K., Schnittler, H. J., Brand, S. M., Schmitz, B., Brand, E. (2017). Salt-induced Na(+)/K(+)-ATPase-α/β expression involves soluble adenylyl cyclase in endothelial cells. Pflugers Arch. 469, 1401-1412.
25. Kusche-Vihrog, K., Schmitz, B., Brand, E. (2015) Salt controls endothelial and vascular phenotype. Pflugers Arch. 467, 499-512.
26. Abe, J. and Berk, B. C. (2014). Novel mechanisms of endothelial mechanotransduction. Arterioscler. Thromb. Vasc. Biol. 34, 2378-2386.
27. Vozzi, F., Campolo, J., Cozzi, L., Politano, G., Di Carlo, S., Rial, M., Comenici, C., Parodi, O. (2018). Computing of Low Shear Stress-Driven Endothelial Gene Network Involved in Early Stages of Atherosclerotic Process. Biomed Res Int. 2018:5359839.
28. Schober, A., Nazari-Jahantigh, M., Wie, Y., Bidzhekov, K., Gremse, F., Grommes, J., Megens, R. T., Heyll, K., Noels, H., Hristov, M., Wang, S., Kiessling, F., Olson, E. N. et al. (2014). MicroRNA-126-5p promotes endothelial proliferation and limits atherosclerosis by suppressing DIk1. Nat. Med. 20, 368-376.
29. Cordes, K. R., Sheehy, N. T., White, M. P., Berry, E. C., Morton, S. U., Muth, A. N., Lee, T. H., Miano, J. M., Ivey, K. N., Srivastava, D. (2009). miR-145 and miR-143 regulate smooth muscle cell fate and plasticity. Nature 460, 705-710.
30. Chen, X., Liang, H., Zhang, J., Zen, K. and Zhang, C. Y. (2012). Secreted microRNAs: a new form of intercellular communication. Trends Cell. Biol. 22, 125-132.
31. Makarova, J. A., Maltseva, D. V., Galatenko, V. V., Abbasi, A., Maximenko, D. G., Grigoriev, A. I., Tonevitsky, A. G., Northoff, H. (2014). Exercise immunology meets MiRNAs. Exerc. Immunol. Rev. 20, 135-164.
32. Zhang, J., Li, S., Li, L., Li, M., Guo, C., Yao, J., Mi, S. (2015). Exosome and exosomal microRNA: trafficking, sorting, and function. Genomics Proteomics Bioinformatics 13, 17-24.
33. de Jong, 0. G., Verhaar, M. C., Chen, Y., Vader, P., Gremmels, H., Posthuma, G., Schiffelers, R. M., Gucek, M., van Balkom, B. W. (2012). Cellular stress conditions are reflected in the protein and RNA content of endothelial cell-derived exosomes. J. Extracell. Vesicles. 16:18396.
34. Hergenreider, E., Heydt, S., Tréguer, K., Boettger, T., Horrevoets, A. J., Zeiher, A. M., Scheffer, M. P., Frangakis, A. S., Yin, X., Mayr, M., Braun, T., Urbich, C., Boon, R. A., et al. (2012). Atheroprotective communication between endothelial cells and smooth muscle cells through miRNAs. Nat. Cell. Biol. 14, 249-256. 1
35. Milanovic, Z., Sporis, G. and Weston, M. (2015). Effectiveness of High-Intensity Interval Training (HIT) and Continuous Endurance Training for VO2max Improvements: A Systematic Review and Meta-Analysis of Controlled Trials. Sports Med. 45, 1469-1481.
36. Weston, K. S., Wisløff, U. and Coombes, J. S. (2014). High-intensity interval training in patients with lifestyle-induced cardiometabolic disease: a systematic review and meta-analysis. Br. J. Sports Med. 48, 1227-1234.
37. Sloth, M., Sloth, D., Overgaard, K. and Dalgas, U. (2013). Effects of sprint interval training on VO2max and aerobic exercise performance: Asystematic review and meta-analysis. Scand. J. Med. Sci. Sports 23:e341-352.

1. A method for establishing an individual physical activity program for a subject for reducing an individual risk of the subject for developing a cardiovascular disease, comprising the following steps:
(i) determining the concentration of at least one circulating miRNA in at least one fluid sample obtained from the subject at least before and after the subject has conducted physical activity,
wherein the at least one circulating miRNA is selected from the group consisting of hsa-miRNA-1, hsa-miRNA-24, hsa-miRNA-96, hsa-miRNA-143, hsa-miRNA-98, hsa-miRNA-125a, hsa-miRNA-132, and any combination, sub-combination, portion or fragment thereof; and
(ii) comparing the in step (i) determined concentration(s),
wherein the result of this comparison is indicative of whether said subject has an individual risk for developing a cardiovascular disease,
if the result of this comparison shows an increase or decrease of the concentration of the at least one circulating miRNA after the subject has conducted physical activity compared to the concentration of the at least one circulating miRNA before the subject has conducted physical activity; and
(iii) establishing the individual physical activity program for the subject based on the result of step (ii).

2. The method of claim 1, wherein step (i) comprises determining the concentration of 2, 3, 4, 5, 6, or all 7 of the miRNAs selected from the group consisting of hsa-miRNA-1, hsa-miRNA-24, hsa-miRNA-96, hsa-miRNA-143, hsa-miRNA-98, hsa-miRNA-125a, hsa-miRNA-132, and any combination, sub-combination, portion or fragment thereof.

3. The method of claim 1 or claim 2, wherein step (i) comprises determining the concentration of any of the miRNAs selected from the group consisting of hsa-miRNA-1, hsa-miRNA-143, hsa-miRNA-24, hsa-miRNA-96, and any combination, sub-combination, portion or fragment thereof.

4. The method of any one of the previous claims, wherein step (i) comprises determining the concentration of any of the miRNAs selected from the group consisting of hsa-miRNA-24, hsa-miRNA-96, and any combination, sub-combination, portion or fragment thereof.

5. The method of any one of the previous claims, wherein the at least one circulating miRNA is hsa-miRNA-1 or any portion or fragment thereof.

6. The method of claim 5, wherein hsa-miRNA-1 comprises or consists of the nucleotide sequence according to SEQ ID NO: 1 and/or SEQ ID NO: 2.

7. The method of any one of the previous claims, wherein the at least one circulating miRNA is hsa-miRNA-24 or any portion or fragment thereof.

8. The method of claim 7, wherein hsa-miRNA-24 comprises or consists of the nucleotide sequence according to SEQ ID NO: 3 and/or SEQ ID NO: 4.

9. The method of any one of the previous claims, wherein the at least one circulating miRNA is hsa-miRNA-96 or any portion or fragment thereof.

10. The method of claim 9, wherein hsa-miRNA-96 comprises or consists of the nucleotide sequence according to SEQ ID NO: 5 and/or SEQ ID NO: 6.

11. The method of any one of the previous claims, wherein the at least one circulating miRNA is hsa-miRNA-143 or any portion or fragment thereof.

12. The method of claim 11, wherein hsa-miRNA-143 comprises or consists of the nucleotide sequence according to SEQ ID NO: 7 and/or SEQ ID NO: 8.

13. The method of any one of the previous claims, wherein the at least one circulating miRNA is hsa-miRNA-98 or any portion or fragment thereof.

14. The method of claim 13, wherein hsa-miRNA-98 comprises or consists of the nucleotide sequence according to SEQ ID NO: 9 and/or SEQ ID NO: 10.

15. The method of any one of the previous claims, wherein the at least one circulating miRNA is hsa-miRNA-125a or any portion or fragment thereof.

16. The method of claim 15, wherein hsa-miRNA-125a comprises or consists of the nucleotide sequence according to SEQ ID NO: 11 and/or SEQ ID NO: 12.

17. The method of any one of the previous claims, wherein the at least one circulating miRNA is hsa-miRNA-132 or any portion or fragment thereof.

18. The method of claim 17, wherein hsa-miRNA-132 comprises or consists of the nucleotide sequence according to SEQ ID NO: 13 and/or SEQ ID NO: 14.

19. The method of any one of the previous claims, wherein the cardiovascular disease is selected from the group consisting of arteriosclerosis; atherosclerosis; ischemia; endothelial dysfunctions; in particular those dysfunctions affecting blood vessel elasticity; hypertension; peripheral vascular disease; thrombosis; coronary heart disease; heart arrhythmia; heart failure; cardiomyopathy; myocardial infarction; cerebral infarction, renal infarction and restenosis.

20. The method of any one of the previous claims, wherein the concentration of the at least one circulating miRNA is determined with an immunoassay technique, preferably by use of a monoclonal antibody for the detection of DNA/RNA dimers.

21. The method of any one of the previous claims, wherein the at least one fluid sample is further obtained from the subject, while the subject conducts physical activity.

22. The method of any one of the previous claims, wherein the sample obtained from the subject before the subject has conducted physical activity is obtained at least 4 weeks before the subject conducts physical activity.

23. The method of any one of the previous claims, wherein the sample obtained from the subject before the subject has conducted physical activity is obtained at least 24 hours before the subject conducts physical activity.

24. The method of any one of the previous claims, wherein the concentration of the at least one circulating miRNA is determined in a regular time schedule, preferably wherein the regular time schedule comprises 2 days to 52 weeks or one week to 10 years.

25. The method of any one of the previous claims, wherein the at least one fluid sample is a blood sample, a sample of blood components, a saliva sample, a tear sample, a urine sample, a sweat sample or a lymph sample.

26. The method of any one of the previous claims, wherein establishing the individual physical activity program for the subject for reducing the individual risk of the subject for developing a cardiovascular disease comprises that the subject receives an assessment about his or her fitness, preferably wherein the assessment is given to the subject by a percent value or by defining a status of fitness as being unchanged, decreased or increased.

27. The method of any one of the previous claims, wherein the individual physical activity program is established as high-intensity interval training.

28. The method of any one of the previous claims, wherein the individual physical activity program is established as moderate-intensity training.

29. The method of any one of the previous claims, wherein the individual physical activity program is established as low-intensity training.

30. The method of any one of the previous claims, wherein the individual physical activity program is established as isometric training.

31. The method of any one of the previous claims, wherein the individual physical activity program is established by altering the duration, intensity, number of repetitions or number of sessions of the physical activity.

32. Use of the at least one circulating miRNA in any of the methods according to claims 1 to 31.

33. Use according to claim 32, wherein the at least one miRNA is selected from the group consisting of hsa-miRNA-1, hsa-miRNA-24, hsa-miRNA-96, hsa-miRNA-143, hsa-miRNA-98, hsa-miRNA-125a, hsa-miRNA-132, and any combination, sub-combination, portion or fragment thereof.

34. Use according to claim 32 or 33, wherein the at least one miRNA is selected from the group consisting of hsa-miRNA-1, hsa-miRNA-143, hsa-miRNA-24, hsa-miRNA-96, and any combination, sub-combination, portion or fragment thereof.

35. Use according to any of claims 32 to 34, wherein the at least one miRNA is selected from the group consisting of hsa-miRNA-24, hsa-miRNA-96, and any combination, sub-combination, portion or fragment thereof.

36. Use according to any of claims 32 to 34, wherein the at least one circulating miRNA is hsa-miRNA-1 or a portion or fragment thereof.

37. The use of claim 36, wherein hsa-miRNA-1 comprises or consists of the nucleotide sequence according to SEQ ID NO: 1 and/or SEQ ID NO: 2.

38. Use according to any of claims 32 to 34, wherein the at least one circulating miRNA is hsa-miRNA-24 or a portion or fragment thereof.

39. The use of claim 38, wherein hsa-miRNA-24 comprises or consists of the nucleotide sequence according to SEQ ID NO: 3 and/or SEQ ID NO: 4.

40. Use according to any of claims 32 to 34, wherein the at least one circulating miRNA is hsa-miRNA-96 or a portion or fragment thereof.

41. The use of claim 40, wherein hsa-miRNA-96 comprises or consists of the nucleotide sequence according to SEQ ID NO: 5 and/or SEQ ID NO: 6.

42. Use according to any of claims 32 to 34, wherein the at least one circulating miRNA is hsa-miRNA-143 or a portion or fragment thereof.

43. The use of claim 42, wherein hsa-miRNA-143 comprises or consists of the nucleotide sequence according to SEQ ID NO: 7 and/or SEQ ID NO: 8.

44. Use according to any of claim 32 or 33, wherein the at least one circulating miRNA is hsa-miRNA-98 or a portion or fragment thereof.

45. The use of claim 44, wherein hsa-miRNA-98 comprises or consists of the nucleotide sequence according to SEQ ID NO: 9 and/or SEQ ID NO: 10.

46. Use according to any of claim 32 or 33, wherein the at least one circulating miRNA is hsa-miRNA125a or a portion or fragment thereof.

47. The use of claim 46, wherein hsa-miRNA-125a comprises or consists of the nucleotide sequence according to SEQ ID NO: 11 and/or SEQ ID NO: 12.

48. Use according to any of claim 32 or 33, wherein the at least one circulating miRNA is hsa-miRNA-132 or a portion or fragment thereof.

49. The use of claim 48, wherein hsa-miRNA-132 comprises or consists of the nucleotide sequence according to SEQ ID NO: 13 and/or SEQ ID NO: 14.


SEQUENCE LISTING (15 sequences)

SEQ ID NO: 1 - RNA, 22 nt, human
uggaauguaaagaaguauguau

SEQ ID NO: 2 - RNA, 22 nt, human
acauacuucuuuauaugcccau

SEQ ID NO: 3 - RNA, 22 nt, human
uggcucaguucagcaggaacag

SEQ ID NO: 4 - RNA, 22 nt, human
ugccuacugagcugauaucagu

SEQ ID NO: 5 - RNA, 22 nt, human
aaucaugugcagugccaauaug

SEQ ID NO: 6 - RNA, 23 nt, human
uuuggcacuagcacauuuuugcu

SEQ ID NO: 7 - RNA, 21 nt, human
ugagaugaagcacuguagcuc

SEQ ID NO: 8 - RNA, 22 nt, human
ggugcagugcugcaucucuggu

SEQ ID NO: 9 - RNA, 22 nt, human
cuauacaacuuacuacuuuccc

SEQ ID NO: 10 - RNA, 22 nt, human
ugagguaguaaguuguauuguu

SEQ ID NO: 11 - RNA, 22 nt, human
acaggugagguucuugggagcc

SEQ ID NO: 12 - RNA, 24 nt, human
ucccugagacccuuuaaccuguga

SEQ ID NO: 13 - RNA, 22 nt, human
uaacagucuacagccauggucg

SEQ ID NO: 14 - RNA, 22 nt, human
accguggcuuucgauuguuacu

SEQ ID NO: 15 - RNA, 22 nt, Caenorhabditis elegans
ucaccggguguaaaucagcuug